(19) United States
(12) Patent Application Publication
(10) Pub. No.: US 2014/0243374 A1
(43) Pub. Date: Aug. 28, 2014

(54) Novel Sulfonamides

(71) Applicant: The Brigham and Women's Hospital, Inc., Boston, MA (US)

(72) Inventors: Corinne E. Augelli-Szafran, West Newton, MA (US); Dai Lu, Boston, MA (US); HanXun Wei, West Roxbury, MA (US); Jing Zhang, Lexington, MA (US); Michael S. Wolfe, West Newton, MA (US); Dennis J. Selkoe, Jamaica Plain, MA (US); Cuiman Cai, Brookline, MA (US)

(21) Appl. No.: 14/349,206

(22) PCT Filed: Oct. 4, 2012
(86) PCT No.: PCT/US12/58801

§ 371 Date: Apr. 2, 2014

Related U.S. Application Data
(60) Provisional application No. 61/543,287, filed on Oct. 4, 2011.

(51) Int. Cl. C07C 311/20; C07D 213/71; C07C 311/19; C07D 309/14; C07D 309/06; C07D 405/12; C07D 207/09

(52) U.S. Cl. CPC C07C 311/20; C07D 405/12; C07D 213/71; C07D 207/09; C07D 309/14; C07D 309/06; C07C 311/19
USPC 514/336; 514/347; 514/428; 514/459; 514/524; 514/539; 514/604; 546/282.1; 546/293; 548/567; 549/424; 549/426; 558/413; 560/12; 564/92

(57) ABSTRACT

This invention relates generally to the discovery of sulfonamide-containing compounds that are inhibitors of γ-secretase.

### CROSS REFERENCE TO RELATED APPLICATIONS

This application claims the benefit of U.S. Provisional Application No. 61/543,287, filed on Oct. 4, 2011, which is incorporated by reference in its entirety.

### TECHNICAL FIELD

This invention relates generally to the discovery of sulfonamide-containing compounds that are inhibitors of γ-secretase.

### BACKGROUND

Accumulating biochemical, histological, and genetic evidence supports the hypothesis that the 4 kDa β-amyloid protein (Aβ) is an essential component in the pathogenesis of Alzheimer's disease (“AD”). Selkoe D J, Science 275:630-631 (1997). Hardy J, Proc Natl Acad Sci USA 94:2095-2097 (1997). Despite the intense interest in the role of Aβ in the etiology of AD, the molecular mechanism of Aβ biosynthesis is still not fully understood. The 39-43-residue Aβ is formed via the sequential cleavage of the integral membrane amyloid precursor protein (APP) by (β- and γ-secretases. Selkoe D J, Annu Rev Cell Biol 10:373-403 (1994). β-Secretase cleavage of APP occurs near the membrane, producing the soluble APPg-β and a 12 kDa C-terminal membrane-associated fragment (CTF). The latter is processed by γ-secretase that cleaves within the transmembrane domain of the substrate to generate Aβ. An alternative proteolytic event carried out by α-secretase occurs within the Aβ portion of APP, releasing APPg-α. Subsequent processing of the resulting membrane-bound 10 kDa CTF by γ-secretase leads to the formation of a 3 kDa N-terminally truncated version of Aβ called p3.

Heterogeneous proteolysis of the 12 kDa CTF by γ-secretase generates primarily two C-terminal variants of Aβ, 40- and 42-amino acid versions (Aβ40 and Aβ42), and parallel processing of the 10 kDa CTF generates the corresponding C-terminal variants of p3. Although Aβ42 represents only about 10% of secreted Aβ, this longer and more hydrophobic variant is disproportionally present in the amyloid plaques observed post mortem in AD patients (Roher A E et al., Proc Natl Acad Sci USA 90:10836-40 (1993); Iwatsubo T et al., Neuron 13:45-53 (1994)) which is consistent with in vitro studies illustrating the kinetic insolubility of Aβ42 vis-a-vis Aβ40. Jarrett J T et al., Biochemistry 32:4693-4697 (1993). Importantly, all genetic mutations associated with early-onset (<60 years) familial Alzheimer's disease (FAD) result in increased Aβ42 production. Selkoe D J, Science 275:630-631 (1997); Hardy J, Proc Natl Acad Sci USA 94:2095-2097 (1997).

γ-secretase is therefore believed to be an attractive target for inhibitor design for the purpose of inhibiting production of Aβ and treating disorders characterized by the production and deposition of β-amyloid.

### SUMMARY

This invention relates generally to the discovery of sulfonamide-containing compounds that are inhibitors of γ-secretase.

As used herein, it should be appreciated that the term “inhibitor” refers to a compound that modulates (e.g., reduces) the activity of its target (e.g., protease) regardless of the mode of action of the inhibitor. Accordingly, in some embodiments, an inhibitor may react at the active site (e.g., catalytic site) of a protease thereby reducing its activity (e.g., inactivating the protease). In some embodiments, an inhibitor may be a transition state inhibitor. In some embodiments, an inhibitor may be a modulator (e.g., an allosteric modulator) that inhibits protease activity by binding to a modulatory site that indirectly alters the conformation of the active site, substrate binding site, or other site (or combination thereof) thereby modulating the activity of the protease (e.g., reducing the activity of the protease, changing the specificity of the protease, etc., or any combination thereof). In some embodiments, an inhibitor may modulate protease activity either by binding to the protease or to a substrate (or a combination thereof) thereby reducing the activity of the protease for the substrate. In some embodiments, an inhibitor may bind to the protease at a position that interferes with one or more substrate binding and/or product release steps. It should be appreciated that aspects of the invention are not limited by the precise mode of action of the inhibitor and that any direct or indirect effect on the activity of a protease may result from contacting γ-secretase with an inhibitor of the invention. In some embodiments, without wishing to be limited by theory, an inhibitor of the invention may bind to a proposed modulatory site on γ-secretase (see, e.g., Lazarov et. al., P.N.A.S., vol. 103, p. 6889). It also should be appreciated that an inhibitor of the invention may partially or completely inhibit the secretase activity (e.g., by about 10%, about 20%, about 30%, about 40%, about 50%, about 60%, about 70%, about 80%, about 90%, about 95%, or by less or more than any of these values, for example, by 100%, or by any intermediate percentage). In some embodiments, inhibition may be specific (e.g., substrate specific) in that the inhibitory effect is stronger for a first substrate than a second substrate. In some embodiments, specific inhibitors of the invention reduce degradation of the amyloid precursor protein to a greater extent than that of the Notch protein (e.g., the ratio of % inhibition of amyloid precursor protein degradation to % inhibition of Notch protein degradation is greater than 1). In some embodiments, amyloid precursor protein degradation by γ-secretase may be inhibited by a compound of the invention, whereas Notch degradation by γ-secretase may be unaffected or only slightly inhibited. Certain aspartyl proteases, including γ-secretase, generate β-amyloid from amyloid precursor protein (APP) which may result in neurodegenerative disorders. The γ-secretase inhibitor compounds are useful for treating a subject having or at risk of developing a neurodegenerative disorder associated with γ-secretase activity, e.g., Alzheimer's disease. In some aspects, specific inhibitors of the invention may be used to treat or prevent Alzheimer's disease without causing side effects associated with inhibition of Notch degradation.

The invention also features compositions (e.g., pharmaceutical compositions) and articles of manufacture that include one of more of the compounds described herein as well as methods of making, identifying, and using such compounds.

Other features and advantages are described in, or will be apparent from, the present specification.

Accordingly, in one aspect, compounds having formula (I) are featured:

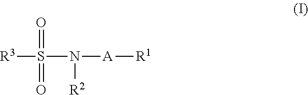

in which:

### R¹ is:

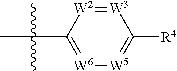

wherein:
W², W³, W⁵, and W⁶ are defined according to (A) or (B) below:
   (A)
   each of W² and W⁶ is independently selected from CH, C(halo), C(C₁-C₆ alkoxy) and C(C₁-C₆ haloalkoxy) (and optionally also C(OH)); and
   each of W³ and W⁵ is independently selected from CH; C(halo); C(C₁-C₆ alkoxy) (and optionally also C(OH)); and CR′; wherein R′ is —C(O)OH, —C(O)O(R⁴¹, e.g., C₁-C₆ alkyl), —C(O)NR⁴²R⁴³; or —CN; or
   (B)
   one or two of W², W³, W⁵, and W⁶ are N; and the others are independently selected from CH, C(halo), C(C₁-C₆ alkoxy), and C(C₁-C₆ haloalkoxy);
R⁴ is selected from any of the substituents delineated in (i)-(v) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
(iv) heteroaryl containing 5 ring atoms, wherein from 1-4 of the ring atoms is independently selected from N, NH, N(C₁-C₆ alkyl), O, and S; and wherein said heteroaryl is optionally substituted with from 1-3 independently selected R^{b}; or
(v) hydrogen;
R⁴¹ is C₁-C₈ alkyl, C₁-C₈ haloalkyl, or benzyl optionally substituted with from 1-3 R^{b};
each of R⁴² and R⁴³ is, independently:
(i) hydrogen; or
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 R^{c};
or
R⁴²—N—R⁴³ together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{c};
R⁴⁴ is hydrogen, C₁-C₈ alkyl, or C₁-C₈ haloalkyl;
R⁴⁵ is C₁-C₈ alkyl or C₁-C₈ haloalkyl;
   In embodiments, it is provided that only one of R⁴ and R′ or only one of R⁴ and two occurrences of R′ can be —C(O)OH, —C(O)O(R⁴¹, e.g., C₁-C₆ alkyl), —C(O)NR⁴²R⁴³; or —CN;
   A is C(R^{A})₂, wherein each occurrence of R^{A} is independently selected from hydrogen, fluoro, and —CH₃ (e.g., hydrogen, and —CH₃; e.g., hydrogen);

### R² is:

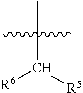 [0033] wherein R⁵ and R⁶ are defined according to (C) or (D) below:
   (C)
R⁵ and R⁶, together with the carbon atom to which each is attached, is C₃-C₈ cycloalkyl; or heterocyclyl containing from 3-8 ring atoms, wherein from 1-2 of the ring atoms is independently selected from N, NH, N(C₁-C₆ alkyl), O, and S; wherein each of said cycloalkyl and heterocyclyl is optionally substituted with from 1-5 R^{c}; or
   (D)
R⁵ is C₃-C₈ cycloalkyl; or heterocyclyl containing from 3-8 ring atoms, wherein from 1-2 of the ring atoms is independently selected from N, NH, N(C₁-C₆ alkyl), O, and S; wherein each of said cycloalkyl and heterocyclyl is optionally substituted with from 1-5 R^{c}; and
R⁶ is C₁-C₆ alkyl or C₁-C₆ haloalkyl, each of which is optionally substituted with a substituent selected from —OH, OCH₃, OCF₃, and —CN (in other embodiments, R⁶ can also be hydrogen);

### R³ is:

(i) C₆-C₁₀ aryl, which is optionally substituted with from 1-3 independently selected R^{d}; or
(ii) heteroaryl, each containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d};
R^{a} at each occurrence is, independently, selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN;
R^{b} at each occurrence is, independently selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —NH₂, —NH(C₁-C₆ alkyl), N(C₁-C₆ alkyl)₂, —NHC(O)(C₁-C₆ alkyl), —CN; and —NO₂;
R^{c} at each occurrence is independently selected from the substituents delineated in (aa), (bb) and (cc) below:
(aa) halo; C₁-C₆ alkoxy; C₁-C₆ haloalkoxy; C₁-C₆ thioalkoxy; C₁-C₆ thiohaloalkoxy; C₁-C₆ alkyl, C₁-C₆ haloalkyl, —NH(C₁-C₆ alkyl), N(C₁-C₆ alkyl)₂, —NHC(O)(C₁-C₆ alkyl), wherein the alkyl portion of each is optionally substituted with —OH, C₁-C₃ alkoxy, —C(O)OH, —C(O)O(C₁-C₆ alkyl), and —CN;
(bb) —OH; —CN; —NH₂; C₂-C₄ alkenyl; C₂-C₄ alkynyl; —C(O)H; —C(O)(C₁-C₆ alkyl); C(O)OH; —C(O)O(C₁-C₆ alkyl); —C(O)NH₂; —SO₂(C₁-C₆ alkyl); —SO₂(C₁-C₆ haloalkyl); —C(O)NR″′R″″—SO₂NR″′R″″, —SO₂NH₂, —NHCO(C₁-C₆ alkyl), —NHSO₂(C₁-C₆ alkyl), whereby R″′ and R″″ is independently selected from H, C₁-C₆ alkyl, C₁-C₆ haloalkyl;
(cc) C₃-C₆ cycloalkyl; or heterocyclyl or heterocyclyloxy, each containing from 5-6 ring atoms, wherein from 1-2 of the ring atoms of the heterocyclyl (or heterocyclyl portion) is independently selected from N, NH, N(C₁-C₆ alkyl), NC(O)(C₁-C₆ alkyl), O, and S; and wherein each of said cycloalkyl, heterocyclyl, and heterocyclyloxy is optionally substituted with from 1-3 substituents independently selected from —OH and C₁-C₄ alkyl;
   and
   R^{d} at each occurrence is, independently selected from halo, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN; COOH, NO₂, C(O)(C₁-C₆ alkyl), C(O)(C₁-C₆ haloalkyl), azido, NCS, —CH₂OH, amino, NR″′R″″, N-azidinyl, N-morpholinyl, S(C₁-C₆ alkyl), —SO₂(C₁-C₆ alkyl), —C(O)NR″′R″″—SO₂NR″′R″″, —SO₂NH₂, —NHCO(C₁-C₆ alkyl), —NHSO₂(C₁-C₆ alkyl), whereby R″′ and R″″ is independently selected from H, C₁-C₆ alkyl, C₁-C₆ haloalkyl;
or a pharmaceutically acceptable salt thereof.

In another aspect, any of the formula (I) compounds specifically described herein are featured.

In one aspect, compositions (e.g., a pharmaceutical composition) are featured which includes a compound of formula (I) (including any subgenera or specific compound thereof as described anywhere herein, including those in the claims) or a salt (e.g., a pharmaceutically acceptable salt) thereof as defined anywhere herein and a pharmaceutically acceptable carrier. In some embodiments, the compositions include an effective amount of the compound or salt. In some embodiments, the compositions can further include one or more additional therapeutic agents.

In one aspect, methods are featured for treating (e.g., controlling, relieving, ameliorating, alleviating, or slowing the progression of) or for preventing (e.g., delaying the onset of or reducing the risk of developing) a disease, disorder, or condition associated with γ-secretase activity. The methods include administering to a subject having (or at risk of having) the disease, disorder, or condition a therapeutically effective amount of a compound of formula (I) (including any subgenera or specific compound thereof as described anywhere herein, including those in the claims) or a salt (e.g., a pharmaceutically acceptable salt) thereof as defined anywhere herein, or a therapeutic preparation, composition, or formulation thereof.

For example, methods are featured for treating a neurodegenerative disorder (e.g., Alzheimer's disease) in a subject having, or at risk of having a neurodegenerative disorder, which comprises administering to the subject having, or at risk of having a neurodegenerative disorder a therapeutically effective amount of a compound of formula (I), or a pharmaceutically acceptable salt thereof.

As another example, a compound of formula (I), or a pharmaceutically acceptable salt thereof, for use in the treatment of a neurodegenerative disorder (e.g., Alzheimer's disease) is featured.

As a further example, a use of a compound of formula (I), or a pharmaceutically acceptable salt thereof, in the manufacture of a medicament for the treatment of a neurodegenerative disorder (e.g., Alzheimer's disease) is featured.

In still another example, a use of a compound of formula (I), or a pharmaceutically acceptable salt thereof, for use in therapy is featured.

In certain embodiments, the disease, disorder, or condition can be a neurodegenerative disorder, e.g., Alzheimer's disease.

In other embodiments, the subject can be a subject that has, or is at risk of developing, cancer. The cancer can be a gastrointestinal cancer (e.g., cancer of the esophagus, gallbladder, liver, pancreas, stomach, small intestine, large intestine, colon, or rectum). In some embodiments, the cancer can be leukemia or any solid tumors of which inhibition of γ-secretase can lead to therapeutic effects in cancer chemotherapy.

It should be appreciated that any one or more of the compounds of formula (I) may be used to inhibit γ-secretase activity by interaction with γ-secretase (e.g., in vitro or in vivo) with any one or more of the compounds. The invention also relates to methods of making medicaments for use in treating a subject, e.g., for treating a subject having a disease, disorder, or condition associated with γ-secretase activity, or at risk of developing disease, disorder, or condition associated with γ-secretase activity, treating a subject having Alzheimer's disease, or at risk of developing Alzheimer's disease, inhibiting APP cleavage, and/or inhibiting γ-secretase activity. Accordingly, one or more compounds or compositions described herein that inhibit γ-secretase activity as described herein may be used for the preparation of a medicament for use in any of the methods of treatment described herein. In some embodiments, the invention provides for the use of one or more compounds or compositions of the invention for the manufacture of a medicament or pharmaceutical for treating a mammal (e.g., a human) having one or more symptoms of, or at risk for, a disease or condition associated with γ-secretase activity (e.g., Alzheimer's disease).

In some embodiments, a compound of formula (I) (including any subgenera or specific compound thereof as described anywhere herein, including those in the claims) or a salt (e.g., a pharmaceutically acceptable salt) thereof as defined anywhere herein inhibits γ-secretase activity by at least 10% (e.g., by about 50%, by about 75%, by about 80%, by about 90%, by about 95%, or more, for example, completely inhibits) at a concentration of 1, 10 or 100 μM in an assay described herein (e.g., the γ-secretase assay). Accordingly, in some embodiments, a compound of the invention does not have less than 10% inhibitory activity when assayed at a concentration of about 1, 10 or 100 μM in an assay described herein (e.g., γ-secretase assay). In some embodiments, the inhibitory activity of a compound is selective for γ-secretase mediated cleavage of APP relative to the Notch protein. Accordingly, in some embodiments, a compound of the invention inhibits γ-secretase activity against APP (e.g., by at least 10%, by about 50%, by about 75%, by about 80%, by about 90%, by about 95%, or more, for example, completely inhibits) to a greater extent than it inhibits γ-secretase activity against the Notch protein. In some embodiments, a compound of the invention that inhibits APP cleavage does not inhibit Notch cleavage significantly (e.g., no inhibition of Notch cleavage, or enhanced Notch cleavage, is observed using an assay described herein, for example the N-100 assay or other assay). In some embodiments, an inhibitor is at least 5 fold (e.g., at least 10 fold, at least 100 fold, etc.) more selective for inhibiting APP cleavage relative to Notch cleavage. In certain embodiments, a compound of the invention has an IC₅₀ value of from about 28 nM to about 13 μM for APP (Aβ1-40) in the in vitro biochemical assay but a higher IC₅₀ value (e.g., from about 8 μM to about 30 μM) for Notch in the N-100 assay. In other embodiments, in cellular assays, a compound of the invention has an IC₅₀ value of from about 15 nM to about 500 nM for APP (Aβ40) and an IC₅₀ value of from about 1 nM to 100 nM for APP (Aβ42) was observed and a higher IC₅₀ value (e.g., 34 μM) as determined in a Notch cellular assay. However, it should be appreciated that a compound of the invention may be selective even if it has a higher IC₅₀ value for APP, provided that the IC₅₀ value for Notch is relatively higher.

In some embodiments, the subject can be in need thereof (e.g., a subject identified as being in need of such treatment, such as a subject having, or at risk of having, one or more of the diseases or conditions described herein). Identifying a subject in need of such treatment can be in the judgment of a subject or a health care professional and can be subjective (e.g., opinion) or objective (e.g., measurable by a test or diagnostic method). In some embodiments, the subject can be a mammal. In certain embodiments, the subject can be a human.

In some embodiments, abnormally high levels of γ-secretase activity imply statistically significantly higher levels (e.g., 10% higher, 20% higher, 30% higher, 50% higher, or higher) than a reference level characteristic of normal levels of activity.

However, it should be appreciated that AD patients or those at risk of developing AD may not necessarily have elevated levels of γ-secretase and/or elevated γ-secretase activity. Instead such subjects may suffer the effects of Aβ which is pathogenic and which can be produced by γ-secretase at all levels. In some embodiments, elevated levels of Aβ are pathogenic. Levels of Aβ depend on a balance between production and clearance. There are many factors that are involved in the production and clearance of Aβ. Accordingly, in some embodiments decreasing the γ-secretase-mediated production of Aβ can slow, halt and/or prevent the neurodegenerative effects of Aβ. Therefore, decreasing the γ-secretase production of Aβ (by up to 10%, or up to 20%, or up to 30%, or up to 40%, or up to 50%, or higher) relative to a baseline activity can yield a therapeutic effect and/or prevent disease onset and/or delay the onset of AD. It should be appreciated that γ-secretase activity in a subject can be measured from Aβ levels in plasma and cerebral spinal fluid (CSF). Accordingly, levels of Aβ inhibition can be assayed by measuring Aβ levels in the plasma and CSF with different compounds and comparing the levels to a reference level obtained without a test compound or using a compound that is known not to affect Aβ inhibition (e.g., a reference compound that is not a γ-secretase inhibitor). In some embodiments, compositions of the invention are administered to a patient that has, or is at risk of developing, Alzheimer's disease.

The term “subject having (or at risk of having) neurodegenerative disorders” (and the like) refers to a subject that is affected by or at risk of developing neurodegenerative disorders (e.g. predisposed, for example, genetically predisposed, to developing Alzheimer's disease) and/or any neurodegenerative disorders characterized by pathological aggregations of β-amyloid proteins or peptide fragments.

In one aspect, methods of making the pharmaceutical compositions described herein are featured. In embodiments, the methods include taking any one or more of the compounds of formula (I) (including any subgenera or specific compound thereof as described anywhere herein, including those in the claims) or a salt (e.g., a pharmaceutically acceptable salt) thereof as defined anywhere herein, and mixing said compound(s) with one or more pharmaceutically acceptable carriers.

In one aspect, kits for treating (e.g., controlling, relieving, ameliorating, alleviating, or slowing the progression of) or for preventing (e.g., delaying the onset of or reducing the risk of developing) a disease, disorder, or condition associated with γ-secretase activity, e.g., a neurodegenerative disorder, e.g., Alzheimer's disease, in a subject are featured. The kits include (i) a compound of formula (I) (including any subgenera or specific compound thereof as described anywhere herein, including those in the claims) or a salt (e.g., a pharmaceutically acceptable salt) thereof as defined anywhere herein; and (ii) instructions that include a direction to administer said compound to a subject (e.g., a patient). In a preferred embodiment the subject is a human. In some embodiments, an article of manufacture may include two or more compounds or compositions of the invention alone or along with one or more additional compounds or compositions that are useful for treating Alzheimer's disease as described herein.

In another aspect, methods of making the compounds described herein are featured. In embodiments, the methods include taking any one of the intermediate compounds described herein and reacting it with one or more chemical reagents in one or more steps to produce a compound of formula (I) (including any subgenera or specific compound thereof as described anywhere herein, including those in the claims) or a salt (e.g., a pharmaceutically acceptable salt) thereof as defined anywhere herein.

In embodiments, any compound, composition, or method described herein can also include any one or more of the other features delineated in the detailed description and/or in the claims.

For example, embodiments can include any one or more of the following features.

W², W³, W⁵, and W⁶ can be defined according to definition (A).

Each of W³ and W⁵ can be independently selected from CH, C(halo), and C(C₁-C₆ alkoxy).

Each of W², W³, W⁵, and W⁶ can be independently selected from CH and C(halo).

Each of W², W³, W⁵, and W⁶ can be CH.

One of W³ and W⁵ can be CR′, and the other of W³ and W⁵ is CH, C(halo), or C(C₁-C₆ alkoxy).

One of W³ and W⁵ can be CR′, and the other of W³ and W⁵ is CH.

Each of W² and W⁶ can be independently selected from CH and C(halo) (e.g., each of W² and W⁶ is CH).

R′ can be —C(O)OH or —C(O)O(C₁-C₆ alkyl) (e.g., R′ is —C(O)OH).

W², W³, W⁵, and W⁶ can be defined according to definition (B).

One or two of W³ and W⁵ can be N.

One of W³ and W⁵ can be N; the other of W³ and W⁵ can be independently selected from CH or C(halo) (e.g., the other of W³ and W⁵ is CH); and each of W² and W⁶ can be independently selected from CH and C(halo) (e.g., each of W² and W⁶ is CH).

R⁴ can be selected from halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, C₁-C₆ alkoxy, and —SO₂ (R⁴⁵)

R⁴ can be selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵).

R⁴ can be —CO₂H.

R⁴ can be —CO₂R⁴¹.

R⁴¹ can be C₁-C₈ alkyl (e.g., R⁴¹ can be CH₃).

R⁴ can be —SO₂(R⁴⁵).

R⁴⁵ can be C₁-C₈ alkyl (e.g., R⁴⁵ can be CH₃).

R⁴ can be —C(O)N(R⁴²)(R⁴³).

Each of R⁴² and R⁴³ is independently selected from: (i) hydrogen; and (ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

One of R⁴² and R⁴³ can be hydrogen; and the other of R⁴² and R⁴³ can be C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

One of R⁴² and R⁴³ can be hydrogen; and the other of R⁴² and R⁴³ can be C₁-C₈ alkyl, which is optionally substituted with from 1-3 (e.g., 1) R^{c}.

R^{c} at each occurrence can be, independently, —OH; C₁-C₆ alkoxy (e.g., OCH₃); —C(O)(C₁-C₆ alkyl) (e.g., —C(O)CH₃); or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein said heterocyclyl can be optionally substituted with from 1-3 substituents independently selected from —OH and C₁-C₄ alkyl (e.g., pyranyl).

One of R⁴² and R⁴³ can be hydrogen; and the other of R⁴² and R⁴³ can be C₃-C₈ cycloalkyl; or heterocyclyl containing from 3-8 ring atoms, wherein from 1-2 of the ring atoms can be independently selected from N, NH, N(C₁-C₆ alkyl), O, and S; and wherein each of said cycloalkyl or heterocyclyl can be optionally substituted with from 1-3 (e.g., 1) R^{c} (e.g., —OH).

R⁴²—N—R⁴³ together can form a saturated ring having 5 or 6 ring atoms, in which from 1 or 2 ring atoms, in addition to the N that occurs between R⁴² and R⁴³, can be optionally a heteroatom independently selected from NH, N(alkyl), O, or S; and wherein said saturated ring can be optionally substituted with from 1-3 R^{c} (e.g., R⁴²—N—R⁴³ together can form a morpholino ring).

R⁴ can be heterocyclyloxy, each containing from 3-8 ring atoms, wherein from 1-2 of the ring atoms can be independently selected from N, NH, N(C₁-C₆ alkyl), O, and S; and wherein said heterocyclyloxy can be optionally substituted with from 1-3 independently selected R^{a} (e.g., pyranyloxy).

R⁵ and R⁶ can be defined according to (C).

R⁵ and R⁶, together with the carbon atom to which each is attached, can be C₃-C₈ cycloalkyl, which is optionally substituted with from 1-5 R^{c}.

R⁵ and R⁶, together with the carbon atom to which each is attached, can be C₃-C₆ (e.g., C₅-C₆) cycloalkyl, which can be optionally substituted with from 1-5 R^{c}.

R⁵ and R⁶, together with the carbon atom to which each is attached, can be C₆ cycloalkyl, which can be optionally substituted with from 1-5 R^{c}.

R^{c} at each occurrence can be, independently, —OH or C₁-C₆ alkyl (e.g., CH₃).

R⁵ and R⁶ can be defined according to (D).

R⁵ can be C₃-C₈ (e.g., C₃-C₆, C₅-C₆, C₆) cycloalkyl, which can be optionally substituted with from 1-5 R^{c} (e.g., —OH or C₁-C₆ alkyl, such as CH₃).

R⁶ can be C₁-C₆ alkyl, which can be optionally substituted with a substituent selected from —OH and —CN (e.g., —OH).

R⁶ can be —CH₂CH₃.

R⁶ can be —CH₃.

The carbon attached to R⁵ and R⁶ can have the S configuration.

R³ can be C₆-C₁₀ aryl, which is optionally substituted with from 1-3 independently selected R^{d}.

R³ can be phenyl that is substituted with 1 or 2 independently selected R^{d}.

R³ can be 4-chloro-phenyl, 4-fluoro-phenyl, or 2,4-difluorophenyl (e.g., 4-chloro-phenyl).

R³ can be heteroaryl containing from 5-10 ring atoms, wherein from 1-6 of the ring atoms can be independently selected from N, NH, N(C₁-C₆ alkyl), O, and S; and wherein said heteroaryl ring is optionally substituted with from 1-3 independently selected R^{d}.

R³ can be heteroaryl containing from 5-6 ring atoms, wherein from 1-4 of the ring atoms is independently selected from N, NH, N(C₁-C₆ alkyl), O, and S; and wherein said heteroaryl ring is substituted with 1 or 2 independently selected R^{d}.

R³ can be thienyl, which is substituted with 1 or 2 independently selected R^{d}.

R^{d} at each occurrence is independently selected from halo.

A can be CH₂.

The compound can be any one or more of the title compounds of Examples 1-90.

Embodiments can include any one or more of the following advantages.

Some of the compounds of formula (I) selectively inhibit γ-secretase-mediated cleavage of APP with little or no inhibition of the γ-secretase-mediated cleavage of the Notch family of transmembrane receptors. Selective inhibition of the cleavage of APP relative to that of the Notch receptor is believed to minimize certain unwanted side effects, such as lymphopoiesis and intestinal cell differentiation.

Some of the compounds of formula (I) exhibit enhanced solubility in aqueous media. For example, some of the compounds of formula (I) (e.g., compounds in which R⁴ is other than hydrogen, e.g., compounds in which R⁴ is C(O)OH) exhibit a solubility that is 305 μM in a PBS buffer at pH 7.4. In embodiments, the compounds described herein exhibited a range of solubility from about 0.1 μM to about 260 μM in PBS at pH 7.4.

### DEFINITIONS

The term “mammal” includes organisms, which include mice, rats, cows, sheep, pigs, rabbits, goats, horses, monkeys, dogs, cats, and humans.

“An effective amount” refers to an amount of a compound that confers a therapeutic effect (e.g., treats, controls, relieves, ameliorates, alleviates, or slows the progression of); or prevents, e.g., delays the onset of or reduces the risk of developing, a disease, disorder, or condition or symptoms thereof on the treated subject. The therapeutic effect may be objective (i.e., measurable by some test or marker) or subjective (i.e., subject gives an indication of or feels an effect). For example, disease progression can be monitored by clinical observations, laboratory and neuroimaging investigations apparent to a person skilled in the art. The effective amount of any one or more compounds may be from about 10 ng/kg of body weight to about 1,000 mg/kg of body weight, and the frequency of administration may range from once a day to once a week. However, other dosage amounts and frequencies also may be used as the invention is not limited in this respect. It should be appreciated that one or more compounds and/or compositions of the invention may be used alone or in combination with one or more additional compounds or compositions to treat a subject that has Alzheimer's disease or that is at risk of developing Alzheimer's disease. In some embodiments, an additional compound may be an alternative inhibitor of β-amyloid production. In some embodiments, an additional compound can be a β-secretase inhibitor. In some embodiments, an additional compound may be a compound that is therapeutically useful for treating Alzheimer's disease or symptoms thereof (e.g., an acetyl-cholinesterase inhibitor, for example, Aricept; an anti-depressive agent, for example, rivastigmine; or any combination thereof). A combination therapy may involve combining one or more (e.g., 1, 2, 3, 4, 5, 6, 7, 8, 9, 10, or more) compounds of the invention with one or more (e.g., 1, 2, 3, 4, 5, 6, 7, 8, 9, 10, or more) additional compounds described herein. It should be appreciated that combination therapies may include compositions comprising of one or more compounds and/or administering one or more compounds in combination (e.g., together or separately, but according to a coordinated regimen, etc.). It should be appreciated that compounds or compositions of the invention may be administered in an amount effective to treat a neurological disorder such as Alzheimer's disease in a subject. In some embodiments, a treatment may prevent the onset or development of disease or disease symptoms in a subject at risk of the disease (e.g., in a subject with a family history of Alzheimer's, a subject with early symptoms of Alzheimer's, a subject of an age associated with a higher risk for Alzheimer's, a subject with any other risk factor for Alzheimer's, or a subject with any combination of two or more risk factors described herein). In some embodiments, a treatment may prevent or reduce the progression of the disease in a subject diagnosed as having Alzheimer's disease. In some embodiments, a treatment may promote disease regression. In preferred embodiments, the subject is a human.

Effective doses will also vary depending on route of administration, as well as the possibility of co-usage with other agents. A therapeutically effective amount can be an amount that is effective in a single dose or an amount that is effective as part of a multi-dose therapy, for example, an amount that is administered in two or more doses or an amount that is administered chronically.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (Here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, and polycyclic rings.

The following definitions are used unless otherwise described. Specific and general values listed below for radicals, substituents, and ranges are for illustration only. They do not exclude other defined values or other values within defined ranges for the radicals and substituents. Unless otherwise indicated, alkyl, alkoxy, alkenyl, and the like denote both straight and branched groups.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substitutents. Examples of alkyl groups include, without limitation, methyl, ethyl, n-propyl, isopropyl, and tert-butyl.

The term “haloalkyl” refers to an alkyl group in which at least one hydrogen atom is replaced by halo. In some embodiments, more than one hydrogen atom (e.g., 2, 3, 4, 5, 6, 7, 8, 9, 10, 11, 12, 13, or 14) is replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents.

As referred to herein, the term “alkoxy” refers to a group of formula —O(alkyl). Alkoxy can be, for example, methoxy (—OCH₃), ethoxy, propoxy, isopropoxy, butoxy, iso-butoxy, sec-butoxy, tert-butoxy, pentoxy, 2-pentoxy, 3-pentoxy, or hexyloxy. Likewise, the term “thioalkoxy” refers to a group of formula —S(alkyl). The terms “haloalkoxy” and “thio-haloalkoxy” refer to —O(haloalkyl) and —S(haloalkyl), respectively. Finally, the term “heterocyclyloxy” refers to a group of the formula —O(heterocyclyl).

The term “alkenyl” refers to a straight or branched hydrocarbon chain containing the indicated number of carbon atoms and having one or more carbon-carbon double bonds. Any atom can be optionally substituted, e.g., by one or more substituents. Alkenyl groups can include, e.g., vinyl, allyl, 1-butenyl, and 2-hexenyl. One of the double bond carbons can optionally be the point of attachment of the alkenyl substituent.

The term “alkynyl” refers to a straight or branched hydrocarbon chain containing the indicated number of carbon atoms and having one or more carbon-carbon triple bonds. Alkynyl groups can be optionally substituted, e.g., by one or more substituents. Alkynyl groups can include groups such as ethynyl, propargyl, and 3-hexynyl. One of the triple bond carbons can optionally be the point of attachment of the alkynyl substituent.

The term “heterocyclyl” refers to a fully saturated monocyclic, bicyclic, tricyclic or other polycyclic ring system having one or more constituent heteroatom ring atoms independently selected from O, N (it is understood that one or two additional groups may be present to complete the nitrogen valence and/or form a salt), or S. The heteroatom or ring carbon can be the point of attachment of the heterocyclyl substituent to another moiety. Any atom can be optionally substituted, e.g., by one or more substituents. Heterocyclyl groups can include groups such as tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms”, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected R^{a} would include (but not be limited to) tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from O, N (it is understood that one or two additional groups may be present to complete the nitrogen valence and/or form a salt), or S. A ring carbon (e.g., saturated or unsaturated) or heteroatom can be the point of attachment of the heterocycloalkenyl substituent. Any atom can be optionally substituted, e.g., by one or more substituents. Heterocycloalkenyl groups can include groups such as dihydropyridyl, tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon group. Any atom can be optionally substituted, e.g., by one or more substituents. A ring carbon serves as the point of attachment of a cycloalkyl group to another moiety. Cycloalkyl moieties can include groups such as cyclopropyl, cyclobutyl, cyclopentyl, cyclohexyl, cycloheptyl, adamantyl, and norbornyl (bicyclo[2.2.1]heptyl).

The term “aryl” refers to an aromatic monocyclic, bicyclic (2 fused rings), tricyclic (3 fused rings), or polycyclic (>3 fused rings) hydrocarbon ring system. One or more ring atoms can be optionally substituted by one or more substituents for example. Aryl moieties include groups such as phenyl and naphthyl.

The term “heteroaryl” refers to an aromatic monocyclic, bicyclic (2 fused rings), tricyclic (3 fused rings), or polycyclic (>3 fused rings) hydrocarbon group having one or more heteroatom ring atoms independently selected from O, N (it is understood that one or two additional groups may be present to complete the nitrogen valence and/or form a salt), or S. One or more ring atoms can be optionally substituted, e.g., by one or more substituents. Examples of heteroaryl groups include, but are not limited to, 2H-pyrrolyl, 3H-indolyl, 4H-quinolizinyl, benzo[b]thienyl, furyl, imidazolyl, imidazolyl, indazolyl, indolyl, isoxazolyl, oxazolyl, perimidinyl, pyridyl, pyrimidinyl, pyrrolyl, quinazolinyl, quinolyl, quinoxalinyl, thiadiazolyl, thianthrenyl, thiazolyl, thienyl, and triazolyl.

As used herein, the descriptor “—CN” represents the cyano group, wherein the carbon and nitrogen atoms are bound together by a triple bond. As used herein, the descriptor “—OH” represents the hydroxy group. The descriptors “C═O” or “C(O)” refers to a carbon atom that is doubly bonded to an oxygen atom.

In general, when a definition for a particular variable includes hydrogen and non-hydrogen (halo, alkyl, aryl, etc.) possibilities, the term “substituent(s) other than hydrogen” refers collectively to the non-hydrogen possibilities for that particular variable.

The term “substituent” refers to a group “substituted” on groups such as an alkyl, haloalkyl, cycloakyl, heterocyclyl, aryl, or heteroaryl group at any atom of that group. In one aspect, the substituent(s) on a group are independently any one single or any combination of two or more of the permissible atoms or groups of atoms delineated for that substituent. In another aspect, a substituent may itself be substituted with any one of the above substituents.

Further, as used herein, the phrase “optionally substituted” means unsubstituted (e.g., substituted with hydrogen (H)) or substituted. As used herein, the term “substituted” means that a hydrogen atom is removed and replaced by a substituent. It is understood that substitution at a given atom is limited by valency.

Descriptors such as “C₆-C₁₀ aryl that is optionally substituted with from 1-4 independently selected R^{c} (and the like) is intended to include both an unsubstituted C₆-C₁₀ aryl group and a C₆-C₁₀ aryl group that is substituted with from 1-4 independently selected R^{c}. The use of a substituent (radical) prefix name such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

The details of one or more embodiments of the invention are set forth in the description below. Other features and advantages of the invention will be apparent from the description and from the claims.

### DETAILED DESCRIPTION

This invention relates generally to the discovery of sulfonamide-containing compounds that are inhibitors of γ-secretase.

In one aspect, compounds having formula (I) are featured:

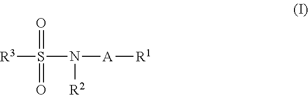

Here and throughout this specification, R¹, R², R³, and A can be as defined anywhere herein.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

Thus, for ease of exposition, it is also understood that where in this specification, a variable (e.g., R¹) is defined by “as defined anywhere herein” (or the like), the definitions for that particular variable include the first occurring and broadest generic definition as well as any sub-generic and specific definitions delineated anywhere in this specification.

Variable R¹

As defined above, R¹ has the following formula:

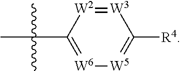

Variables W², W³, W⁵, and W⁶

In some embodiments, W², W³, W⁵, and W⁶ are defined according to (A) below:
(A)
each of W² and W⁶ is independently selected from CH, C(halo), C(C₁-C₆ alkoxy) and C(C₁-C₆ haloalkoxy) (and optionally also C(OH)); and
each of W³ and W⁵ is independently selected from CH; C(halo); C(C₁-C₆ alkoxy) (and optionally also C(OH)); and CR′; wherein R′ is —C(O)OH, —C(O)O(R⁴¹, e.g., C₁-C₆ alkyl), —C(O)NR⁴²R⁴³; or —CN.

In these embodiments, R¹ is an optionally substituted phenyl group.

In certain embodiments, each of W² and W⁶ is independently selected from CH and C(halo); e.g., each of W³ and W⁵ is CH.

In certain embodiments, each of W³ and W⁵ is other than CR′; e.g., each of W³ and W⁵ is independently selected from CH, C(halo), and C(C₁-C₆ alkoxy); e.g., each of W³ and W⁵ is independently selected from CH and C(halo); e.g., each of W³ and W⁵ is CH.

In certain embodiments, each of W², W³, W⁵, and W⁶ is independently selected from CH and C(halo).

In certain embodiments, each of W², W³, W⁵, and W⁶ is CH.

In certain embodiments, one of W³ and W⁵ is CR′, and the other of W³ and W⁵ is CH, C(halo), C(C₁-C₆ alkoxy).

Embodiments can include one or more of the following features.

The other of W³ and W⁵ is CH or C(halo); or the other of W³ and W⁵ is CH.

Each of W² and W⁶ is independently selected from CH and C(halo) (e.g., each of W² and W⁶ is CH).

The other of W³ and W⁵ is CH, and each of W² and W⁶ is CH.

R′ is —C(O)OH or —C(O)O(C₁-C₆ alkyl) or —C(O)NR⁴²R⁴³. R′ is —C(O)OH) or —C(O)NR⁴²R⁴³.

In some embodiments, W², W³, W⁵, and W⁶ are defined according to definition (B):
one or two of W², W³, W⁵, and W⁶ are N; and the others are independently selected from CH, C(halo), C(C₁-C₆ alkoxy) and C(C₁-C₆ haloalkoxy).

In certain embodiments, one or two of W², W³, W⁵, and W⁶ are N; and the others are independently selected from CH or C(halo).

In certain embodiments, one or two of W², W³, W⁵, and W⁶ are N; and the others are CH.

In certain embodiments, one or two of W³ and W⁵ is/are N.

For example, one of W³ and W⁵ is N; the other of W³ and W⁵ is independently selected from CH or C(halo) (e.g., the other of W³ and W⁵ is CH); and each of W² and W⁶ is independently selected from CH and C(halo) (e.g., each of W² and W⁶ is CH).

As another example, each of W³ and W⁵ is N; and one of W² and W⁶ is CH and the other of W² and W⁶ is C(halo). In certain embodiments, each of W² and W⁶ is CH.

In certain embodiments, one of W² and W³ is N; and the others are independently selected from CH or C (halo). In certain embodiments, one of W² and W³ is N; and the others are CH.

In certain of the above described embodiments (for both (A) and (B)), each occurrence of C(halo) is CF (in which F represents fluoro).

Variable R⁴

In some embodiments, R⁴ is selected from any of the substituents delineated in (i)-(iv) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
(iv) heteroaryl containing 5 ring atoms, wherein from 1-4 of the ring atoms is independently selected from N, NH, N(C₁-C₆ alkyl), O, and S; and wherein said heteroaryl is optionally substituted with from 1-3 independently selected R^{b};

In certain embodiments, R⁴ is selected from (i), (ii), and (iii) above.

In certain embodiments, R⁴ is selected from any of the substituents delineated in (i)-(iii) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyloxy, containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a}.

In embodiments, each of (i), (ii), (iii), and (iv) above can be any subset of substituents as defined anywhere herein.

In some embodiments, R⁴ is halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; or OCH(CH₂OH)₂.

In certain embodiments, R⁴ is selected from halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, C₁-C₆ alkoxy, and —SO₂(R⁴⁵).

In certain embodiments, R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵).

In some embodiments, R⁴ is —CO₂H.

In some embodiments, R⁴ is —C(O)OR⁴¹. In embodiments, R⁴¹ is C₁-C₈ alkyl (e.g., C₁-C₃ alkyl, e.g., CH₃ or CH₂CH₃; or C₃-C₆ alkyl, e.g., C₃-C₆ branched alkyl, e.g., t-butyl, isopropyl, isobutyl).

In some embodiments, R⁴ is —SO₂(R⁴⁵). In embodiments, R⁴⁵ is C₁-C₈ alkyl (e.g., C₁-C₃ alkyl, e.g., CH₃) or C₁-C₈ alkyl (e.g., C₁-C₃ haloalkyl, e.g., CF₃).

In some embodiments, R⁴ is —C(O)N(R⁴²)(R⁴³).

In certain embodiments, each of R⁴² and R⁴³ is independently selected from:
(i) hydrogen;
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

In certain embodiments, one of R⁴² and R⁴³ is hydrogen; and the other of R⁴² and R⁴³ is C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

In certain embodiments, one of R⁴² and R⁴³ is hydrogen; and the other of R⁴² and R⁴³ is C₁-C₈ alkyl, which is optionally substituted with from 1-3 (e.g., 1) R^{c}.

In embodiments, R^{c} at each occurrence is, independently, —OH; C₁-C₆ alkoxy (e.g., OCH₃); C₁-C₆ haloalkoxy (e.g., OCF₃); —C(O)(C₁-C₆ alkyl) (e.g., —C(O)CH₃); or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein said heterocyclyl is optionally substituted with from 1-3 substituents independently selected from —OH and C₁-C₄ alkyl (e.g., R^{c} can be pyranyl, e.g., 4-pyranyl).

In embodiments, one of R⁴² and R⁴³ is hydrogen, and the other of R⁴² and R⁴³ is C₁-C₈ alkyl or C₁-C₈ haloalkyl, each of which optionally substituted with —OH (e.g., C₁-C₈ alkyl, which is optionally substituted with —OH). For example, one of R⁴² and R⁴³ is hydrogen, and the other of R⁴² and R⁴³ is C₁-C₈ (e.g., C₁-C₆) alkyl which is substituted with —OH. For example, R⁴ can be CONHCH₂CH₂OH, CONHCH₂(CH₂)ₘOH, or CONHCH(CH₃)(CH₂)ₘOH, in which m is, independently, 1, 2, or 3. In embodiments, when R⁴ is CONHCH(CH₃)(CH₂)ₘOH (e.g., m=1), the carbon attached to CH₃ has the R-configuration.

In certain embodiments, one of R⁴² and R⁴³ is hydrogen; and the other of R⁴² and R⁴³ is C₃-C₈ (e.g., C₃-C₆, e.g., C₅-C₆) cycloalkyl; or heterocyclyl containing from 3-8 (e.g., 3-6, 5-6) ring atoms, wherein from 1-2 of the ring atoms is independently selected from N, NH, N(C₁-C₆ alkyl), O, and S; and wherein each of said cycloalkyl or heterocyclyl is optionally substituted with from 1-3 (e.g., 1) R^{c} (e.g., R^{c} is —OH). For example, the other of R⁴² and R⁴³ can be optionally substituted (e.g., R^{c} is —OH, OCH₃ or OCF₃) cyclopentyl or cyclohexyl (e.g., e.g., R^{c} is —OH; e.g., the hydroxylated ring carbon having the R-configuration or the S-configuration); or optionally substituted pyranyl (e.g., 4-pyranyl).

In certain embodiments, R⁴²—N—R⁴³ together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{c} (e.g., R⁴²—N—R⁴³ together forms a morpholino ring).

In some embodiments, R⁴ is heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a}.

In certain embodiments, R⁴ is heterocyclyl containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}. For example, R⁴ can be morpholino (e.g., 4-morpholino, pyrrolidine, piperidine, piperazine).

In certain embodiments, R⁴ is heterocyclyloxy, each containing from 3-8 ring atoms, wherein from 1-2 of the ring atoms is independently selected from N, NH, N(C₁-C₆ alkyl), O, and S; and wherein said heterocyclyloxy is optionally substituted with from 1-3 independently selected R^{a} (e.g., R⁴ can be pyranyloxy, e.g., 4-pyranyloxy; or the heterocyclyl portion can be as defined above).

In some embodiments, R⁴ is C₁-C₆ alkoxy, C₁-C₆ thioalkoxy, C₁-C₆ haloalkoxy, C₁-C₆ halothioalkoxy, C₁-C₆ alkyl, or C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

In certain embodiments, R⁴ is C₁-C₆ alkoxy, C₁-C₆ thioalkoxy, C₁-C₆ haloalkoxy, or C₁-C₆ halothioalkoxy, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

In certain embodiments, R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN. For example, R⁴ can be —OCH₃.

In certain embodiments, R⁴ is C₁-C₆ thioalkoxy or C₁-C₆ halothioalkoxy (e.g., C₁-C₆ thioalkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN. For example, R⁴ can be —SCH₃.

Non-Limiting Combinations of Variables W², W³, W⁵, and W⁶, and R⁴

In some of the above-described R⁴ embodiments, W², W³, W⁵, and W⁶ are defined according to definition (A) as defined anywhere herein. Non-limiting examples of W², W³, W⁵, and W⁶ include:
each of W², W³, W⁵, and W⁶ is CH; and
one of W³ and W⁵ is CR′, and the other of W³ and W⁵ is CH, and each of W² and W⁶ is CH.

In certain embodiments, each of W², W³, W⁵, and W⁶ is CH; and R⁴ is —CO₂H; —C(O)OR⁴¹; —C(O)N(R⁴²)(R⁴³); —SO₂(R⁴⁵), or heterocyclyloxy.

In certain embodiments, each of W², W³, W⁵, and W⁶ is CH; and R⁴ is —CO₂H; —C(O)OR⁴¹; —C(O)N(R⁴²)(R⁴³); or —SO₂(R⁴⁵).

In certain embodiments, each of W², W³, W⁵, and W⁶ is CH; and R⁴ is —CO₂H.

In certain embodiments, one of W³ and W⁵ is CR′ (e.g., CCO₂H) and the other of W³ and W⁵ is CH, and each of W² and W⁶ is CH, and R⁴ can be, e.g., H or C₁-C₆ alkoxy (e.g., OCH₃).

In some of the above-described R⁴ embodiments, W², W³, W⁵, and W⁶ are defined according to definition (B) as defined anywhere herein.

In some embodiments, one or more of the following (a) through (h) can apply:

(a) R⁴ is other than hydrogen.

(b) R⁴ is other than halo.

(c) R⁴ is other than C₁-C₆ alkoxy, C₁-C₆ thioalkoxy, C₁-C₆ haloalkoxy, or C₁-C₆ halothioalkoxy, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

(d) R⁴ is other than hydrogen, halo, C₁-C₆ alkoxy, C₁-C₆ thioalkoxy, C₁-C₆ haloalkoxy, or C₁-C₆ halothioalkoxy, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

(e) R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;

and

W², W³, W⁵, and W⁶ are defined according to definition (A);

and

one of W³ and W⁵ is CR′ (e.g., R′ is —C(O)OH or —C(O)O(C₁-C₆ alkyl); e.g., —C(O)OH).

(f) In certain embodiments, it is provided that when R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN; then W², W³, W⁵, and W⁶ are defined according to definition (A); and one of W³ and W⁵ is CR′ (e.g., —C(O)OH or —C(O)O(C₁-C₆ alkyl); e.g., —C(O)OH).

(g) R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;

and

W², W³, W⁵, and W⁶ are defined according to definition (A);

and

one or more of (or two or more of) W², W³, W⁵, and W⁶ is independently selected from C(halo (e.g., CF).

(h) In certain embodiments, it is provided that when R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN; and W², W³, W⁵, and W⁶ are defined according to definition (A); and one or more of (or two or more of) W², W³, W⁵, and W⁶ is independently selected from C(halo (e.g., CF).

Variable A

In some embodiments, A is CH₂ (i.e., each of R^{A} is hydrogen). In other embodiments, A is CF₂ (i.e., each of R^{A} is fluoro).

Variable R²

As defined above R² has the following formula:

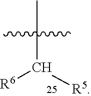

In some embodiments, R⁵ and R⁶ are defined according to (C):
(C)
R⁵ and R⁶, together with the carbon atom to which each is attached, is C₃-C₈ cycloalkyl; or heterocyclyl containing from 3-8 ring atoms, wherein from 1-2 of the ring atoms is independently selected from N, NH, N(C₁-C₆ alkyl), O, and S; wherein each of said cycloalkyl and heterocyclyl is optionally substituted with from 1-5 R^{c};

In certain embodiments, R⁵ and R⁶, together with the carbon atom to which each is attached, is C₃-C₈ (e.g., C₃-C₆, e.g., C₅-C₆) cycloalkyl, which is optionally substituted with from 1-5 (e.g., 1-4-1-3, 1-2, 1) R^{c}.

In certain embodiments, R^{c} at each occurrence is, independently, —OH or C₁-C₆ alkyl (e.g., CH₃) or C₁-C₆ haloalkyl (e.g., CF₃).

In certain embodiments, R⁵ and R⁶, together with the carbon atom to which each is attached, is C₆ cycloalkyl, which is optionally substituted with from 1-5 (e.g., 1-4-1-3, 1-2, 1) R^{c}, in which R^{c} can be as defined anywhere herein (e.g., —OH or C₁-C₆ alkyl, e.g., CH₃). For example, R⁵ and R⁶, together with the carbon atom to which each is attached, can be 2-methylcyclohexyl or 2,2,6,6-tetramethylcyclohexyl. In embodiments, the cyclohexyl ring can contain one more stereogenic centers, each of which independently having the R-configuration or the S-configuration.

In some embodiments, R⁵ and R⁶ are defined according to (D):
(D)
R⁵ is C₃-C₈ cycloalkyl; or heterocyclyl containing from 3-8 ring atoms, wherein from 1-2 of the ring atoms is independently selected from N, NH, N(C₁-C₆ alkyl), O, and S; wherein each of said cycloalkyl and heterocyclyl is optionally substituted with from 1-5 R^{c}; and
R⁶ is C₁-C₆ alkyl or C₁-C₆ haloalkyl, each of which is optionally substituted with a substituent selected from —OH, OCH₃, OCF₃, and —CN.

In certain embodiments, R⁵ is C₃-C₈ (e.g., C₃-C₆, C₅-C₆, C₆) cycloalkyl, which is optionally substituted with from 1-5 (e.g., 1-4-1-3, 1-2, 1) R^{c} (e.g., —OH or C₁-C₆ alkyl, such as CH₃).

In certain embodiments, R⁶ is C₁-C₆ alkyl, which is optionally substituted with a substituent selected from —OH and —CN (e.g., —OH). For example, R⁶ can be —CH₂CH₃ or —CH₃.

In certain embodiments, R⁵ is C₃-C₈ (e.g., C₃-C₆, C₅-C₆, C₆) cycloalkyl, which is optionally substituted with from 1-5 (e.g., 1-4-1-3, 1-2, 1) R^{c} (e.g., —OH or C₁-C₆ alkyl, such as CH₃) and R⁶ is C₁-C₆ alkyl, which is optionally substituted with a substituent selected from —OH and —CN (e.g., —OH). For example, R⁶ can be —CH₂CH₃ or —CH₃.

In certain embodiments, the carbon attached to R⁵ and R⁶ has the S configuration.

Variable R³

In some embodiments, R³ is C₆-C₁₀ aryl, which is optionally substituted with from 1-3 independently selected R^{d}.

In embodiments, R^{d} at each occurrence is independently selected from halo (e.g., fluoro or chloro).

In certain embodiments, R³ is C₆-C₁₀ aryl, which is substituted with from 1-3 independently selected R^{d}, in which R^{d} can be as defined anywhere herein.

In certain embodiments, R³ is phenyl, which is substituted with from 1-3 independently selected R^{d}, in which R^{d} can be as defined anywhere herein.

In certain embodiments, R³ is phenyl that is substituted with 1 or 2 (e.g., 1) R^{d}, in which R^{d} can be as defined anywhere herein. In certain embodiments, R^{d} or at least one R^{d} is attached to the phenyl ring carbon that is para with respect to the phenyl ring carbon that is attached to the sulfur atom of the sulfonyl group. For example, R³ can be 4-chloro-phenyl, 4-fluoro-phenyl, or 2,4-difluorophenyl. In certain embodiments, R^{d} or at least one R^{d} is attached to the phenyl ring carbon that is meta with respect to the phenyl ring carbon that is attached to the sulfur atom of the sulfonyl group.

In some embodiments, R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}, in which R^{d} can be as defined anywhere herein.

In certain embodiments, R³ is heteroaryl containing from 5-6 ring atoms, wherein from 1-4 of the ring atoms is independently selected from N, NH, N(C₁-C₆ alkyl), O, and S; and wherein said heteroaryl ring is optionally substituted with from 1-3 independently selected R^{d}, in which R^{d} can be as defined anywhere herein.

In certain embodiments, R³ is heteroaryl containing from 5-6 ring atoms, wherein from 1-4 of the ring atoms is independently selected from N, NH, N(C₁-C₆ alkyl), O, and S; and wherein said heteroaryl ring is substituted with from 1-3 (e.g., 1 or 2, e.g., 1) independently selected R^{d}, in which R^{d} can be as defined anywhere herein. For example, R³ can be optionally substituted thienyl, e.g., 5-chlorothienyl.

Non-Limiting Combinations of R′, A, R² and R³

[I-A]

In some embodiments:
W², W³, W⁵, and W⁶ are defined according to definition (A) as defined anywhere herein; and
R⁴ is selected from any of the substituents delineated in (i)-(iii) immediately below:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
   (ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
   (iii) heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
or
R⁴ is selected from:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
   (iii) heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a}; and
A is CH₂; and
R⁵ and R⁶ are defined according to (C); and R⁵ and R⁶, together with the carbon atom to which each is attached, is C₃-C₈ (e.g., C₃-C₆, e.g., C₅-C₆) cycloalkyl, which is optionally substituted with from 1-5 (e.g., 1-4-1-3, 1-2, 1)
R^{c}; and
R³ is C₆-C₁₀ aryl, which is optionally substituted with from 1-3 independently selected R^{d}.

[I-B]

In some embodiments, W², W³, W⁵, W⁶, R⁴, A, R⁵, and R⁶ can be as defined in [I-A], and R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}.

[I-C]

In some embodiments, each of W², W³, W⁵, and W⁶ is independently CH or C(halo); and R⁴, A, R³, R⁵, and R⁶ are each independently as defined in [I-A] or [I-B].

[I-D]

In some embodiments, one of W³ and W⁵ is CR′, and the other of W³ and W⁵ is CH or C(Halo); and each of W² and W⁶ is independently CH or C(halo); and A, R³, R⁵, and R⁶ are each independently as defined in [I-A] through [I-C]; and R⁴ is, e.g., H or C₁-C₆ alkoxy (e.g., OCH₃).

[I-E]

W², W³, W⁵, and W⁶ are defined according to definition (B) as defined anywhere herein; and R⁴, A, R³, R⁵, and R⁶ are each independently as defined in [I-A] or [I-D].

[I-F]

In some embodiments:
each of W², W³, W⁵, and W⁶ is CH;
R⁴ is —CO₂H; —C(O)OR⁴¹; —C(O)N(R⁴²)(R⁴³); —SO₂(R⁴⁵), or heterocyclyloxy;
A is CH₂;
R³, R⁵, and R⁶ are each independently as defined in [I-A] or [I-D].

Embodiments [I-A] through [I-F] can further include any one or more of the features described herein.

Compound Forms and Salts

In some embodiments, the compounds described herein may contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms may be present in a substituent, such as an alkyl group. All such isomeric forms, as well as mixtures thereof, of these compounds are expressly included in the present invention. The compounds described herein may also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present invention. The compounds of this invention may also be represented in multiple tautomeric forms; in such instances, the invention expressly includes all tautomeric forms of the compounds described herein, even though only a single tautomeric form may be represented. All such isomeric forms of such compounds are expressly included in the present invention. Unless otherwise mentioned or indicated, the chemical designation of a compound encompasses the mixture of all possible stereochemically isomeric forms of that compound.

In certain embodiments, the present invention relates to a compound represented by any of the structures outlined herein, wherein the compound is a single stereoisomer. In embodiments, a particular stereoisomer can be substantially free of (e.g., contains less than about 5% of, less than about 2% of, less than about 1%, less than about 0.5% of) another isomer, e.g., its opposing enantiomer and/or one or more other diastereomers.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

In embodiments, the compounds described herein may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of this invention include those derived from pharmaceutically acceptable inorganic and organic acids and bases. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient.

Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

Lists of suitable salts are found in Remington's Pharmaceutical Sciences, 17th ed., Mack Publishing Company, Easton, Pa., 1985, p. 1418; Journal of Pharmaceutical Science, 66, 2 (1977); “Pharmaceutical Salts: Properties, Selection, and Use A Handbook; Wermuth, C. G. and Stahl, P. H. (eds.) Verlag Helvetica Chimica Acta, Zurich, 2002 [ISBN 3-906390-26-8]; and Berge et al. (1977) “Pharmaceutical Salts”, J. Pharm. Sci. 66:1-19; each of which is incorporated herein by reference in its entirety.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides compounds which are in a prodrug form. Prodrugs of the compounds described herein are those compounds that undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the invention which is administered as an ester (the “prodrug”), but then is metabolically hydrolyzed to the carboxylic acid, the active entity. In embodiments, the ester can be an alkyl ester (e.g., C₁-C₃ alkyl, e.g., CH₃ or CH₂CH₃; or C₃-C₆ alkyl, e.g., C₃-C₆ branched alkyl, e.g., t-butyl, isopropyl, isobutyl). Additional examples include peptidyl derivatives of a compound of the invention.

The invention also includes various hydrate and solvate forms of the compounds described herein.

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

Synthesis of Compounds of Formula (I)

The compounds described herein can be conveniently prepared in accordance with the procedures outlined in the Examples section, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in R. C. Larock, Comprehensive Organic Transformations, 2d.ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The processes described herein can be monitored according to any suitable method known in the art. For example, product formation can be monitored by spectroscopic means, such as nuclear magnetic resonance spectroscopy (e.g., ¹H or ¹³C), infrared spectroscopy (FT-IR), spectrophotometry (e.g., UV-visible), or mass spectrometry (MS), or by chromatography such as high performance liquid chromatography (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of solvents. Depending on the particular reaction step, suitable solvents for a particular reaction step can be selected.

The compounds of the invention can be prepared, for example, using the reaction pathways and techniques as described in the Examples section.

Pharmaceutical Compositions, Administration, and Use

The term “pharmaceutically acceptable carrier” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

In embodiments, the pharmaceutical compositions described herein may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

In embodiments, pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In some embodiments, the compounds described herein may contain one or more acidic functional groups and, thus, are capable of forming pharmaceutically-acceptable salts with pharmaceutically-acceptable bases. These salts can be prepared, e.g., in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: water soluble antioxidants, such as ascorbic acid, cysteine hydrochloride, sodium bisulfate, sodium metabisulf[iota]te, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. In embodiments, formulations of the compounds described herein (and salts thereof) include those suitable for oral, nasal, topical (including buccal and sublingual), rectal, vaginal and/or parenteral administration. The formulations may conveniently be presented in unit dosage form and may be prepared by any conventional methods known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%. In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention. Methods of preparing these formulations or compositions include the step of bringing into association a compound of the present invention with the carrier and, optionally, one or more accessory ingredients. In general the formulations are prepared by uniformly and intimately bringing into association a compound of the present invention with liquid carriers, or finely divided solid carriers, or both, and then, if necessary, shaping the product. Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients. Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations. When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier. The preparations of the present invention may be given orally, parenterally, topically, or rectally. They are of course given in forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral administrations are preferred. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually. Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts. A physician having ordinary skill in the art can readily determine and prescribe the effective amount of the pharmaceutical composition required. For example, the physician could start doses of the compounds of the invention employed in the pharmaceutical composition at levels lower than that required to achieve the desired therapeutic effect and then gradually increasing the dosage until the desired effect is achieved. In some embodiments, a compound or pharmaceutical composition of the invention is chronically provided to a subject with neurodegenerative disorders. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject with neurodegenerative disorders. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally, doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). However, lower or higher doses can be used. In some embodiments, the dose administered to a subject may be modified as the physiology of the subject changes due to age, disease progression, weight, or other factors. If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). In other embodiments, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds according to the invention may be formulated for administration in any convenient way for use in human or veterinary medicine, by analogy with other pharmaceuticals. According to the invention, compounds for treating neurological conditions or diseases can be formulated or administered using methods that help the compounds cross the blood-brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood. The unique morphologic characteristics of the brain capillaries that make up the BBB are: (a) epithelial-like high resistance tight junctions that literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, γ-secretase inhibitor compounds that cross the BBB are particularly useful for treating subjects with neurodegenerative disorders. In one embodiment, it is expected that γ-secretase inhibitors that are non-charged (e.g., not positively charged) and/or non-lipophilic may cross the BBB with higher efficiency than charged (e.g., positively charged) and/or lipophilic compounds. Therefore it will be appreciated by a person of ordinary skill in the art that some of the compounds of the invention might readily cross the BBB. Alternatively, the compounds of the invention can be modified, for example, by the addition of various substituents that would make them less hydrophilic and allow them to more readily cross the BBB. Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference). Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intraarterial infusion of hypertonic substances which transiently open the blood-brain barrier to allow passage of hydrophilic drugs. However, hypertonic substances are potentially toxic and may damage the blood-brain barrier.

Peptide compositions of the invention may be administered using chimeric peptides wherein the hydrophilic peptide drug is conjugated to a transportable peptide, capable of crossing the blood-brain barrier by transcytosis at a much higher rate than the hydrophilic peptides alone. Suitable transportable peptides include, but are not limited to, histone, insulin, transferrin, insulin-like growth factor I (IGF-I), insulin-like growth factor II (IGF-II), basic albumin and prolactin.

Antibodies are another method for delivery of compositions of the invention. For example, an antibody that is reactive with a transferrin receptor present on a brain capillary endothelial cell can be conjugated to a neuropharmaceutical agent to produce an antibody-neuropharmaceutical agent conjugate (U.S. Pat. No. 5,004,697 incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point between 8.0 to 11.0 (U.S. Pat. No. 5,527,527, incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596 incorporated herein in its entirety by reference), or polypeptides called receptor mediated permeabilizers (RMP) (U.S. Pat. No. 5,268,164 incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g. between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g. bradykinin) can be administered before or at the same time as an intravenous injection of a drug. Thus, the term “co administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, compounds of the invention can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the γ-secretase inhibitor (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836 and 6,407,137, the disclosures of which are incorporated herein by reference in their entirety.

The administration of the agents of the present invention may be for either prophylactic or therapeutic purpose. When provided prophylactically, the agent is provided in advance of disease symptoms such as any Alzheimer's disease symptoms. The prophylactic administration of the agent serves to prevent or reduce the rate of onset of symptoms. When provided therapeutically, the agent is provided at (or shortly after) the onset of the appearance of symptoms of actual disease. In some embodiments, the therapeutic administration of the agent serves to reduce the severity and duration of Alzheimer's disease.

### EXAMPLES

The invention will be further described in the following examples. It should be understood that these examples are for illustrative purposes only and are not to be construed as limiting this invention in any manner.

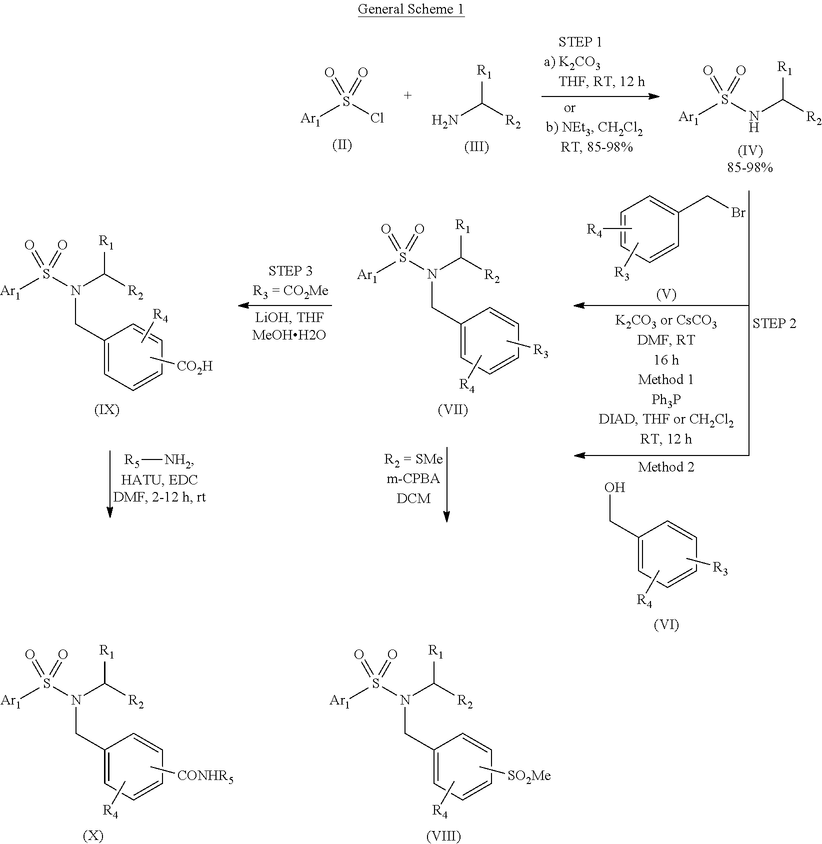

General Method A: Coupling of aryl sulfonyl chlorides with amines (Step 1, Scheme 1).

A solution of selected amine (III, 10.5 mmol) and triethyl amine (30 mmol) in anhydrous dichloromethane was added to a substituted phenylsulfonylchloride (II, 10 mmol). The reaction mixture was stirred for 16 h. A solution of 2N HCl was added with vigorous stirring to remove excessive triethyl amine. The organic layer was separated and washed with water, 10% sodium bicarbonate solution, water and brine, and then dried over sodium sulfate. Filtration and concentration in vacuo provided the sulfonamide product.

General Method B: Alkylation of Sulfonamides with Benzyl Halides (Step 2, Method 1, Scheme 1).

A solution of sulfonamide IV (1 mmol) and benzyl halide V (1.05 mmol) in anhydrous DMF (2-5 mL) was stirred with cesium cabonate (2 mmol) at room temperature for 2-12 h. Water (12-30 mL) was then added to the reaction mixture and extracted with ethyl acetate. The organic layer was separated and washed with water, brine and then dried. Filtration and concentration provided the crude coupled product which was purified by silica gel flash chromatography to yield the desired product VII.

General Method C: Alkylation of Sulfonamides with Benzyl Alcohols (Step 2, Method 2, Scheme 1).

To a solution of sulfonamide IV (1 mmol) in 6 mL of THF, Ph₃P (2.2 mmol) and the corresponding benzyl alcohol VI (1.5-2 mmol) were added, followed by DIAD (2.2 mmol). The reaction mixture was stirred at room temperature for 16 h. THF was removed in vacuo and the crude residue was purified by flash chromatography using 10-50% ethyl acetate/hexane to yield the alkylated sulfonamide compound VII.

General Method D: Hydrolysis of Benzoate Derivatives to Corresponding Acid Analogs (Step 3, Scheme 1).

A solution of benzoate VII (0.5 mmol) in 5 mL of THF was mixed with the solution of lithium hydroxide monohydrate (2 mmol) in 1 mL of water. The mixture was stirred at room temperature or heated in 60° C. for 4-5 h. Upon completion of the reaction, THF was then removed in vacuo. The reaction mixture was then acidified with 2-4 N HCl. The precipitate was filtered, washed with water and hexane and vacuum dried to give the desired acid IX. If there was no precipitate, then ethyl acetate was added to extract the product in which the organic layer was separated, washed with water and brine and then dried over sodium sulfate. Filtration and concentration in vacuo provided a solid which was triturated with a small amount of diethyl ether to give the final acid product IX.

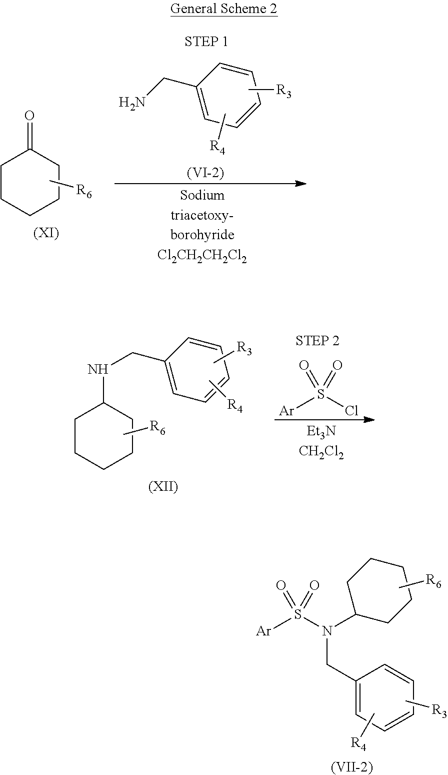

General Method E: Reductive Amination with Cyclohexanone (XI) and Amine (VI-2) (Step 1, Scheme 2)

A solution of a selected substituted cyclohexanones (XI, 17.22 mmol) and substituted benzyl primary amines (VI-2, 3.47 g, 17.22 mmol) in dichloroethane was stirred for 5 min. Sodium triacetoxyhydroborate (5.11 g, 24.11 mmol) was then added. The reaction mixture was stirred at room temperature for 16 h. Water was added to the reaction mixture and stirred for 5 min. The organic layer was then washed with NaHCO₃ and water, dried with sodium sulfate, filtered and concentrated in vacuo to give crude product which was purified by column chromatography using EtOAc/hexane (0 to 100% gradient) to yield the secondary amine product (XII).

General Method F: Coupling of Substituted Aryl Sulfonyl Chlorides with Secondary Amine (XII) (Step 2, Scheme 2)

To a solution of a selected substituted aryl sulfonyl chloride (0.550 g, 17.2.61 mmol) and secondary amine (XII, 2.61 mmol) in dichloroethane, triethylamine (0.725 ml, 5.21 mmol) was added. The reaction mixture was stirred at room temperature for 16 h and then washed with water, dried with sodium sulfate, and purified by column chromatography (silica gel) eluting with EtOAc/hexane (0-50%) to obtain various substituted sulfonamide products (VII-2). Sulfonamide (VII-2) corresponds to sulfonamide (VII) shown in Scheme 1, except the R₁ and R₂ of (VII) are now encumbered by a cycloalkyl ring system in sulfonamide (VII-2).

Standard synthetic methods are used to introduce various substituents on the rings of (VII) and (VII)-2), such as hydrolysis of alkyl esters to give carbolic acid analogs (IX). Coupling to this acid group with various substituted amines yielded our targeted amide analogs (X).

### Example 1 (AD965)

### 4-((4-Chloro-N-(tetrahydro-2H-pyran-4-yl)phenylsulfonamido)methyl)benzoic acid

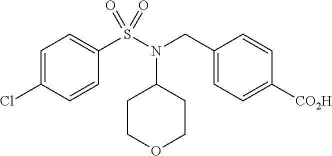 [SMILES: O=C(O)c1ccc(CN(C2CCOCC2)S(=O)(=O)c2ccc(Cl)cc2)cc1]

### Step 1

### 4-Chloro-N-(tetrahydro-2H-pyran-4-yl)benzenesulfonamide

A solution of 4-aminotetrahydropyran (2.36 g, 23.3 mmol) and triethyl amine (6.13 g, 60.6 mmol) in anhydrous dichloromethane was added to 4-chlorophenylsulfonylchloride (4.28 g, 20.2 mmol). The reaction mixture was stirred for 16 h to which 20 mL of 2N HCl was then added with vigorous stirring. The organic layer was separated and washed with water, 10% sodium bicarbonate solution, water and brine, and then dried over sodium sulfate. Filtration and concentration provided 5.11 g of an off-white solid which was triturated with diethyl ether to yield 4.80 g of a white solid.

MS (m/z): 275.3 (M⁺)

Mp 127-129° C.

### Step 2

### Methyl 4-((4-chloro-N-(tetrahydro-2H-pyran-4-yl)phenylsulfonamido)methyl)-benzoate

To a solution of 4-chloro-N-(tetrahydro-2H-pyran-4-yl)benzenesulfonamide (275 mg, 1 mmol), methyl 4-(hydroxymethyl)benzoate (352 mg, 2 mmol) and triphenyl phosphine (577 mg, 2.2 mmol) in anhydrous THF was added DIAD (445 mg, 2.2 mmol) dropwise. The reaction mixture was stirred at room temperature for 16 h. THF was then removed in vacuo to give a crude product that was purified by flash chromatography (hexane/ethyl acetate, 0-60%). A white solid product was isolated (189 mg, 44.6%).

MS (m/z): 423.3 (M⁺)

Mp 132-134° C.

### Step 3

To a solution of methyl 4-((4-chloro-N-(tetrahydro-2H-pyran-4-yl)phenyl-sulfonamido)-methyl)benzoate (120 mg, 0.283 mmol) in 4 mL THF was added 0.5 mL of water and methanol followed by lithium hydroxide hydrate (71.3 mg, 1.698 mmol). The reaction mixture was then heated for 5 h. After cooling to room temperature, 2N HCl was added. A white solid precipitated which was filtered and washed with water, ether, and hexane and then dried to give 106 mg of 4-((4-chloro-N-(tetrahydro-2H-pyran-4-yl)phenylsulfonamido)methyl)benzoic acid, the desired final product.

MS (m/z): 410.92 (M⁺+1)

Elemental Analysis: (C₁₉H₂₀ClNO₅S):

Calcd: C, 55.68%; H, 4.92%; N, 3.42%. Found: C, 55.87%; H, 5.01%; N, 3.51%.

Mp 218-219° C.

### Example 2 (AD1094)

### Methyl 4-((4-chloro-N-(2-methylcyclohexyl)phenylsulfonamido)methyl)benzoate

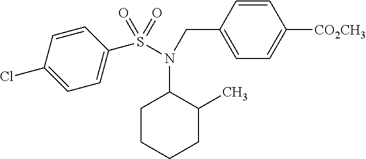 [SMILES: COC(=O)c1ccc(CN(C2CCCCC2C)S(=O)(=O)c2ccc(Cl)cc2)cc1]

### Step 1

### Chloro-N-(2-methylcyclohexyl)benzenesulfonamide

To a solution of potassium carbonate (589 mg, 4.26 mmol) and 2-methylcyclohexanamine (161 mg, 1.421 mmol) in THF (8 mL), 4-chlorobenzene-1-sulfonyl chloride (300 mg, 1.421 mmol) was added with stirring. The reaction mixture was stirred for 4 h and then diluted with ethyl acetate and water. The organic layer was separated and dried over MgSO₄. Recrystallization provided chloro-N-(2-methylcyclohexyl)benzenesulfonamide as a white solid in 54% yield.

### Step 2

To a solution of 4-chloro-N-(2-methylcyclohexyl)benzenesulfonamide (180 mg, 0.625 mmol) and methyl 4-(bromomethyl)benzoate (143 mg, 0.625 mmol) in DMF (3 mL), cesium carbonate (408 mg, 1.251 mmol) was added and the reaction mixture was stirred at room temperature for 4 h. Ethyl acetate and water were then added to the mixture, the layers were separated and the organic layer was dried over MgSO₄. Recrystallization gave methyl 4-((4-chloro-N-(2-methylcyclohexyl)phenylsulfonamido)methyl)benzoate as a while solid in 55% yield.

MS (m/z): 422.2

Mp 93-95° C.

### Example 3 (AD1112)

### 4-((4-Chloro-N-(2-methylcyclohexyl)phenylsulfonamido)methyl)benzoic acid

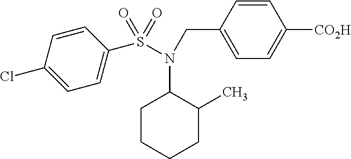 [SMILES: CC1CCCCC1N(Cc1ccc(C(=O)O)cc1)S(=O)(=O)c1ccc(Cl)cc1]

A solution of lithium hydroxide, H₂O (0.303 g, 7.23 mmol) and methyl 4-((4-chloro-N-(2-methylcyclohexyl)phenylsulfonamido)methyl)benzoate (1.05 g, 2.408 mmol) in THF (8 mL) and water (4 mL) was stirred at 50° C. for 24 h. The mixture was then concentrated in vacuo and diluted with water until a clear solution resulted. Upon acidification of the solution with 6N HCl, the desired product precipitated out which was filtered, washed with water and hexane and dried.

MS (m/z): 420.0

Elemental Analysis: C₂₁H₂₄ ClNO₄S:

Calcd: C, 59.78%, H, 5.73%, N, 3.32%. Found C, 59.51%, H, 5.71%, N, 3.17%.

Mp180-182° C.

### Example 4 (AD1113)

### 4-((4-Chloro-N-(2-methylcyclohexyl)phenylsulfonamido)methyl)-N-((R)-1-hydroxypropan-2-yl)benzamide

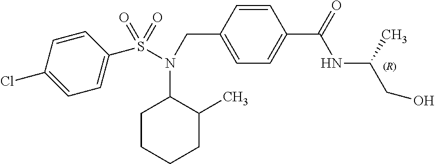

To the solution of 4-((4-chloro-N-(2-methylcyclohexyl)phenylsulfonamido)methyl)benzoic acid (200 mg, 0.474 mmol) and triethylamine (144 mg, 1.422 mmol) was added methanesulfonyl chloride (54 mg, 0.474 mmol) at −10° C. The mixture was stirred for 45 min and then (R)-2-aminopropan-1-ol (35.6 mg, 0.474 mmol) was added. The mixture was then stirred at room temperature for 16 h. After diluting the mixture with ethyl acetate and water, the organic layer was separated and concentrated in vacuo to give crude product. Purification by flash chromatography gave the desired product in 45% yield.

Elemental Analysis: C₂₄H₃₁ ClN₂O₄S:

Calcd: C, 60.17%, H, 6.52%, N, 5.85%. Found C, 60.42%, H, 6.74%, N5.69%.

### Example 5 (AD1138)

### 4-((4-Chloro-N-(2-methylcyclohexyl)phenylsulfonamido)methyl)-N—((S)-1-hydroxypropan-2-yl)benzamide

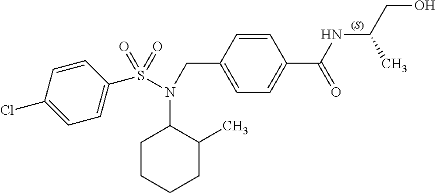

The synthetic procedure for Example 5 can be found in Example 4.

MS (m/z): 479.3

Mp 95-97° C.

### Example 6 (AD1139)

### 4-((4-Chloro-N-(2-methylcyclohexyl)phenylsulfonamido)methyl)-N-(2-hydroxyethyl)benzamide

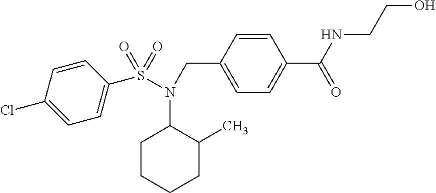 [SMILES: CC1CCCCC1N(Cc1ccc(C(=O)NCCO)cc1)S(=O)(=O)c1ccc(Cl)cc1]

The synthetic procedure for Example 6 can be found in Example 4.

MS (m/z): 465.4

Mp 105-107° C.

### Example 7 (AD1143)

### Methyl 5-((4-chloro-N-(tetrahydro-2H-pyran-4-yl)phenylsulfonamido)methyl)-2-methoxybenzoate

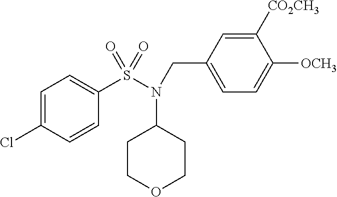 [SMILES: COC(=O)c1cc(CN(C2CCOCC2)S(=O)(=O)c2ccc(Cl)cc2)ccc1OC]

A mixture of 4-chloro-N-(tetrahydro-2H-pyran-4-yl)benzenesulfonamide (300 mg, 1.088 mmol), methyl 5-(bromomethyl)-2-methoxybenzoate (296 mg, 1.142 mmol) and cesium carbonate in 2 mL DMF was stirred at room temperature for 3 h. Water (28 mL) was then added to the reaction mixture followed by extraction with ethyl acetate. The organic layer was separated and washed with water, brine and dried. Filtration and concentration in vacuo of the organic layer yielded 533 mg of an oil which was purified by flash chromatography (hexane/ethyl acetate 0-40%) to give 316 mg of final product.

MS: (m/z) 454.06 (M⁺+1)

Elemental Analysis: (C₂₁H₂₄ClNO₆S):

Calcd: C, 55.56%; H, 5.33%; N, 3.09%. Found: C, 55.53%; H, 5.19%; N, 3.04%.

### Example 8 (AD1144)

### 5-((4-Chloro-N-(tetrahydro-2H-pyran-4-yl)phenylsulfonamido)methyl)-2-methoxy-benzoic acid

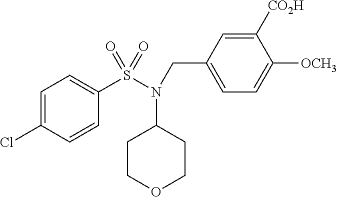 [SMILES: COc1ccc(CN(C2CCOCC2)S(=O)(=O)c2ccc(Cl)cc2)cc1C(=O)O]

To a solution of methyl 5-((4-chloro-N-(tetrahydro-2H-pyran-4-yl)phenylsulfonamido)-methyl)-2-methoxybenzoate (225 mg, 0.496 mmol) in 4 mL of THF was added an aqueous solution of LiOH (1 mL) The mixture was stirred at 60° C. for 16 h. THF was then removed in vacuo to give a residue to which water (1 mL) and 4 N HCl were added until pH 2 was reached. A white solid precipitated which was then filtered and washed with water and hexane and dried to yield 205 mg (94%) of the desired product.

MS (m/z): 440.06 (M⁺+1)

Elemental Analysis: (C₂₀H₂₂ClNO₆S):

Calcd: C, 54.61%; H, 5.04%; N, 3.18%. Found: C, 54.21%; H, 4.81%; N, 3.11%

Mp 174-176° C.

### Example 9 (AD1145)

### 4-((N-cyclohexyl-4-fluorophenylsulfonamido)methyl)benzoic acid

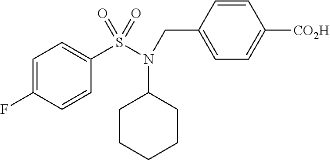 [SMILES: O=C(O)c1ccc(CN(C2CCCCC2)S(=O)(=O)c2ccc(F)cc2)cc1]

### Step 1

### N-cyclohexyl-4-fluorobenzenesulfonamide

The title compound (3.48 g, 87.7%) was prepared from 4-fluorophenylsulfonyl chloride (3.0 g, 15.4 mmol) and cyclohexyl amine (1.68 g, 17 mmol) according to the General Method A.

MS (m/z): 258.1 (M⁺+1).

### Step 2

### Methyl 4-((N-cyclohexyl-4-fluorophenylsulfonamido)methyl)benzoate

The title compound (632 mg, 78%) was prepared from N-cyclohexyl-4-fluorobenzenesulfonamide (0.515 g, 2 mmol) and methyl 4-(bromomethyl)benzoate (0.481 g, 2.100 mmol) according to the General Method B.

MS (m/z): 406.1 (M⁺+1).

### Step 3

### 4-((N-cyclohexyl-4-fluorophenylsulfonamido)methyl)benzoic acid

Methyl 4-((N-cyclohexyl-4-fluorophenylsulfonamido)methyl)benzoate (200 mg, 0.49 mmol) was hydrolyzed according to the General Method D to yield 4-((N-cyclohexyl-4-fluorophenylsulfonamido)methyl)benzoic acid, the desired product (145 mg, 75%).

MS (m/z): 392.10 (M⁺+1)

Elemental Analysis: (C₂₀H₂₂FNO₄S):

Calcd: C, 61.36%; H, 5.66%; N, 3.58%. Found: C, 61.30%; H, 5.46%; N, 3.51%

Mp 218-220° C.

### Example 10 (AD953)

### 4-Chloro-N-(4-(methylthio)benzyl)-N-(tetrahydro-2H-pyran-4-yl)benzene-sulfonamide

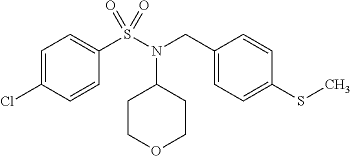 [SMILES: CSc1ccc(CN(C2CCOCC2)S(=O)(=O)c2ccc(Cl)cc2)cc1]

The title compound (179 mg, 30%) was prepared from 4-chloro-N-(tetrahydro-2H-pyran-4-yl)benzenesulfonamide (275 mg, 1 mmol) and (4-(methylthio)phenyl)methanol (308 mg, 2 mmol) according to the General Method C.

MS (m/z): 412.10 (M⁺+1)

Mp 124-127° C.

### Example 11 (AD954)

### 4-Chloro-N-(4-(methylsulfonyl)benzyl)-N-(tetrahydro-2H-pyran-4-yl)benzene-sulfonamide

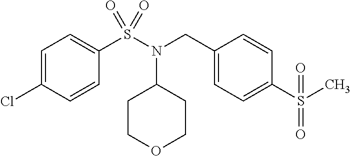 [SMILES: CS(=O)(=O)c1ccc(CN(C2CCOCC2)S(=O)(=O)c2ccc(Cl)cc2)cc1]

To a solution of 4-chloro-N-(4-(methylthio)benzyl)-N-(tetrahydro-2H-pyran-4-yl)benzenesulfonamide (135 mg, 0.328 mmol) in methylene dichloride (5 mL) was added m-chloroperoxybenzoic acid (220 mg, 0.983 mmol). The reaction mixture was stirred at room temperature and monitored by TLC(CH₂Cl₂/EtOAc=10:1) until complete. After the mixture was stirred for 30 min, the starting material was mostly consumed. After an additional 6 h of stirring at room temperature, the mixture was then quenched by adding DMSO (70 μL) and then stirred for 15 minutes. The pH of the reaction mixture was then brought to pH 11 using aqueous Na₂CO₃ solution. The organic layer was then separated and concentrated in vacuo followed by extraction with ethyl acetate. The organic layer was then washed with water and brine and dried over sodium sulfate and filtered. Concentration of the organic layer yielded 141 mg of crude white product which was purified by flash chromatography (dichloromethane/ethyl acetate, 0-40%) to give 4-chloro-N-(4-(methylsulfonyl)benzyl)-N-(tetrahydro-2H-pyran-4-yl)benzene-sulfonamide as a white solid (122 mg, 84%).

MS (m/z): 443.1 (M⁺)

Mp 158-160° C.

### Example 12 (AD955)

### Methyl 4-((4-chloro-N-(tetrahydro-2H-pyran-4-yl)phenylsulfonamido)methyl)-benzoate

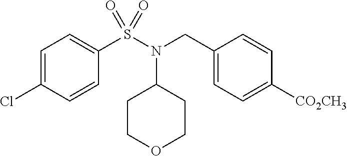 [SMILES: COC(=O)c1ccc(CN(C2CCOCC2)S(=O)(=O)c2ccc(Cl)cc2)cc1]

The title compound is described in Example 1, Step 2.

### Example 13 (AD956)

### 4-Chloro-N-(tetrahydro-2H-pyran-4-yl)-N-((6-(tetrahydro-2H-pyran-4-yloxy)-pyridin-3-yl)methyl)benzenesulfonamide

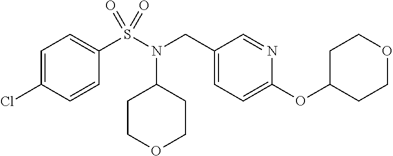 [SMILES: O=S(=O)(c1ccc(Cl)cc1)N(Cc1ccc(OC2CCOCC2)nc1)C1CCOCC1]

The title compound (237 mg, 51%) was prepared from 4-chloro-N-(tetrahydro-2H-pyran-4-yl)-benzenesulfonamide (138 mg, 0.5 mmol) and (6-(tetrahydro-2H-pyran-4-yloxy)pyridin-3-yl)methanol (209 mg, 1 mmol) according to the General Method C.

MS (m/z): 467.4 (M⁺+1)

Mp: 123-125° C.

### Example 14 (AD1136)

### 4-((4-Chloro-N-(2-methylcyclohexyl)phenylsulfonamido)methyl)-N-((R)-1-methoxypropan-2-yl)benzamide

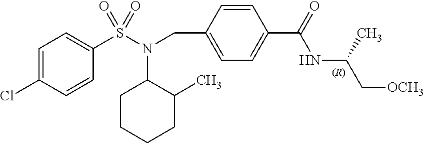

Sodium hydride (15 mg, 0.366 mol, 60% in mineral oil) was added to a solution of 4-((4-chloro-N-(2-methylcyclohexyl)phenylsulfonamido)methyl)-N-((R)-1-hydroxypropan-2-yl)benzamide (167 mg, 0.349 mmol) in anhydrous THF (1.8 mL) at −60 C. The reaction mixture was stirred for 1 h and iodomethane (0.366 mmol) was added at −60° C. The mixture was gradually warmed up to room temperature over 5 h. After a standard work-up procedure, the crude product was purified using flash chromatography to give 23 mg (13%) of final product.

### Example 15 (AD1155)

### (S)-4-((4-Chloro-N-(1-cyclohexylpropyl)phenylsulfonamido)methyl)benzoic acid

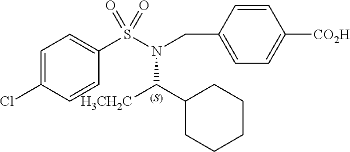

Example 131 was prepared via the General Method described in Scheme 1.

MS (m/z): 448.4

Mp 147-149° C.

### Example 16 (AD1159)

### Methyl 5-((4-chloro-N-((tetrahydro-2H-pyran-4-yl)methyl)phenylsulfonamido)-methyl)-2-methoxybenzoate

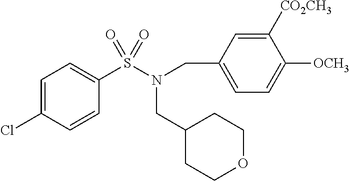 [SMILES: COC(=O)c1cc(CN(CC2CCOCC2)S(=O)(=O)c2ccc(Cl)cc2)ccc1OC]

### Step 1

### 4-Chloro-N-((tetrahydro-2H-pyran-4-yl)methyl)benzenesulfonamide

A mixture of (tetrahydro-2H-pyran-4-yl)methanamine hydrochloric salt (1 g, 6.59 mmol), 4-chlorobenzene-1-sulfonyl chloride (1.326 g, 6.28 mmol) and potassium carbonate (2.170 g, 15.70 mmol) in 10 mL of THF was stirred at room temperature for 16 h. Water was added (15 mL) and THF was then removed in vacuo to afford a residue that was extracted with ethyl acetate. The organic layer was separated and washed with water, brine and dried. After standard workup, 1.45 g of white solid was collected as crude product which was then triturated three times with diethyl ether to yield a white solid (1.35 g). The solid was further triturated with diethyl ether four times, and 1.31 g (72.3%) of product was collected.

MS (m/z): 289.20 (M⁺+1)

### Step 2

### Methyl 5-((4-chloro-N-((tetrahydro-2H-pyran-4-yl)methyl)phenylsulfonamido)-methyl)-2-methoxybenzoate

To a mixture of 4-chloro-N-((tetrahydro-2H-pyran-4-yl)methyl)benzenesulfonamide (290 mg, 1.0 mmol) and methyl 5-(bromomethyl)-2-methoxybenzoate (285 mg, 1.10 mmol) in DMF (3 mL) was added cesium carbonate (652 mg, 2.0 mmol). The reaction mixture was stirred at room temperature for 2 h and then water (12 mL) was added. The mixture was extracted with ethyl acetate and the organic layer was separated and washed with water, brine and dried. Filtration and concentration yielded 459 mg of crude product which was purified by flash chromatography (hexane/ethyl acetate, 0-40%) to give 315 mg (67%) of 4-chloro-N-((tetrahydro-2H-pyran-4-yl)methyl)benzenesulfonamide as a white crystalline solid.

MS (m/z): 468.09 (M⁺+1)

Elemental Analysis: C₂₂H₂₆ClNO₆S):

Calcd: C, 56.47%; H, 5.6%; N, 2.99%. Found: C, 56.62%; H, 5.32%; N, 3.06%

Mp 118-119° C.

### Example 17 (AD1161)

### 5-((4-Chloro-N-((tetrahydro-2H-pyran-4-yl)methyl)phenylsulfonamido)methyl)-2-methoxybenzoic acid

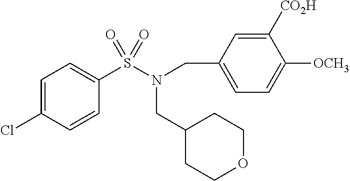 [SMILES: COc1ccc(CN(CC2CCOCC2)S(=O)(=O)c2ccc(Cl)cc2)cc1C(=O)O]

A solution of methyl 5-((4-chloro-N-((tetrahydro-2H-pyran-4-yl)methyl)phenyl-sulfonamido)methyl)-2-methoxybenzoate (171.5 mg, 0.366 mmol) in THF was added to a solution of lithium hydroxide hydrate (61.5 mg, 1.466 mmol) in water (1 mL) The reaction mixture was stirred and heated in a sealed pressure tube for 5 h. The reaction was then cooled to room temperature, THF was removed in vacuo and then water (1 mL) was added. 4 N HCl was added dropwise to acidify the mixture to pH 2 and then the mixture was extracted with ethyl acetate. The organic layer was separated and washed with water, brine and dried. Filtration and concentration in vacuo provided 145 mg (87%) of 5-((4-chloro-N-((tetrahydro-2H-pyran-4-yl)methyl)phenylsulfonamido)methyl)-2-methoxybenzoic acid as a white solid.

MS (m/z): 454.08 (M⁺+1)

Elemental Analysis: C₂₁H₂₄ ClNO₆S:

Calcd: C, 55.56%; H, 5.33%; N, 3.09%. Found: C, 55.36%; H, 5.42%; N, 3.06%

Mp 158-159° C.

### Example 18 (AD1162)

### Methyl 4-((4-chloro-N-((tetrahydro-2H-pyran-4-yl)methyl)phenylsulfonamido)-methyl)benzoate

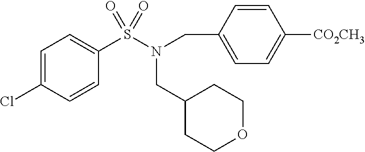 [SMILES: COC(=O)c1ccc(CN(CC2CCOCC2)S(=O)(=O)c2ccc(Cl)cc2)cc1]

A mixture of 4-chloro-N-((tetrahydro-2H-pyran-4-yl)methyl)benzenesulfonamide (300 mg, 1.035 mmol), methyl 4-(bromomethyl)benzoate (261 mg, 1.139 mmol) and cesium carbonate (675 mg, 2.071 mmol) in DMF (3 mL) was stirred at room temperature for 3 h. Water (12 mL) was added and the mixture was extracted with ethyl acetate. The organic layer was separated and washed with water, brine and dried. Filtration and concentration in vacuo yielded 447 mg of crude product which was purified by flash chromatography (hexane/ethyl acetate, 0-50%) to give 326 mg (72%) of methyl 4-((4-chloro-N-((tetrahydro-2H-pyran-4-yl)methyl)phenylsulfonamido)-methyl)benzoate as a white crystalline solid.

MS (m/z): 438.10 (M⁺+1)

Elemental Analysis: C₂₁H₂₄ClNO₅S:

Calcd: C, 57.59%; H, 5.52%; N, 3.20%. Found: C, 57.73%; H, 5.52%; N, 3.25%

Mp 115-117° C.

### Example 19 (AD1163)

### 4-((4-Chloro-N-((tetrahydro-2H-pyran-4-yl)methyl)phenylsulfonamido)methyl)-benzoic acid

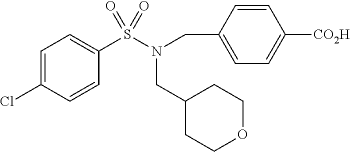 [SMILES: O=C(O)c1ccc(CN(CC2CCOCC2)S(=O)(=O)c2ccc(Cl)cc2)cc1]

A solution of methyl 4-((4-chloro-N-((tetrahydro-2H-pyran-4-yl)methyl)phenyl-sulfonamido)methyl)benzoate (200 mg, 0.457 mmol) in THF (5 mL) was added to a solution of lithium hydroxide hydrate (77 mg, 1.827 mmol) in water (1 mL) The mixture was heated at 55° C. for 6 h and then cooled to room temperature. 4 N HCl was added dropwise to bring the mixture to pH 2. THF was then removed in vacuo and a white solid precipitated out, was filtered and then washed with water and hexane and dried to give 4-((4-chloro-N-((tetrahydro-2H-pyran-4-yl)methyl)phenylsulfonamido)methyl)-benzoic acid, the desired product (190 mg, 98%).

MS (m/z): 424.07 (M⁺+1)

Elemental Analysis: C₂₀H₂₂ClNO₅S:

Calcd: C, 56.67%; H, 5.23%; N, 3.3%. Found: C, 56.19%; H, 5.23%; N, 3.27%

Mp 188-190° C.

### Example 20 (AD1164)

### (S)-4-((4-Chloro-N-(1-cyclopentylpropyl)phenylsulfonamido)methyl)benzoic acid

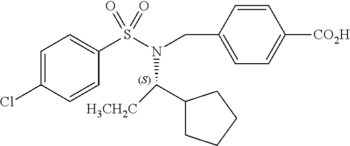

Example 20 was prepared via the General Method described in Scheme 1.

MS (m/z): 434.1

Elemental Analysis: C₂₂H₂₆ClNO₄S:

Calcd: C, 60.61%, H, 6.01%, N, 3.21%. Found 60.31%, 5.97%, 3.23%

Mp 67-69° C.

### Example 21 (AD1165)

### (S)-Methyl 4-((4-chloro-N-(1-cyclopentylpropyl)phenylsulfonamido)methyl)benzoate

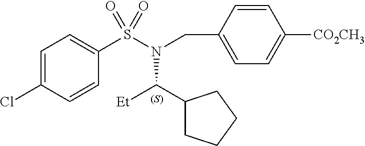

Example 21 was prepared via the General Method described in Scheme 1.

MS (m/z): 448.3

Elemental Analysis: C₂₃H₂₈ClNO₄S:

Calcd: C, 61.39%, H, 6.27%, N, 3.11%. Found, 61.13%, H, 6.26%, N, 3.20%

### Example 22 (AD1166)

### (R)-Methyl 4-((4-chloro-N-(1-cyclopentylpropyl)phenylsulfonamido)methyl)benzoate

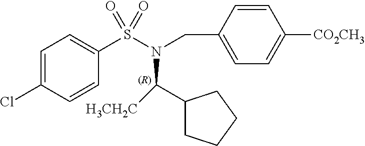

Example 22 was prepared via the General Method described in Scheme 1.

MS (m/z): 448.3

Mp 81-83° C.

### Example 23 (AD1167)

### (R)-4-((4-Chloro-N-(1-cyclopentylpropyl)phenylsulfonamido)methyl)benzoic acid

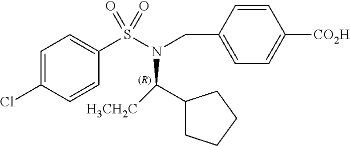

Example 23 was prepared via the General Method described in Scheme 1.

MS (M/Z): 434.1

Mp 67-69° C.

BWH 21392 Filing: Oct. 4, 2011-Oct. 4, 2012:

Substituted Cycloalkyl Sulfonamides as Gamma-Secretase Inhibitors or Modulators for the Treatment of Alzheimer's Disease

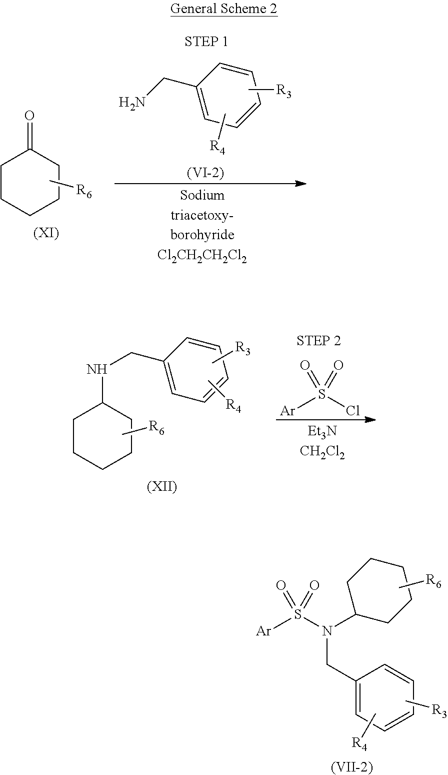

General Method E: Reductive Amination with Cyclohexanone (XI) and Amine (VI-2) (Step 1, Scheme 2)

A solution of a selected substituted cyclohexanones (XI, 17.22 mmol) and substituted benzyl primary amines (VI-2, 3.47 g, 17.22 mmol) in dichloroethane was stirred for 5 min. Sodium triacetoxyhydroborate (5.11 g, 24.11 mmol) was then added. The reaction mixture was stirred at room temperature for 16 h. Water was added to the reaction mixture and stirred for 5 min. The organic layer was then washed with NaHCO₃ and water, dried with sodium sulfate, filtered and concentrated in vacuo to give crude product which was purified by column chromatography using EtOAc/hexane (0 to 100% gradient) to yield the secondary amine product (XII).

General Method F: Coupling of Substituted Aryl Sulfonyl Chlorides with Secondary Amine (XII) (Step 2, Scheme 2)

To a solution of a selected substituted aryl sulfonyl chloride (0.550 g, 17.2.61 mmol) and secondary amine (XII, 2.61 mmol) in dichloroethane, triethylamine (0.725 ml, 5.21 mmol) was added. The reaction mixture was stirred at room temperature for 16 h and then washed with water, dried with sodium sulfate, and purified by column chromatography (silica gel) eluting with EtOAc/hexane (0-50%) to obtain various substituted sulfonamide products (VII-2). Sulfonamide (VII-2) corresponds to sulfonamide (VII) shown in Scheme 1, except the R₁ and R₂ of (VII) are now encumbered by a cycloalkyl ring system in sulfonamide (VII-2).

Standard synthetic methods are used to introduce various substituents on the rings of (VII) and (VII)-2), such as hydrolysis of alkyl esters to give carbolic acid analogs (IX). Coupling to this acid group with various substituted amines yielded our targeted amide analogs (X).

### Example 24 (AD1188)

### 4-((4-Chloro-N-(2-hydroxycyclohexyl)phenylsulfonamido)methyl)benzoic acid

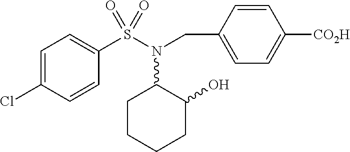 [SMILES: O=C(O)c1ccc(CN(C2CCCCC2O)S(=O)(=O)c2ccc(Cl)cc2)cc1]

To a solution of methyl 4-((4-chloro-N-(2-hydroxycyclohexyl)phenylsulfonamido) methyl)benzoate (290 mg, 0.662 mmol) in 5 mL of THF was added 1 mL of lithium hydroxide hydrate (111 mg, 2.65 mmol) aqueous solution. The mixture was stirred and heated at 50° C. for 16 h. TLC showed that the reaction was complete. 4 N HCl was then added dropwise to bring the reaction mixture to pH 2. THF was then removed in vacuo and a white solid precipitated. This solid was filtered, washed with water, hexane and 0.5 mL of diethyl ether and then dried in a vacuum oven for 16 h. 4-((4-Chloro-N-(2-hydroxycyclohexyl)phenylsulfonamido)methyl)benzoic acid was isolated in 99% yield (279 mg).

Mp 218-219° C.

¹H NMR (500 MHz, CD₃OD) δ 7.93 (d, J=6.5 Hz, 2H), δ 7.80 (m, 2H), δ 7.53 (d, J=6.5 Hz, 2H), δ 7.48 (m, 2H), δ 4.61 (d, J=13 Hz, 1H), δ 4.42 (d, J=13 Hz, 1H), δ 3.56 (m, 1H), δ 3.42 (m, 1H), δ 1.94 (m, 1H), δ 1.58 (m, 3H), δ 1.24 (m, 1H), δ 1.17 (m, 2H), δ 1.06 (m, 1H).

### Example 25 (AD1189)

### Methyl 4-((4-chloro-N-(2-hydroxycyclohexyl)phenylsulfonamido)methyl)benzoate

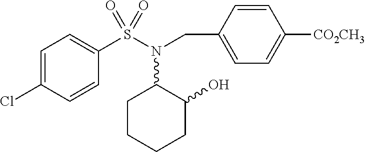 [SMILES: COC(=O)c1ccc(CN(C2CCCCC2O)S(=O)(=O)c2ccc(Cl)cc2)cc1]

A mixture of 4-chloro-N-(2-hydroxycyclohexyl)benzenesulfonamide (0.869 g, 3 mmol), methyl 4-(bromomethyl)benzoate (0.722 g, 3.15 mmol) and cesium carbonate (1.955 g, 6.00 mmol) in 5 mL of DMF was stirred at room temperature for 4 h. TLC showed that the reaction was complete. 40 mL of water was added to the reaction mixture and then the mixture was extracted with ethyl acetate (3×40 mL) The organic layer was washed with water, brine and dried. Filtration and concentration in vacuo yielded 1.237 g of white solid which was recrystallized in acetone to give methyl 4-((4-chloro-N-(2-hydroxycyclohexyl)phenylsulfonamido) methyl)benzoate, a white solid (1.10 g, 84% yield).

Mp 162-164° C.

¹H NMR (500 MHz, CDCl₃) δ 8.0 (d, J=5.5 Hz, 2H), δ 7.75 (m, 2H), δ 7.47 (m, 4H), δ 4.60 (d, J=13 Hz, 1H), δ 4.37 (d, J=13 Hz, 1H), δ 3.92 (s, 3H), δ 3.50 (m, 1H), δ 3.18 (m, 1H), δ 1.96 (m, 1H), δ 1.62 (m, 2H), δ 1.49 (m, 1H), δ 1.18 (m, 3H), δ 1.01 (m, 1H)

### Example 26 (AD1178)

### 4-Chloro-N-(4-cyano-2-fluorobenzyl)-N-cyclobutylbenzenesulfonamide

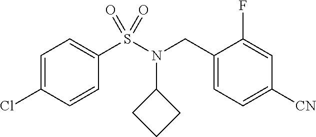 [SMILES: N#Cc1ccc(CN(C2CCC2)S(=O)(=O)c2ccc(Cl)cc2)c(F)c1]

To a stirred solution of 4-chloro-N-cyclobutylbenzenesulfonamide (0.491 g, 2 mmol) and 4-(bromomethyl)-3-fluorobenzonitrile (0.514 g, 2.400 mmol) in 6 mL of DMF was added K₂CO₃ (1.106 g, 8.0 mmol, 4.0 equiv) at room temperature. After stirring for 16 h, the reaction mixture was quenched with 20 mL of water and extracted with ethyl acetate (2×30 mL) The combined organic extracts were washed with a saturated aqueous Na₂CO₃ solution and brine, and then dried over Na₂SO₄. The mixture was concentrated in vacuo to give crude product. Purification by column chromatography using 20% ethyl acetate/hexane yielded 4-chloro-N-(4-cyanobenzyl)-N-(pentan-3-yl)benzenesulfonamide, a white solid (492 mg, 65%).

Elemental Analysis: (C₁₈H₁₆ClFN₂O₂S):

Calcd: C, 57.07%, H, 4.26%, N, 7.39%. Found C, 57.33%, H, 4.03%, N, 7.45%

Mp 125-127° C.

### Example 27 (AD1179)

### N-(4-Cyano-2-fluorobenzyl)-N-cyclobutyl-4-methylbenzenesulfonamide

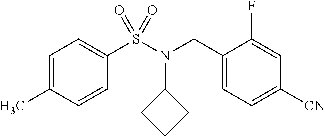 [SMILES: Cc1ccc(S(=O)(=O)N(Cc2ccc(C#N)cc2F)C2CCC2)cc1]

N-(4-Cyano-2-fluorobenzyl)-N-cyclobutyl-4-methylbenzenesulfonamide (444 mg, 62% yield) was prepared from N-cyclobutyl-4-methylbenzenesulfonamide and 4-(bromomethyl)-3-fluorobenzonitrile according to the procedure described for Example 26.

Elemental Analysis: (C₁₉H₁₉FN₂O₂S):

Calcd: C, 63.67%, H, 5.34%, N, 7.82%. Found C, 63.86%, H, 5.24%, N, 7.84%.

Mp 146-147° C.

### Example 28 (AD1180)

### 4-Chloro-N-(4-cyano-2-fluorobenzyl)-N-cyclopentylbenzenesulfonamide

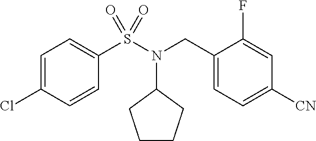 [SMILES: N#Cc1ccc(CN(C2CCCC2)S(=O)(=O)c2ccc(Cl)cc2)c(F)c1]

4-Chloro-N-(4-cyano-2-fluorobenzyl)-N-cyclopentylbenzenesulfonamide (597 mg, 76% yield) was prepared from 4-chloro-N-cyclopentylbenzenesulfonamide and 4-(bromomethyl)-3-fluorobenzonitrile according to the procedure described for Example 26.

Elemental Analysis: (C₁₉H₁₈ClFN₂O₂S):

Calcd: C, 58.09%, H, 4.62%, N, 7.13%. Found C, 57.96%, H, 4.53%, N, 7.10%

Mp 176-177° C.

### Example 29 (AD1181)

### N-(4-Cyano-2-fluorobenzyl)-N-cyclopentyl-4-methylbenzenesulfonamide

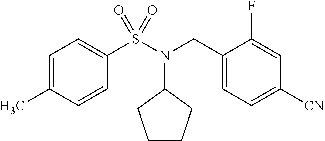 [SMILES: Cc1ccc(S(=O)(=O)N(Cc2ccc(C#N)cc2F)C2CCCC2)cc1]

N-(4-Cyano-2-fluorobenzyl)-N-cyclopentyl-4-methylbenzenesulfonamide (491 mg, 66% yield) was prepared from N-cyclopentyl-4-methylbenzenesulfonamide and 4-(bromomethyl)-3-fluorobenzonitrile according to the procedure described for Example 26.

Elemental Analysis: (C₂₀H₂₁FN₂O₂S):

Calcd: C, 64.49%, H, 5.68%, N, 7.52%. Found C, 64.36%, H, 5.74%, N, 7.48%

Mp 178-179° C.

### Example 30 (AD1192)

### 4-((4-Chloro-N-((1S,2S)-2-hydroxycyclohexyl)phenylsulfonamido)methyl)benzoic acid

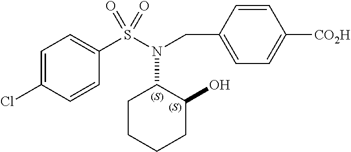

To a solution of methyl 4-((4-chloro-N-(2-hydroxycyclohexyl)phenylsulfonamido) methyl)benzoate (290 mg, 0.662 mmol) in 5 mL of THF was added 1 mL of lithium hydroxide hydrate (111 mg, 2.65 mmol) aqueous solution. The mixture was stirred and heated at 50° C. for 16 h. TLC showed that the reaction was complete. 4 N HCl was then added dropwise to bring the reaction mixture to pH 2. THF was then removed in vacuo and a white solid precipitated. This solid was filtered, washed with water, hexane and 0.5 mL of diethyl ether and then dried in a vacuum oven for 16 h to give 4-((4-chloro-N-((1S,2S)-2-hydroxycyclohexyl)phenylsulfonamido)methyl)benzoic acid, a white solid (279 mg, 95% yield).

Mp 218-219° C.

¹H NMR (500 MHz, CD₃OD) δ 7.93 (d, J=6.5 Hz, 2H), δ 7.80 (m, 2H), δ 7.53 (d, J=6.5 Hz, 2H), δ 7.48 (m, 2H), δ 4.61 (d, J=13 Hz, 1H), δ 4.42 (d, J=13 Hz, 1H), δ 3.56 (m, 1H), δ 3.42 (m, 1H), δ 1.94 (m, 1H), δ 1.58 (m, 3H), δ 1.24 (m, 1H), δ 1.17 (m, 2H), δ 1.06 (m, 1H)

### Example 31 (AD1193)

### Methyl 4-((4-chloro-N-((1S,2S)-2-hydroxycyclohexyl)phenylsulfonamido)methyl)benzoate

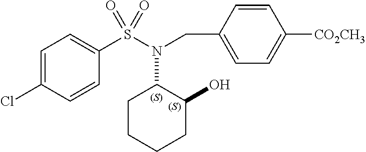

A mixture of 4-chloro-N-((1S,2S)-2-hydroxycyclohexyl)benzenesulfonamide (700 mg, 2.416 mmol), methyl 4-(bromomethyl)benzoate (581 mg, 2.54 mmol) and cesium carbonate (1574 mg, 4.83 mmol) in 2 mL of DMF was stirred at room temperature. After 3 hours, 16 mL of water was added to the reaction mixture. The mixture was then extracted with ethyl acetate (3×40 mL) and the combined extracts were washed with water, brine and dried over Na₂SO₄. Filtration and concentration in vacuo gave a crude product which was then purified by combiflash chromatography using 20% ethyl acetate/hexane to give methyl 4-((4-chloro-N-((1S,2S)-2-hydroxycyclohexyl)phenylsulfonamido)methyl)benzoate, a white solid (825 mg, 78% yield).

Mp 150-152° C.

¹H NMR (500 MHz, CDCl₃) δ 8.0 (d, J=5.5 Hz, 2H), δ 7.75 (m, 2H), δ 7.47 (m, 4H), δ 4.60 (d, J=13 Hz, 1H), δ 4.37 (d, J=13 Hz, 1H), δ 3.92 (s, 3H), δ 3.50 (m, 1H), δ 3.18 (m, 1H), δ 1.96 (m, 1H), δ 1.62 (m, 2H), δ 1.49 (m, 1H), δ 1.18 (m, 3H), δ 1.01 (m, 1H)

### Example 32 (AD1194)

### 4-((N-(2-Methylcyclohexyl)-6-(trifluoromethyl)pyridine-3-sulfonamido)methyl)benzoic acid

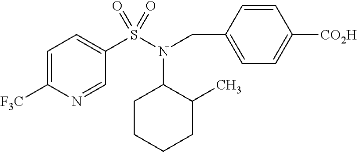 [SMILES: CC1CCCCC1N(Cc1ccc(C(=O)O)cc1)S(=O)(=O)c1ccc(C(F)(F)F)nc1]

### Step 1

### 4-Chloro-N-(4-cyanobenzyl)-N-(pentan-3-yl)benzenesulfonamide

To a stirred solution of N-(2-methylcyclohexyl)-6-(trifluoromethyl)pyridine-3-sulfonamide (600 mg, 1.86 mmol) and methyl 4-(bromomethyl)benzoate (853 mg, 3.72 mmol) in 6 mL of DMF was added K₂CO₃ (1.03 g, 7.45 mmol, 4.0 equiv) at room temperature. After stirring for 16 h, the reaction mixture was quenched with 20 mL of water and extracted with ethyl acetate (2×30 mL) The combined organic extracts were washed with a saturated aqueous Na₂CO₃ solution and brine, and then dried over Na₂SO₄. The mixture was concentrated in vacuo to give crude product. Purification by column chromatography using 20% ethyl acetate/hexane yielded 4-chloro-N-(4-cyanobenzyl)-N-(pentan-3-yl)benzenesulfonamide, a white solid (640 mg, 73%).

Mp 124-126° C.

### Step 2

### 4-((N-(2-Methylcyclohexyl)-6-(trifluoromethyl)pyridine-3-sulfonamido)methyl)benzoic acid

4-((N-(2-methylcyclohexyl)-6-(trifluoromethyl)pyridine-3-sulfonamido)methyl)benzoate (640 mg, 1.36 mmol) and lithium hydroxide hydrate (171 mg, 4.08 mmol) were stirred in THF (8 mL) and H₂O (4 mL) at room temperature for 16 h. THF was removed in vacuo and then 10 mL of water was added to the residue. 2 N HCl was then added to the bring material to pH 2. A white precipitate formed filtered and then extracted with EtOAc. The extracts was dried over Na₂SO₄, filtered and evaporated in vacuo. The residue was purified by column chromatography using 30% ethyl acetate/hexane to yield 4-((N-(2-methylcyclohexyl)-6-(trifluoromethyl)pyridine-3-sulfonamido)methyl)benzoic acid, a white solid (605 mg, 97% yield).

Elemental Analysis: (C₂₁H₂₃F₃N₂O₄S):

Calcd: C, 55.25%, H, 5.08%, N, 6.14%. Found C, 55.44%, H, 5.08%, N, 6.14%.

Mp 117-119° C.

MS (m/z): 456.1

¹H NMR (500 MHz, CDCl₃) δ 9.02 (s, 1H), δ 8.09 (d, J=13 Hz, 1H), δ 8.01 (d, J=22 Hz, 2H), δ7.70 (d, J=14 Hz, 1H), δ 7.46 (d, J=14 Hz, 2H), δ 4.49 (d, J=26 Hz, 1H), δ 4.38 (d, J=26 Hz, 1H), δ 1.74 (m, 2H), δ 1.61 (m, 2H), δ 1.27 (m, 3H), δ 1.03 (m, 2H), 0.68 (d, J=11 Hz, 3H)

### Example 33 (AD1196)

### 2-Methyl-2-(4-((N-(2-methylcyclohexyl)-6-(trifluoromethyl)pyridine-3-sulfonamido)methyl)benzamido)propanoicacid

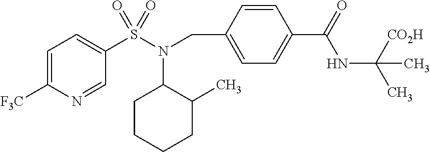 [SMILES: CC1CCCCC1N(Cc1ccc(C(=O)NC(C)(C)C(=O)O)cc1)S(=O)(=O)c1ccc(C(F)(F)F)nc1]

### Step 1

### Methyl 2-methyl-2-(4-((N-(2-methylcyclohexyl)-6-(trifluoromethyl)pyridine-3-sulfonamido)methyl)benzamido)propanoate

4-((N-(2-Methylcyclohexyl)-6-(trifluoromethyl)pyridine-3-sulfonamido)methyl)benzoic acid (200 mg, 0.438 mmol), methyl 2-amino-2-methylpropanoate hydrochloride (135 mg, 0.876 mmol), BOP (484 mg, 1.095 mmol) and HOBt-H₂O (156 mg, 1.095 mmol) were stirred in 2 mL of DMF at room temperature. N-Ethyl-N-isopropylpropan-2-amine (0.382 ml, 2.191 mmol) was added dropwise. The resulting clear solution was stirred at room temperature for 16 h. The solvent was then evaporated in vacuo and 10 mL of water was added to the residue and then extracted with EtOAc. After evaporating the solvent in vacuo, the residue was purified by column chromatography using 20% ethyl acetate/hexane to give methyl 2-methyl-2-(4-((N-(2-methylcyclohexyl)-6-(trifluoromethyl)pyridine-3-sulfonamido)methyl)benzamido)propanoate, a white solid (180 mg, 74% yield).

Mp 108-110° C.

### Step 2

### 2-Methyl-2-(4-((N-(2-methylcyclohexyl)-6-(trifluoromethyl)pyridine-3-sulfonamido)methyl)benzamido)propanoic acid

Methyl 2-methyl-2-(4-((N-(2-methylcyclohexyl)-6-(trifluoromethyl)pyridine-3-sulfonamido)methyl)benzamido)propanoate (240 mg, 0.432 mmol) and lithium hydroxide hydrate (54.4 mg, 1.296 mmol) were stirred in THF (2 mL) and H2O (1 mL) overnight at 50° C. The mixture was then cooled to room temperature and THF was evaporated in vacuo. Water (10 mL) was added to the residue and then 2 N HCl was added until a pH 2 was reached. The mixture was then extracted with EtOAc, dried over Na₂SO₄, filtered, and evaporated in vacuo to give crude product. Purification by column chromatography (30% ethyl acetate in hexane) yielded 2-methyl-2-(4-((N-(2-methylcyclohexyl)-6-(trifluoromethyl)pyridine-3-sulfonamido)methyl)benzamido)propanoic acid as a white solid (170 mg, 70% yield).

Mp 131-132° C.

MS (m/z): 541.2

¹H NMR (500 MHz, CDCl₃) δ 11.80 (s, 1H), δ 8.86 (s, 1H), δ 8.19 (d, J=1.5 Hz, 1H), δ 8.06 (s, 1H), δ 7.73 (d, J=7 Hz, 1H), δ 7.44 (d, J=7 Hz, 2H), δ 7.14 (d, J=7 Hz, 2H), δ 4.14 (d, J=13 Hz, 2H), δ 2.95 (s, 3H), δ 2.14 (s, 3H), δ 1.24 (m, 2H), δ 1.10 (m, 2H), δ 0.94 (m, 4H), δ 0.94 (s, 2H), δ 0.10 (d, J=5 Hz, 3H)

### Example 34 (AD1198)

### 2-(4-((4-Chloro-N-(2-methylcyclohexyl)phenylsulfonamido)methyl)benzamido)-2-methylpropanoic acid

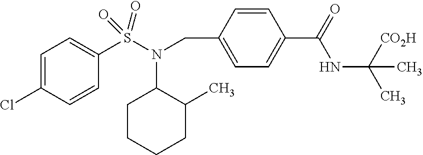 [SMILES: CC1CCCCC1N(Cc1ccc(C(=O)NC(C)(C)C(=O)O)cc1)S(=O)(=O)c1ccc(Cl)cc1]

### Step 1

### Methyl 2-(4-((4-chloro-N-(2-methylcyclohexyl)phenylsulfonamido)methyl)benzamido)-2-methylpropanoate

4-((4-Chloro-N-(2-methylcyclohexyl)phenylsulfonamido)methyl)benzoic acid (560 mg, 1.327 mmol), methyl 2-amino-2-methylpropanoate hydrochloride (408 mg, 2.65 mmol), BOP (1468 mg, 3.32 mmol) and HOBt-H2O (448 mg, 3.32 mmol) were stirred in DMF at room temperature. N-Ethyl-N-isopropylpropan-2-amine (1.156 ml, 6.64 mmol) was added dropwise to this mixture and then stirred for 16 h at room temperature. The solvent was then evaporated in vacuo to which 20 mL of water was added to the residue and then extracted with EtOAc. EtOAc was removed in vacuo and the crude product was purified by column chromatography using 20% ethyl acetate/hexane to give methyl 2-(4-((4-chloro-N-(2-methylcyclohexyl)phenylsulfonamido)methyl)benzamido)-2-methylpropanoate, a white solid (544 mg, 78% yield).

Mp 98-100° C.

### Step 2

### 2-(4-((4-Chloro-N-(2-methylcyclohexyl)phenylsulfonamido)methyl)benzamido)-2-methylpropanoic acid

Methyl 2-(4-((4-chloro-N-(2-methylcyclohexyl)phenylsulfonamido)methyl)benzamido)-2-methylpropanoate (544 mg, 1.044 mmol) and lithium hydroxide hydrate (131 mg, 3.13 mmol) were stirred in a mixture of THF (4 mL) and H₂O (2 mL) at 50° C. for 16 h. After cooling to room temperature, the reaction mixture was concentrated in vacuo to remove the THF and then 10 mL of water was added to the residue. This residue was extracted with 10 mL of diethyl ether to remove impurities, and then the residue was adjusted to pH 2 using 2 N HCl. Extraction then with EtOAc, followed by evaporating in vacuo gave a white solid that was stirred in 10 mL of diethyl ether to give 2-(4-((4-chloro-N-(2-methylcyclohexyl)phenylsulfonamido)methyl)benzamido)-2-methylpropanoic acid (356 mg, 67% yield).

Elemental Analysis: (C₂₅H₃₁ClN₂O₅S):

Calcd: C, 59.22%, H, 6.16%, N, 5.52%. Found C, 59.21%, H, 6.16%, N, 5.48%

Mp 121-122° C.

MS (m/z): 506.1

¹H NMR (500 MHz, CDCl₃) δ 7.69 (m, 4H), δ 7.48 (m, 2H), δ 7.43 (m, 2H), δ 6.59 (s, 1H), δ 4.37 (d, J=8 Hz, 2H), δ 4.20 (d, J=4.5 Hz, 1H), δ 3.34 (s, 1H), δ 1.73 (s, 6H), δ 1.66 (m, 1H), δ 1.55 (m, 1H), δ 1.47 (m, 1H), δ 1.31 (m, 1H), δ 1.18 (m, 1H), δ 0.96 (m, 1H), δ 0.87 (m, 2H), δ 0.59 (d, J=8 Hz, 3H)

### Example 35 (AD1201)

### Methyl 3-(4-((4-chloro-N-(2-methylcyclohexyl)phenylsulfonamido)methyl)benzamido)propanoate

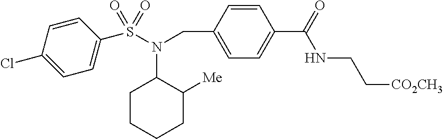 [SMILES: COC(=O)CCNC(=O)c1ccc(CN(C2CCCCC2C)S(=O)(=O)c2ccc(Cl)cc2)cc1]

Methyl 3-(4-((4-chloro-N-(2-methylcyclohexyl)phenylsulfonamido)methyl)benzamido)propanoate was prepared via the General Method described in Scheme 1, using Example 3 and ethyl 3-aminopropanoate hydrochloride. The desired product was isolated in a yield of 77%.

MS (m/z): 520.02

Elemental Analysis: (C₂₅H₃₁ClN₂O₅S):

Calcd: C, 59.22%; H, 6.16%; N, 5.52%. Found: C, 59.0%; H, 6.35%; N, 5.3%

### Example 36 (AD1202)

### 3-(4-((4-Chloro-N-(2-methylcyclohexyl)phenylsulfonamido)methyl)benzamido)propanoic acid

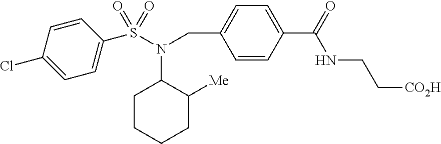 [SMILES: CC1CCCCC1N(Cc1ccc(C(=O)NCCC(=O)O)cc1)S(=O)(=O)c1ccc(Cl)cc1]

3-(4-((4-Chloro-N-(2-methylcyclohexyl)phenylsulfonamido)methyl)benzamido)propanoic acid was hydrolyzed from Example 35 using 2 M NaOH and THF and isolated in a yield of 85%.

MS (m/z): 493

Elemental Analysis: (C₂₄H₂₉ClN₂O₅S):

Calcd: C, 58.47%; H, 5.93%; N, 5.68%. Found: C, 58.37%; H, 5.79%; N, 5.96%

### Example 37 (AD1204)

### 4-((4-Chloro-N-(2-methylcyclohexyl)phenylsulfonamido)methyl)-N-(1-hydroxy-2-methylpropan-2-yl)benzamide

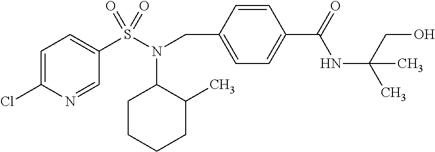 [SMILES: CC1CCCCC1N(Cc1ccc(C(=O)NC(C)(C)CO)cc1)S(=O)(=O)c1ccc(Cl)nc1]

4-((4-Chloro-N-(2-methylcyclohexyl)phenylsulfonamido)methyl)benzoic acid (80 mg, 0.19 mmol), 2-amino-2-methylpropan-1-ol (37.6 mg, 0.38 mmol), BOP (109 mg, 0.25 mmol) and HOBt-H2O (33.3 mg, 0.25 mmol) were stirred in DMF at room temperature and then N-ethyl-N-isopropylpropan-2-amine (0.132 ml, 0.76 mmol) was added dropwise. The mixture was stirred for 16 h at room temperature. After evaporating all solvent and adding 10 mL of water, the mixture was then extracted with EtOAc. The combined organic layers were concentrated in vacuo to give crude product which was purified by column chromatography using 20% ethyl acetate/hexane to give 4-((4-chloro-N-(2-methylcyclohexyl)phenylsulfonamido)methyl)-N-(1-hydroxy-2-methylpropan-2-yl)benzamide, a white solid (20 mg, 21% yield).

Mp 116-117° C.

MS (m/z): 474.1 (M-H₂O)

¹H NMR (500 MHz, CDCl₃) δ 7.70 (m, 2H), δ 7.66 (m, 2H), δ 7.43 (m, 4H), δ 4.37 (d, J=8 Hz, 2H), δ 4.20 (m, 1H), δ 3.71 (d, J=5 Hz, 2H), δ 1.70 (m, 1H), δ 1.55 (m, 1H), δ 1.42 (s, 6H), δ 1.20-1.40 (m, 2H), δ 0.87 (m, 1H), δ 0.75 (m, 1H), δ 0.59 (m, 2H)

### Example 38 (AD1206)

### 4-((4-Chloro-N-(2-methylcyclohexyl)phenylsulfonamido)methyl)-N-(2-(pyrrolidin-1-yl)ethyl)benzamide

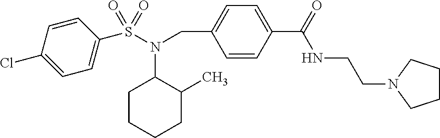 [SMILES: CC1CCCCC1N(Cc1ccc(C(=O)NCCN2CCCC2)cc1)S(=O)(=O)c1ccc(Cl)cc1]

4-((4-Chloro-N-(2-methylcyclohexyl)phenylsulfonamido)methyl)-N-(2-(pyrrolidin-1-yl)ethyl)benzamide was prepared from Example 3 and 2-(pyrrolidin-1-yl)ethanamine in a yield of 70.6%.

MS (m/z): 518

Elemental Analysis: (C₂₇H₃₆ClN₃O₃S):

Calcd: C, 62.59%; H, 7.00%; N, 8.11%. Found: C, 59.94%; H, 7.02%; N, 7.56%

### Example 39 (AD1207)

### 4-((4-Chloro-N-(2-methylcyclohexyl)phenylsulfonamido)methyl)-N-ethyl-N-(2-(pyrrolidin-1-yl)ethyl)benzamide

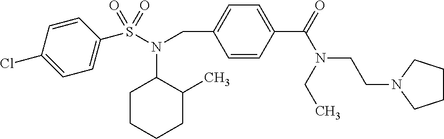 [SMILES: CCN(CCN1CCCC1)C(=O)c1ccc(CN(C2CCCCC2C)S(=O)(=O)c2ccc(Cl)cc2)cc1]

4-((4-Chloro-N-(2-methylcyclohexyl)phenylsulfonamido)methyl)-N-ethyl-N-(2-(pyrrolidin-1-yl)ethyl)benzamide was prepared via the General Method described in Scheme 1, using Example 3 and N-ethyl-2-(pyrrolidin-1-yl)ethanamine. The yield was 49.4%.

MS (m/z): 545.3

Elemental Analysis: (C₂₉H₄₀ClN₃O₃S):

Calcd: C, 63.77%; H, 7.38%; N, 7.69%. Found: C, 62.38%; H, 7.21%; N, 7.96%

### Example 40 (AD1208)

### 4-((4-Chloro-N-(2-methylcyclohexyl)phenylsulfonamido)methyl)-N-(2-(dimethylamino)ethyl)benzamide

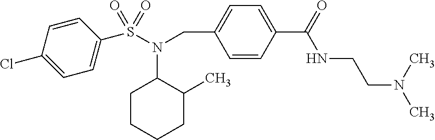 [SMILES: CC1CCCCC1N(Cc1ccc(C(=O)NCCN(C)C)cc1)S(=O)(=O)c1ccc(Cl)cc1]

4-((4-Chloro-N-(2-methylcyclohexyl)phenylsulfonamido)methyl)-N-(2-(dimethylamino)ethyl)benzamide was prepared via the General Method described in Scheme 1, using Example 3 and N₁,N₁-dimethylethane-1,2-diamine (31.3 mg, 0.356 mmol). The yield was 72%.

MS (m/z): 489.1

Elemental Analysis: (C₂H₂ClNO₆S):

Calcd: C, 58.62%; H, 6.96%; N, 5.54%. Found: C, 58.02%; H, 8.25%; N, 6.80%

### Example 41 (AD1209-2)

### 4-((4-Chloro-N-(2-methylcyclohexyl)phenylsulfonamido)methyl)-N-(2-(dimethylamino)ethyl)benzamide hydrochloride

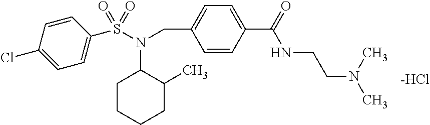 [SMILES: CC1CCCCC1N(Cc1ccc(C(=O)NCCN(C)C)cc1)S(=O)(=O)c1ccc(Cl)cc1 ; "-HCl"]

4-((4-Chloro-N-(2-methylcyclohexyl)phenylsulfonamido)methyl)-N-(2-(dimethylamino)ethyl)benzamide hydrochloride was prepared from Example 40 and hydrochloride in diethyl ether. The solid salt was collected and washed with cold diethyl ether and dried. The yield was 40%.

MS (m/z): 492.5

Elemental Analysis: (C₂₅H₃₅Cl₂N₃O₃S):

Calcd: C, 56.81%; H, 6.67%; N, 7.95%. Found: C, 53.83%; H, 7.71%; N, 8.20%

### Example 42 (AD1211)

### Methyl 4-((4-chloro-N-(3-methylcyclohexyl)phenylsulfonamido)methyl)benzoate

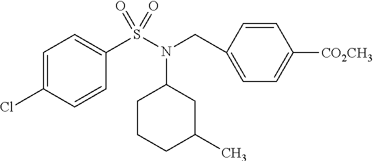 [SMILES: COC(=O)c1ccc(CN(C2CCCC(C)C2)S(=O)(=O)c2ccc(Cl)cc2)cc1]

### Step 1

### 4-Chloro-N-(3-methylcyclohexyl)benzenesulfonamide

To a solution of 4-chlorobenzene-1-sulfonyl chloride (0.600 g, 2.84 mmol) in dichloromethane (10 mL) was added 3-methylcyclohexylamine (0.322 g, 2.84 mmol) followed by triethylamine (0.791 ml, 5.69 mmol). The reaction mixture was stirred at room temperature for 3 h, washed with water, dried with sodium sulfate and purified by column chromatography (silica gel) eluting with ethyl acetate/hexane (0 to 50%) to give 4-chloro-N-(3-methylcyclohexyl)benzenesulfonamide (0.720 g, 88%) as a white solid.

### Step 2

### 4-((4-Chloro-N-(3-methylcyclohexyl)phenylsulfonamido)methyl)benzoate

To a mixture of 4-chloro-N-(3-methylcyclohexyl)benzenesulfonamide (0.700 g, 2.432 mmol) and methyl 4-(bromomethyl)benzoate (0.557 g, 2.432 mmol) in acetonitrile (20 mL) was added cesium carbonate (1.579 g, 4.85 mmol). The reaction mixture was stirred at room temperature for 4 h and then 20 mL of water was added to the mixture. The mixture was then extracted with ethyl acetate and the organic layer was separated, washed with water and brine and then dried using sodium sulfate and filtered. The solvent was evaporated in vacuo and the crude product was purified using ethyl acetate/hexane (0% to 40%, silica gel) to yield methyl 4-((4-chloro-N-(3-methylcyclohexyl)phenylsulfonamido)methyl)benzoate (0.980 g, 92% yield).

MS (m/z): 435.9

### Example 43 (AD1213)

### 4-((4-Chloro-N-(3-methylcyclohexyl)phenylsulfonamido)methyl)benzoic acid

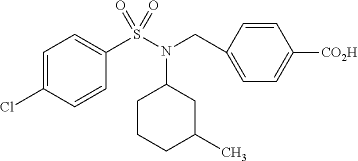 [SMILES: CC1CCCC(N(Cc2ccc(C(=O)O)cc2)S(=O)(=O)c2ccc(Cl)cc2)C1]

To a solution of methyl 4-((4-chloro-N-(3-methylcyclohexyl)phenylsulfonamido)methyl)benzoate (0.800 g, 1.835 mmol, Example 42) in acetonitrile (10 mL) was added 1 mL of lithium hydroxide hydrate (0.308 g, 7.34 mmol) aqueous solution. The mixture was stirred at room temperature for 16 h. 4 N HCl was then added to bring the reaction mixture to pH 2.5. The solvent was then evaporated and the residue was dissolved in ethyl acetate and washed with water, brine and dried with sodium sulfate. Filtration and concentration in vacuo provided 4-((4-chloro-N-(3-methylcyclohexyl)phenyl-sulfonamido)methyl)benzoic acid (0.550 g, 71%) as a white solid that was triturated with a small amount of diethyl ether.

MS (m/z): 422.9

¹H NMR (400 MHz, DMSO-d₆) δ 12.85 (H, s), 7.95 (4H, dd), 7.68 (2H, d), 7.42 (2H, d), 4.52 (2H, s), 3.78 (1H, m), 1.60-0.86 (9H, m), 0.70 (3H, d)

### Example 44 (AD1212)

### 4-((6-Chloro-N-(2-methylcyclohexyl)pyridine-3-sulfonamido)methyl)benzoic acid

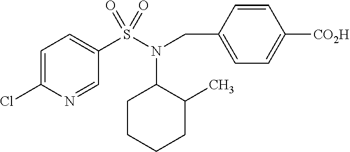 [SMILES: CC1CCCCC1N(Cc1ccc(C(=O)O)cc1)S(=O)(=O)c1ccc(Cl)nc1]

4-((6-Chloro-N-(2-methylcyclohexyl)pyridine-3-sulfonamido)methyl)benzoic acid (0.300 g, 73%) was obtained using the same procedure illustrated for Examples 42 and 43.

MS (m/z): 422.9

¹H NMR (400 MHz, DMSO-d₆) δ (12.85 (H, s), 8.90 (1H, s), 8.39 (1H, d), 7.95 (2H, dd), 7.85 (1H, d), 7.44 (2H, d), 4.45 (2H, s), 3.98 (1H, m), 1.60-0.86 (9H, m), 0.80 (3H, d)

### Example 45 (AD1216)

### Methyl 4-((4-chloro-N-cyclohexylphenylsulfonamido)methyl)benzoate 4-((4-chloro-N-cyclohexylphenylsulfonamido)methyl)benzoic acid

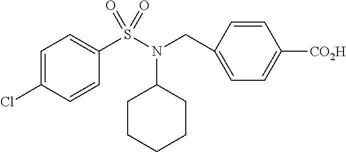 [SMILES: O=C(O)c1ccc(CN(C2CCCCC2)S(=O)(=O)c2ccc(Cl)cc2)cc1]

Methyl 4-((4-chloro-N-cyclohexylphenylsulfonamido)methyl)benzoate (1.2 g, 2.84 mmol) and lithium hydroxide hydrate (0.358 g, 8.53 mmol) were stirred in THF (10 mL) and water (5 mL) at 50° C. for 16 h. THF was evaporated in vacuo and 20 mL of ether was added to the residue to remove impurities. Water (20 mL) was then added and the aqueous phase was adjusted to pH 2 using a 6 N HCl solution. The mixture was then extracted with EtOAc, dried and concentrated in vacuo to give 4-((4-chloro-N-cyclohexylphenylsulfonamido) methyl)benzoic acid, an off-white solid (1.02 g, 88% yield).

Mp 135-137° C.

MS (m/z): 407.1

¹H NMR (500 MHz, CDCl₃) δ 8.07 (d, J=1.5 Hz, 2H), δ 7.74 (d, J=4 Hz, 2H), δ 7.47 (m, 4H), δ 4.47 (s, 2H), δ 3.75 (m, 1H), δ 1.67 (m, 2H), δ 1.52 (m, 3H), δ 1.17 (m, 4H), δ 0.90 (m, 1H)

### Example 46 (AD1221)

### 4-((4-Chloro-N-(2-methylcyclohexyl)phenylsulfonamido)methyl)-N-((R)-1-methoxypropan-2-yl)benzamide

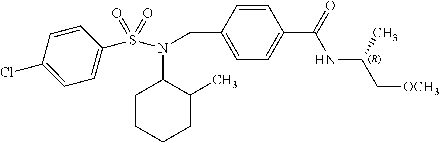

4-((4-Chloro-N-(2-methylcyclohexyl)phenylsulfonamido)methyl)-N—((R)-1-methoxypropan-2-yl)benzamide was prepared from Example 4 and (R)-1-methoxypropan-2-amine as described in the General Methods, Scheme 1. Final product was isolated in 70% yield.

MS (m/z): 493.4

Elemental Analysis: (C₂₅H₃₃ClN₂O₄S):

Calcd: C, 60.99%; H, 6.75%; N, 5.68%. Found: C, 62.05%; H, 6.75%; N, 5.80%

### Example 47 (AD1222)

### 4-((4-Chloro-N-(2-methylcyclohexyl)phenylsulfonamido)methyl)-N—((S)-2-hydroxypropyl)benzamide

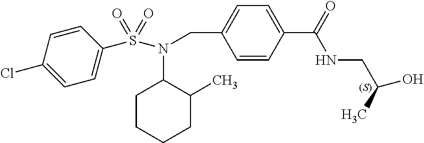

4-((4-Chloro-N-(2-methylcyclohexyl)phenylsulfonamido)methyl)-N—((S)-2-hydroxypropyl)benzamide was prepared from Example 3 and (S)-1-aminopropan-2-ol using the General Method described in Scheme 1. Desired product was obtained in a yield of 59.7%.

MS (m/z): 477.4

Elemental Analysis: (C₂₄H₃₁ClN₂O₄S):

Calcd: C, 60.17%; H, 6.52%; N, 5.85%. Found: C, 61.85%; H, 6.47%; N, 5.73%

### Example 48 (AD1223)

### 4-((4-Chloro-N-(2-methylcyclohexyl)phenylsulfonamido)methyl)-N-(2-oxopropyl)benzamide

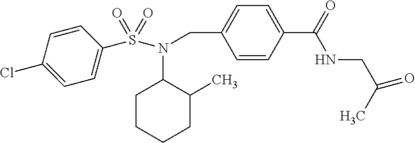 [SMILES: CC(=O)CNC(=O)c1ccc(CN(C2CCCCC2C)S(=O)(=O)c2ccc(Cl)cc2)cc1]

Oxidation of Example 47 with chromium trioxide/pyridine afforded 4-((4-chloro-N-(2-methylcyclohexyl)phenylsulfonamido)methyl)-N-(2-oxopropyl)benzamide, the desired product in a yield of 40%.

MS (m/z): 477.0

Elemental Analysis: (C₂H₂ClNO₆S):

Calcd: C, 60.43%; H, 6.13%; N, 5.87%. Found: C, 60.37%; H, 6.23%; N, 5.73%

### Example 49 (AD1225)

### Methyl 4-((4-chloro-N-(3-(trifluoromethyl)cyclohexyl)phenylsulfonamido)methyl)benzoate

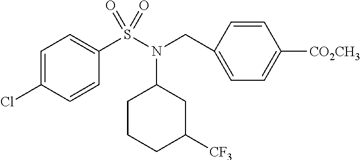 [SMILES: COC(=O)c1ccc(CN(C2CCCC(C(F)(F)F)C2)S(=O)(=O)c2ccc(Cl)cc2)cc1]

Methyl 4-((4-chloro-N-(3-(trifluoromethyl)cyclohexyl)phenylsulfonamido)methyl)benzoate (0.600 g, 69.8%) was obtained using the same procedure as described for Example 42.

MS (m/z): 490.1

### Example 50 (AD1226)

### 4-((4-Chloro-N-(3-methylcyclohexyl)phenylsulfonamido)methyl)-N—((R)-1-hydroxypropan-2-yl)benzamide

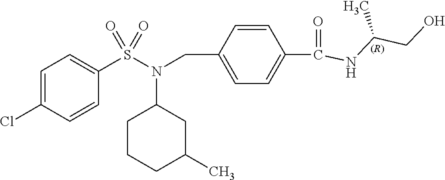

To a solution of 4-((4-chloro-N-(3-methylcyclohexyl)phenylsulfonamido)methyl)benzoic acid (0.15 g, 0.356 mmol) (Example 43) and (R)-2-aminopropan-1-ol (0.027 g, 0.356 mmol) in DMF (2 mL) was added EDCI (0.075 g, 0.391 mmol) and DMAP (4.34 mg, 0.036 mmol). The reaction mixture was stirred at room temperature for 4 h. After the solvent was evaporated in vacuo, the residue was passed through silica gel by eluting with ethyl acetate/hexane (50 to 100%) to obtain 4-((4-chloro-N-(3-methylcyclohexyl)phenylsulfonamido) methyl)-N—((R)-1-hydroxypropan-2-yl)benzamide (0.100 g, 58.7%) as a white solid.

MS (m/z): 479.1

¹NMR (400 MHz, DMSO-d₆) δ 8.05 (H, d), 7.90 (2H, d), 7.80 (2H, d), 7.70 (2H, d), 7.40 (2H, d), 4.7 (1H, m), 4.45 (2H, s), 4.00 (1H, m), 3.50 (1H, m), 3.30 (1H, m), 1.60-0.86 (12H, m), 0.78 (3H, d)

### Example 51 (AD1227)

### 4-((4-Chloro-N-(3-(trifluoromethyl)cyclohexyl)phenylsulfonamido)methyl)benzoic acid

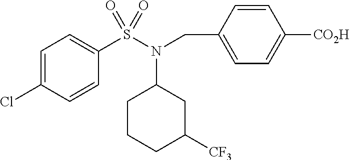 [SMILES: O=C(O)c1ccc(CN(C2CCCC(C(F)(F)F)C2)S(=O)(=O)c2ccc(Cl)cc2)cc1]

4-((4-Chloro-N-(3-(trifluoromethyl)cyclohexyl)phenylsulfonamido)methyl)benzoic acid (0.300 g, 73.5%) was obtained by the same procedure as that described for Example 43.

MS (m/z): 476.1

Elemental Analysis: C₂₁H₂₁ClF₃NO₄S:

Calcd: C, 53.00%; H, 4.45%; N, 2.94%. Found: C, 52.77%; H, 4.19%; N, 2.84%

### Example 52 (AD1227C) and Example 53 (AD1227D)

### 4-((4-Chloro-N-(3-(trifluoromethyl)cyclohexyl)phenylsulfonamido)methyl)benzoic acid

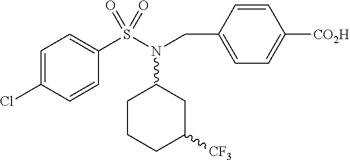 [SMILES: O=C(O)c1ccc(CN(C2CCCC(C(F)(F)F)C2)S(=O)(=O)c2ccc(Cl)cc2)cc1]

The chiral separation of Example 51 gave two major products: Example 52 (42%, Retention time=2.96 min) and Example 53 (48%, Retention time=3.85 min) The separation conditions used were: Preparative-SFC conditions [(Instrument: SFC-80 Thar Technologies, Waters, USA; Column. Chiralpak AD-H 30*250 mm, 5 μm Daicel Industries, Japan; Column temperature: 35° C.; Mobile phase: CO₂/MeOH=70/30; Flow rate: 80 g/min; Back pressure: 100 bar; Detection wavelength: 214 nm; Cycle time: 6.3 min; Feed solution: 140 mg sample dissolved in 20 mL MeOH; Injection volume: 1.5 mL (loading: 10.5 mg/injection)]

### Example 54 (AD1228)

### 4-((4-Chloro-N-(2-methylcyclohexyl)phenylsulfonamido)methyl)-N-(2,2,2-trifluoroethyl)benzamide

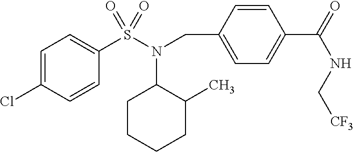 [SMILES: CC1CCCCC1N(Cc1ccc(C(=O)NCC(F)(F)F)cc1)S(=O)(=O)c1ccc(Cl)cc1]

4-((4-Chloro-N-(2-methylcyclohexyl)phenylsulfonamido)methyl)-N-(2,2,2-trifluoroethyl)benzamide (0.150 g, 62.9%) was obtained using the same procedure as that described for the preparation of Example 50.

MS (m/z): 503.3

Elemental Analysis: C₂₃H₂₆ClF₃N₂O₃S:

Calcd: C, 54.92%; H, 5.21%; N, 5.57%. Found: C, 54.82%; H, 5.59%; N, 5.43%

### Example 55 (AD1229)

### 4-((4-Chloro-N-(2-methylcyclohexyl)phenylsulfonamido)methyl)-N-(3,3,3 trifluoropropyl)benzamide

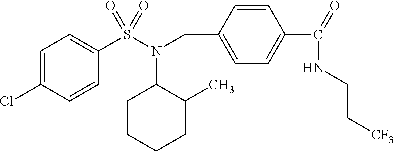 [SMILES: CC1CCCCC1N(Cc1ccc(C(=O)NCCC(F)(F)F)cc1)S(=O)(=O)c1ccc(Cl)cc1]

4-((4-Chloro-N-(2-methylcyclohexyl)phenylsulfonamido)methyl)-N-(3,3,3 trifluoropropyl)benzamide (0.150 g, 61.2%) was obtained using the same procedure as that described for the preparation of Example 50.

MS (m/z): 517.3

Elemental Analysis: C₂₄H₂₈ClF₃N₂O₃S

Calcd: C, 55.76%; H, 5.46%; N, 5.42%. Found: C, 55.67%; H, 5.63%; N, 5.07%

### Example 56 (AD1230)

### Methyl 4-((4-chloro-N-(2-fluorocyclohexyl)phenylsulfonamido)methyl)benzoate

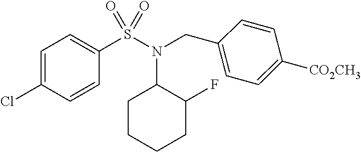 [SMILES: COC(=O)c1ccc(CN(C2CCCCC2F)S(=O)(=O)c2ccc(Cl)cc2)cc1]

### Step 1

### Methyl 4-((2-fluorocyclohexyl)amino)methyl)benzoate

Using the synthetic procedure outlined in General Scheme 2,2-fluoro-cyclohexanone (2.00 g, 17.22 mmol) and methyl 4-(aminomethyl)benzoate (3.47 g, 17.22 mmol) were stirred at room temperature in dichloroethane (50 mL) for 5 minutes. Sodium triacetoxyhydroborate (5.11 g, 24.11 mmol) was then added and the reaction mixture was then stirred at room temperature for 16 h. Water was added to reaction mixture and stirred for 5 min. Layers were separated and the organic layer was washed with NaHCO₃(s), water, dried with sodium sulfate and concentrated in vacuo to give crude product which was purified by column chromatography (silica gel) using ethyl acetate/hexane (0 to 100%). The desired product, methyl 4-(((2-fluorocyclohexyl)amino)methyl)benzoate (3.20 g, 67%), was isolated as a clear oil.

MS (m/z): 275.3

### Step 2

### Methyl 4-((4-chloro-N-(2-fluorocyclohexyl)phenylsulfonamido)methyl)benzoate

To a solution of 4-chlorobenzene-1-sulfonyl chloride (0.550 g, 2.61 mmol) and methyl 4-((2-fluorocyclohexylamino)methyl)benzoate (0.691 g, 2.61 mmol) in dichloromethane (10 mL) was added triethylamine (0.73 ml, 5.21 mmol). The reaction mixture was then stirred at room temperature 16 h. The solvent was then concentrated in vacuo to give a residue that was washed with water and dried with sodium sulfate. The crude material was then purified by column chromatography (silica gel) eluting with EtOAc/hexane (0-50%) to obtain the desired product, methyl 4-((4-chloro-N-(2-fluorocyclohexyl)phenylsulfonamido)methyl)benzoate (0.700 g, 61.1%) as a white solid.

MS (m/z): 439.9

### Example 57 (AD1231)

### 4-((4-Chloro-N-(2-fluorocyclohexyl)phenylsulfonamido)methyl)benzoic acid

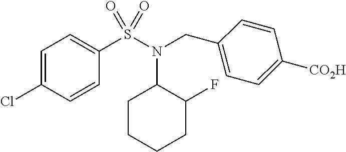 [SMILES: O=C(O)c1ccc(CN(C2CCCCC2F)S(=O)(=O)c2ccc(Cl)cc2)cc1]

To a solution of methyl 4-((4-chloro-N-(2-fluorocyclohexyl)phenylsulfonamido)methyl)benzoate (Example 56, 0.320 g, 0.727 mmol) in acetonitrile (10 mL), lithium hydroxide hydrate (1 mL, 0.122 g, 2.91 mmol) aqueous solution was added. The reaction mixture was stirred at room temperature for 16 h, acidified with 4 N HCl and then extracted with ethyl acetate. The organic layer was then washed with water and brine, dried with sodium sulfate, filtered, and concentrated in vacuo to yield crude product. Triteration with diethyl ether gave the desired product, 4-((4-chloro-N-(2-fluorocyclohexyl)phenylsulfonamido)methyl)benzoic acid (0.310, 87%), as a white solid.

MS (m/z): 425.9

Elemental Analysis: (C₂₀H₂₁ClFNO₄S):

Calcd: C, 56.40%; H, 4.97%; N, 3.29%. Found: C, 58.96%; H, 5.60%; N, 5.57%

### Example 58 (AD1231A) and Example 59 (AD1231B)

### 4-((4-Chloro-N-(2-fluorocyclohexyl)phenylsulfonamido)methyl)benzoic acid

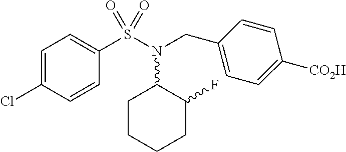 [SMILES: O=C(O)c1ccc(CN(C2CCCCC2F)S(=O)(=O)c2ccc(Cl)cc2)cc1]

The chiral separation of Example 57 gave two major products: Example 58 (42.3%, Retention time=2.3 min) and Example 59 (43.5%, Retention time=2.89 min) The separation conditions used were: Preparative-SFC conditions [(Instrument: SFC-80 Thar Technologies, Waters, USA; Column. Chiralpak AD-H 30*250 mm, 5 μm Daicel Industries, Japan; Column temperature: 35° C.; Mobile phase: CO₂/MeOH=60/40; Flow rate: 70 g/min; Back pressure: 100 bar; Detection wavelength: 214 nm; Cycle time: 5.7 min; Feed solution: 450 mg sample dissolved in 56 mL MeOH; Injection volume: 2.0 mL (loading: 16.1 mg/injection)].

### Example 60 (AD 1235)

### 4-((4-Chloro-N-(2-fluorocyclohexyl)phenylsulfonamido)methyl)-N—((R)-1-hydroxypropan-2-yl)benzamide

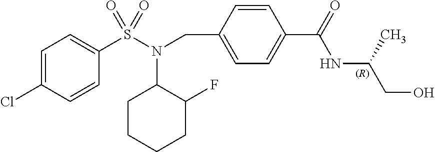

Following the same procedure as that described for Example 50, 4-((4-chloro-N-(2-fluorocyclohexyl)phenylsulfonamido)methyl)benzoic acid (Example 57) was converted to 4-((4-Chloro-N-(2-fluorocyclohexyl)phenylsulfonamido)methyl)-N—((R)-1-hydroxypropan-2-yl)benzamide in 58.8% (0.100 g, 0.207 mmol) yield.

MS (m/z): 483.0

Elemental Analysis: (C₂₃H₂₈ClFN₂O₄S):

Calcd: C, 57.19%; H, 5.84%; N, 5.80%. Found: C, 56.96%; H, 5.60%; N, 5.57%

### Example 61 (AD1240)

### Methyl 4-((6-chloro-N-(2-fluorocyclohexyl)pyridine-3-sulfonamido)methyl)benzoate

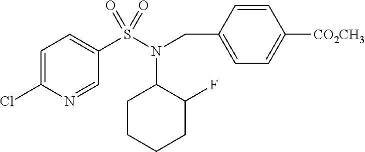 [SMILES: COC(=O)c1ccc(CN(C2CCCCC2F)S(=O)(=O)c2ccc(Cl)nc2)cc1]

Methyl 4-((6-chloro-N-(2-fluorocyclohexyl)pyridine-3-sulfonamido)methyl)benzoate (0.420 g, 67.3%) was prepared by the same method as described for Example 56.

MS (m/z): 440.9

Elemental Analysis: C₂₀H₂₂ClFN₂O₄S

Calcd: C, 54.48%; H, 5.03%; N, 6.35%. Found: C, 54.50%; H, 4.76%; N, 6.14%

### Example 62 (AD1241)

### 4-((6-Chloro-N-(2-fluorocyclohexyl)pyridine-3-sulfonamido)methyl)benzoic acid

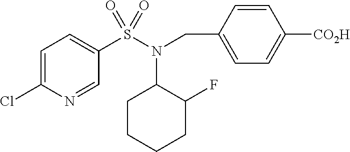 [SMILES: O=C(O)c1ccc(CN(C2CCCCC2F)S(=O)(=O)c2ccc(Cl)nc2)cc1]

4-((6-Chloro-N-(2-fluorocyclohexyl)pyridine-3-sulfonamido)methyl)benzoic acid (0.200 g, 59.0%) was prepared by the same method as described for Example 57.

MS (m/z): 426.9

Elemental Analysis: C₁₉H₂₀ClFN₂O₄S:

Calcd: C, 53.46%; H, 4.72%; N, 6.56%. Found: C, 53.22%; H, 4.60%; N, 6.67%

### Example 63 (AD1242) 4-((6-Chloro-N-(3-(trifluoromethyl)cyclohexyl)pyridine-3-sulfonamido)methyl)benzoic acid

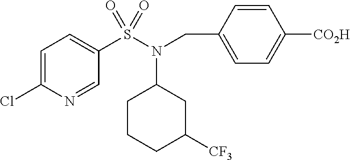 [SMILES: O=C(O)c1ccc(CN(C2CCCC(C(F)(F)F)C2)S(=O)(=O)c2ccc(Cl)nc2)cc1]

4-((6-Chloro-N-(3-(trifluoromethyl)cyclohexyl)pyridine-3-sulfonamido)methyl)benzoic acid (0.250 g, 73.5%) was prepared by the same method as described for Example 43.

MS (m/z): 477.2

Elemental Analysis: C₂₀H₂₀ClF₃N₂O₄S:

Calcd: C, 50.37%; H, 4.23%; N, 5.87%. Found: C, 50.09%; H, 4.01%; N, 5.93%

### Example 64 (AD1243)

### 4-((6-Chloro-N-(3-(trifluoromethyl)cyclohexyl)pyridine-3-sulfonamido)methyl)-N—((R)-1-hydroxypropan-2-yl)benzamide

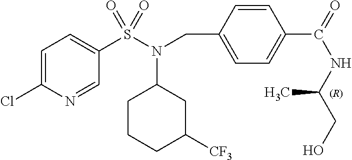

4-((6-Chloro-N-(3-(trifluoromethyl)cyclohexyl)pyridine-3-sulfonamido)methyl)-N—((R)-1-hydroxypropan-2-yl)benzamide (0.160 g, 75%) was prepared by the same method as described for Example 50.

MS (m/z): 534.3

Elemental Analysis: C₂₃H₂₇ClF₃N₃O₄S:

Calcd: C, 51.73%; H, 5.10%; N, 7.87%. Found: C, 51.92%; H, 5.50%; N, 7.64%

### Example 65 (AD1244)

### 4-((6-Chloro-N-(2-fluorocyclohexyl)pyridine-3-sulfonamido)methyl)-N—((R)-1-hydroxypropan-2-yl)benzamide

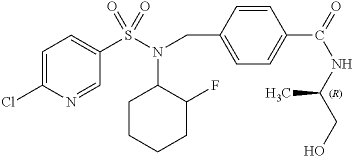

Following the same procedure as that described for Example 60, 4-((6-chloro-N-(2-fluorocyclohexyl)pyridine-3-sulfonamido)methyl)benzoic acid (Example 62) was converted to 4-((6-chloro-N-(2-fluorocyclohexyl)pyridine-3-sulfonamido)methyl)-N—((R)-1-hydroxypropan-2-yl)benzamide in 57.3% (0.130 g) yield.

MS (m/z): 484.3

¹H NMR (400 MHz, DMSO-d₆) δ 9.00 (H, s), 8.40 (H, d), 8.20 (H, d), 7.80 (2H, dd), 7.40 (2H, d), 4.80 (2H, m), 4.60 (2H, s), 4.10 (3H, m), 3.50 (2H, m), 3.40 (H, m), 1.90 (7H, m) 1.10 (3H, d)

### Example 66 (AD1245)

### 4-((4-Chloro-N-(2-fluorocyclohexyl)phenylsulfonamido)methyl)-N-(2-hydroxyethyl)benzamide

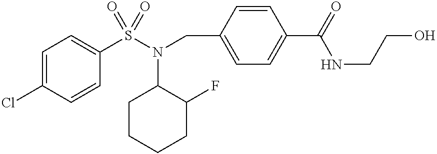 [SMILES: O=C(NCCO)c1ccc(CN(C2CCCCC2F)S(=O)(=O)c2ccc(Cl)cc2)cc1]

Following the same procedure as that described for Example 60, 4-((4-Chloro-N-(2-fluorocyclohexyl)phenylsulfonamido)methyl)benzoic acid (Example 57) was converted to 4-((4-chloro-N-(2-fluorocyclohexyl)phenylsulfonamido)methyl)-N-(2-hydroxyethyl)benzamide in 57.3% (0.130 g) yield.

MS (m/z): 468.3

¹H NMR (400 MHz, DMSO-d₆) δ8.80 (H, s), 8.40 (2H, d), 8.00 (H, d), 7.90 (2H, dd), 7.40 (2H, d), 4.70 (2H, m), 4.60 (2H, s), 4.10 (3H, m), 3.50 (2H, m), 3.40 (H, m), 1.90 (8H, m),

### Example 67 (AD1249)

### 4-((4-Chloro-N-(2-hydroxycyclohexyl)phenylsulfonamido)methyl)-2,3-difluorobenzoic acid

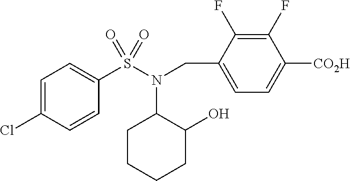 [SMILES: O=C(O)c1ccc(CN(C2CCCCC2O)S(=O)(=O)c2ccc(Cl)cc2)c(F)c1F]

### Step 1

### Methyl 4-((4-chloro-N-(2-hydroxycyclohexyl)phenylsulfonamido)methyl)-2,3-difluorobenzoate

4-Chloro-N-(2-hydroxycyclohexyl)benzenesulfonamide (1.449 g, 5 mmol), methyl 4-(bromomethyl)-2,3-difluorobenzoate (1.104 g, 4.17 mmol) and K₂CO₃ (1.728 g, 12.50 mmol) were stirred in DMF at room temperature for 2 h. TLC showed reaction was complete. Solvent was concentrated in vacuo, 10 mL of water was added to the residue which was then extracted with EtOAc. After evaporating all solvent in vacuo, a white solid resulted which was recrystallized from dichlormethane and methanol to give methyl 4-((4-chloro-N-(2-hydroxycyclohexyl)phenylsulfonamido)methyl)-2,3-difluorobenzoate, the desired product (1.82 g, 76.8% yield).

Mp 123-125° C.

### Step 2

### 4-((4-Chloro-N-(2-hydroxycyclohexyl)phenylsulfonamido)methyl)-2,3-difluorobenzoic acid

Methyl 4-((4-chloro-N-(2-hydroxycyclohexyl)phenylsulfonamido)methyl)-2,3-difluorobenzoate (800 mg, 1.69 mmol) and lithium hydroxide hydrate (142 mg, 3.38 mmol) were stirred at 40° C. for 16 h. The reaction mixture was cooled to room temperature, concentrated in vacuo, and then 8 mL of water was added to the residue and extracted with diethyl ether (3×10 mL) The aqueous phase was adjusted to pH 2 using concentrated HCl solution and then was extracted with EtOAc (3×20 mL) The combined extracts were washed with brine, dried with Na₂SO₄ and evaporated n vacuo to give the desired product, 4-((4-chloro-N-(2-hydroxycyclohexyl)phenylsulfonamido)methyl)-2,3-difluorobenzoic acid as a white solid (720 mg, 92.6% yield).

Mp 134-136° C.

MS (m/z): 459.1

¹H NMR (500 MHz, CDCl₃) δ 7.78 (m, 3H), δ 7.61 (t, J=6 Hz, 1H), δ 7.51 (m, 2H), δ 4.63 (d, J=13 Hz, 1H), δ 4.51 (d, J=13 Hz, 1H), δ 4.20 (d, J=4.5 Hz, 1H), δ 3.52 (m, 1H), δ 3.28 (m, 1H), δ 1.66 (m, 1H), δ 1.27 (m, 3H), δ 1.18 (m, 2H), δ 0.88 (m, 2H)

### Example 68 (AD1250)

### 4-((4-Chloro-N-(2-(trifluoromethyl)cyclohexyl)phenylsulfonamido)methyl)benzoic acid

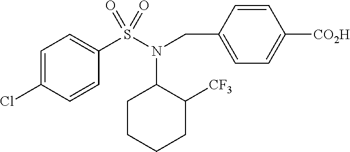 [SMILES: O=C(O)c1ccc(CN(C2CCCCC2C(F)(F)F)S(=O)(=O)c2ccc(Cl)cc2)cc1]

4-((4-Chloro-N-(2-(trifluoromethyl)cyclohexyl)phenylsulfonamido)methyl)benzoic acid (0.145 g, 49.8%) was prepared by the same method as described for Example 43.

MS (m/z): 475.9

Elemental Analysis: C₂₁H₂₁ClF₃NO₄S:

Calcd: C, 53.0%; H, 4.45%; N, 2.94%. Found: C, 51.92%; H, 5.50%; N, 3.11%

### Example 69 (AD1251)

### 4-((4-Chloro-N-(2-(trifluoromethyl)cyclohexyl)phenylsulfonamido)methyl)-N—((S)-1-hydroxypropan-2-yl)benzamide

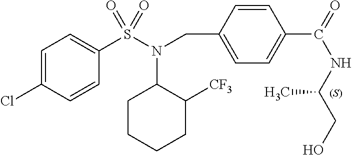

4-((4-Chloro-N-(2-(trifluoromethyl)cyclohexyl)phenylsulfonamido)methyl)-N-((S)-1-hydroxypropan-2-yl)benzamide (0.086 g, 64.2%) was prepared by the same method as described for Example 50.

MS (M/Z): 531

Elemental Analysis: C₂₁H₂₁ClF₃NO₄S:

Calcd: C, 53.0%; H, 4.45%; N, 2.94%. Found: C, 51.92%; H, 5.50%; N, 3.11%

### Example 70 (AD1252)

### Methyl 4-((6-chloro-N-(3-(trifluoromethyl)cyclohexyl)pyridine-3-sulfonamido) methyl)benzoate

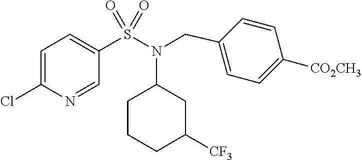 [SMILES: COC(=O)c1ccc(CN(C2CCCC(C(F)(F)F)C2)S(=O)(=O)c2ccc(Cl)nc2)cc1]

Methyl 4-((6-chloro-N-(3-(trifluoromethyl)cyclohexyl) pyridine-3-sulfonamido)methyl)benzoate (0.380 g, 88%) was prepared by the same method as described for Example 42.

MS (m/z): 534.3

### Example 71 (AD1253)

### 4-((4-Chloro-N-cyclohexylphenylsulfonamido)methyl)-2,3-difluorobenzoic acid

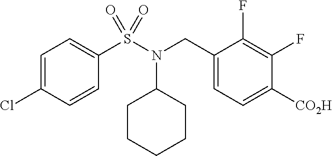 [SMILES: O=C(O)c1ccc(CN(C2CCCCC2)S(=O)(=O)c2ccc(Cl)cc2)c(F)c1F]

### Step 1

### Methyl 4-((4-chloro-N-cyclohexylphenylsulfonamido)methyl)-2,3-difluorobenzoate

To a stirred solution of 4-chloro-N-cyclohexylbenzenesulfonamide (375 mg, 1.370 mmol) and methyl 4-(bromomethyl)-2,3-difluorobenzoate (330 mg, 1.245 mmol) in 6 mL of DMF was added K₂CO₃ (516 mg, 3.74 mmol, 3.0 equiv) at room temperature. After stirring for 16 h, the reaction mixture was quenched with 20 mL of water and extracted with ethyl acetate (2×20 mL) The combined organic extracts were washed with saturated aqueous Na₂CO₃ solution and brine, dried with Na₂SO₄, filtered and concentrated in vacuo to give crude product. Purification by column chromatography using 20% ethyl acetate/hexane yielded 4-chloro-N-(4-cyanobenzyl)-N-(pentan-3-yl)benzenesulfonamide, a white solid (200 mg, 34%).

Mp 131-133° C.

### Step 2

### Methyl 4-((4-chloro-N-cyclohexylphenylsulfonamido)methyl)-2,3-difluorobenzoate

Methyl 4-((4-chloro-N-cyclohexylphenylsulfonamido)methyl)-2,3-difluorobenzoate (200 mg, 0.437 mmol) and lithium hydroxide hydrate (36.7 mg, 0.874 mmol) were stirred in THF (4 mL) and water (2 mL) at room temperature for 16 h. After concentrating the reaction mixture in vacuo, 10 mL of water was added to the residue. This aqueous phase was adjusted to pH 2 using 6 N HCl solution. This acidic solution was then extracted with ethyl acetate and concentrated in vacuo. The residue was extracted with EtOAc, concentrated in vacuo and the resulting crude product was then purified to give
4-((4-chloro-N-cyclohexylphenylsulfonamido)methyl)-2,3-difluorobenzoic acid, a pale white solid (35 mg, 18% yield).

Mp 111-112° C.

MS (m/z): 443.1

1H NMR (500 MHz, CDCl3) δ 7.77 (m, 2H), δ 7.71 (m, 1H), δ 7.51 (m, 3H), δ 4.50 (s, 2H), δ 4.20 (m, 1H), δ 3.79 (m, 1H), δ 1.70 (m, 2H), δ 1.4-1.6 (m, 2H), δ 1.15-1.38 (m, 4H), δ 0.87 (m, 2H)

### Example 72 (AD1254)

### 4-((4-Chloro-N-(2-fluorocyclohexyl)phenylsulfonamido)methyl)-2,3-difluorobenzoic acid

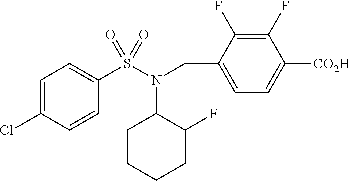 [SMILES: O=C(O)c1ccc(CN(C2CCCCC2F)S(=O)(=O)c2ccc(Cl)cc2)c(F)c1F]

Methyl 4-((4-chloro-N-cyclohexylphenylsulfonamido)methyl)-2,3-difluorobenzoate (200 mg, 0.437 mmol) and lithium hydroxide hydrate (36.7 mg, 0.874 mmol) were stirred in THF (2 mL) and water (1 mL) at room temperature for 16 h. The solvent was then evaporated in vacuo and 10 mL of water was added to the residue. This mixture was adjusted to pH 2 using a 6 N HCl solution. This aqueous phase was then extracted with EtOAc and the resulting crude product was purified to give 4-((4-chloro-N-(2-fluorocyclohexyl)phenylsulfonamido)methyl)-2,3-difluorobenzoic acid, an off-white solid (35 mg, 18% yield).

Mp 108-109° C.

MS (m/z): 461.1

¹H NMR (500 MHz, CDCl₃) δ 7.76 (m, 2H), δ 7.69 (m, 1H), δ 7.51 (m, 3H), δ 4.78 (d, J=14 Hz, 1H), δ 4.62 (d, J=14 Hz, 1H), δ 4.21 (d, J=4.5 Hz, 2H), δ 4.18 (m, 1H), δ 1.70 (m, 1H), δ 1.50 (m, 1H), δ 1.2-1.4 (m, 5H), δ 0.87 (m, 2H)

### Example 73 (AD1255)

### 4-((4-Chloro-N-(2-fluorocyclohexyl)phenylsulfonamido)methyl)-N—((S)-2-hydroxypropyl)benzamide

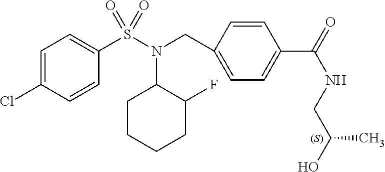

4-((4-Chloro-N-(2-fluorocyclohexyl)phenylsulfonamido)methyl)-N—((S)-2-hydroxypropyl)benzamide (3.48 g, 87.7%) was prepared by the same method as described for Example 50.

MS (m/z): 483.1

Elemental Analysis: C₂₃H₂₈ClFN₂O₄S:

Calcd: C, 57.19%; H, 5.84%; N, 5.80%. Found: C, 57.69%; H, 5.73%; N, 6.23%

### Example 74 (AD 1257)

### 4-((4-Chloro-N-(2-fluorocyclohexyl)phenylsulfonamido)methyl)-N-(2-(dimethylamino)ethyl)benzamide

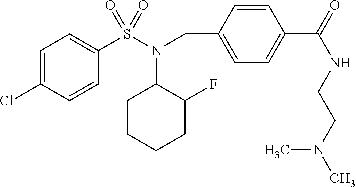 [SMILES: CN(C)CCNC(=O)c1ccc(CN(C2CCCCC2F)S(=O)(=O)c2ccc(Cl)cc2)cc1]

4-((4-Chloro-N-(2-fluorocyclohexyl)phenylsulfonamido)methyl)-N-(2-(dimethylamino)ethyl)benzamide (0.08 g, 68.8%) was prepared by the same method as described for Example 60.

MS (m/z): 496.1

### Example 75 (AD 1257S)

### 4-((4-Chloro-N-(2-fluorocyclohexyl)phenylsulfonamido)methyl)-N-(2-(dimethylamino)ethyl)benzamide hydrochloride

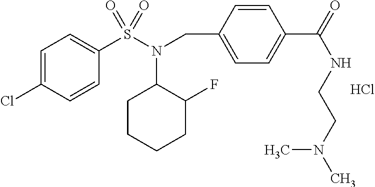 [SMILES: CN(C)CCNC(=O)c1ccc(CN(C2CCCCC2F)S(=O)(=O)c2ccc(Cl)cc2)cc1 ; Cl]

To a solution of 4-((4-chloro-N-(2-fluorocyclohexyl)phenylsulfonamido)methyl)-N-(2-(dimethylamino) ethyl)benzamide (Example 74, 0.120 g, 0.248 mmol) in dioxane (2 mL) was added 1 N HCl in dioxane (4 mL) The reaction mixture was stirred at room temperature for 16 h. The solvent was then evaporated and the resulting residue was washed with ether to obtain the 4-((4-chloro-N-(2-fluorocyclohexyl)phenylsulfonamido)methyl)-N-(2-(dimethylamino)ethyl)benzamide hydrochloride.

MS (m/z): 496.1

### Example 76 (AD1258)

### 4-((4-Chloro-N-(2,2-difluorocyclohexyl)phenylsulfonamido)methyl)benzoic acid

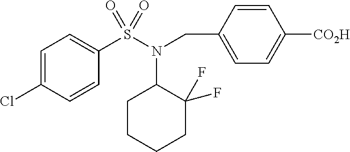 [SMILES: O=C(O)c1ccc(CN(C2CCCCC2(F)F)S(=O)(=O)c2ccc(Cl)cc2)cc1]

Methyl 4-((4-chloro-N-(2-oxocyclohexyl)phenylsulfonamido)methyl)benzoate was prepared via the General Method described in Scheme 1. Further fluorination and hydrolysis of the benzoate afforded 4-((4-chloro-N-(2,2-difluorocyclohexyl)phenylsulfonamido)methyl)benzoic acid in a yield of 40%.

MS (m/z): 444.1 (M⁺+1)

Elemental Analysis: (C₂₀H₂₀ClF₂NO₄S):

Calcd: C, 54.12%; H, 4.54%; N, 3.16%. Found: C, 55.58%; H, 4.61%; N, 3.31%

### Example 77 (AD1259)

### 4-((4-Chloro-N-(2-fluorocyclohexyl)phenylsulfonamido)methyl)-2,3-difluoro-N—((R)-1-hydroxypropan-2-yl)benzamide

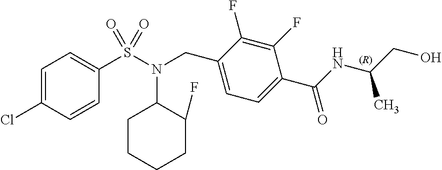

4-((4-Chloro-N-(2-fluorocyclohexyl)phenylsulfonamido)methyl)-2,3-difluorobenzoic acid (140 mg, 0.303 mmol), (R)-2-aminopropan-1-ol (45.5 mg, 0.606 mmol), BOP (174 mg, 0.394 mmol) and HOBt-H2O (56.0 mg, 0.394 mmol) were stirred in DMF at room temperature. N-Ethyl-N-isopropylpropan-2-amine (0.211 ml, 1.212 mmol) was then added and the reaction mixture was stirred at room temperature for 16 h. The mixture was concentrated in vacuo and 10 mL of water was added to the resulting residue. This residue was extracted with EtOAc and the organic layer was then concentrated in vacuo to give crude product. Purification of this crude material by column chromatography (20% ethyl acetate/hexane) gave 4-((4-chloro-N-(2-fluorocyclohexyl)phenylsulfonamido)methyl)-2,3-difluoro-N—((R)-1-hydroxypropan-2-yl)benzamide as a white solid (36 mg, 22% yield).

Mp 126-128° C.

MS (m/z): 518.1

¹H NMR (500 MHz, CDCl₃) δ 7.72 (m, 3H), δ 7.49 (m, 3H), δ 6.64 (m, 1H), δ 4.78 (dd, J1=18 Hz, J2=6 Hz, 1H), δ 4.63 (d, J=18 Hz, 1H), δ 4.73 (s, 1H), δ 4.36 (m, 1H), δ 4.20 (m, 1H), δ3.66 (m, 1H), δ 2.0 (m, 1H), δ 1.80 (m, 2H), δ 1.56 (m, 3H), δ 1.33 (m, 2H), 0.99 (d, J=8 Hz, 3H)

### Example 78 (AD1264)

### 4-Chloro-N-(4-cyanobenzyl)-N-(2-methylcyclohexyl)benzenesulfonamide

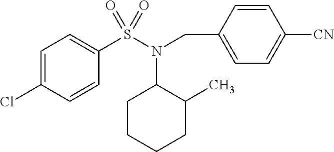 [SMILES: CC1CCCCC1N(Cc1ccc(C#N)cc1)S(=O)(=O)c1ccc(Cl)cc1]

4-Chloro-N-(4-cyanobenzyl)-N-(2-methylcyclohexyl)benzenesulfonamide (0.480 g, 68.6%) was prepared by the same method as described for Example 50.

¹H NMR (400 MHz, DMSO-d₆) δ 7.85 (4H, dd), 7.60 (4H, dd), 4.52 (2H, s), 3.90 (1H, m), 1.60-0.86 (9H, m), 0.50 (3H, d)

### Example 79 (AD 1265)

### 2-(4-((4-Chloro-N-(2-fluorocyclohexyl)phenylsulfonamido)methyl)benzamido)acetic acid

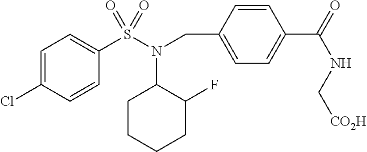 [SMILES: O=C(O)CNC(=O)c1ccc(CN(C2CCCCC2F)S(=O)(=O)c2ccc(Cl)cc2)cc1]

A solution of methyl 2-(4-((4-chloro-N-(2 fluorocyclohexyl)phenylsulfonamido)methyl)benzamido)acetate (0.0800 g, 0.161 mmol; prepared by the General Method shown in Scheme 2) and lithium hydroxide (0.027 g, 0.644 mmol) in acetonitrile (10 mL) and water (1 mL) were stirred at room temperature for 16 h. Water was added and the mixture was extracted with ethyl acetate. The organic layers were separated and washed with water, brine, dried and filtered. The solvent was evaporated in vacuo to give 2-(4-((4-chloro-N-(2-fluorocyclohexyl)phenylsulfonamido)methyl)benzamido)acetic acid (0.0800 g, 64.3%) as the desired product.

MS (m/z): 483.1

Elemental Analysis: C₂₂H₂₄ClFN₂O₅S:

Calcd: C, 54.71%; H, 5.01%; N, 5.80%. Found: C, 54.59%; H, 5.03%; N, 5.53%

### Example 80 (AD 1275S)

### 4-((6-Chloro-N-(2-fluorocyclohexyl)pyridine-3-sulfonamido)methyl)-N-(2-(dimethylamino)ethyl)benzamide hydrochloride

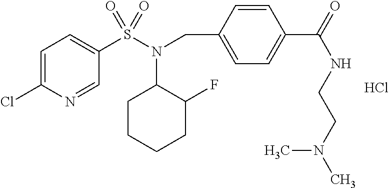 [SMILES: CN(C)CCNC(=O)c1ccc(CN(C2CCCCC2F)S(=O)(=O)c2ccc(Cl)nc2)cc1 ; Cl]

Preparation of 4-((6-chloro-N-(2-fluorocyclohexyl)pyridine-3-sulfonamido)methyl)-N-(2-(dimethylamino)ethyl)benzamide via the General Method described in Scheme 2 followed by formation of its corresponding hydrochloride salt following the procedure described for Example 75 gave 4-((6-chloro-N-(2-fluorocyclohexyl)pyridine-3-sulfonamido)methyl)-N-(2-(dimethylamino)ethyl)benzamide hydrochloride (0.080 g) in 62.3% yield.

MS (m/z): 531.5

### Example 81 (AD1276)

### 4-((4-Chloro-N-(2-chlorocyclohexyl)phenylsulfonamido)methyl)benzoic acid

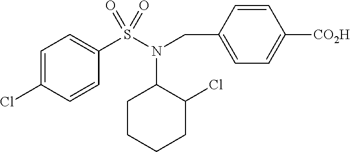 [SMILES: O=C(O)c1ccc(CN(C2CCCCC2Cl)S(=O)(=O)c2ccc(Cl)cc2)cc1]

Methyl 4-((4-chloro-N-(2-chlorocyclohexyl)phenylsulfonamido) methyl)benzoate was prepared as shown in General Methods in Scheme 2. Methyl 4-((4-chloro-N-(2-chlorocyclohexyl)phenylsulfonamido) methyl)benzoate (210 mg, 0.460 mmol) and lithium hydroxide (38.6 mg, 0.920 mmol) were then stirred (THF, 2 mL; H₂O, 1 mL) at 50° C. for 16 h. The reaction mixture was cooled to room temperature and the THF was evaporated in vacuo. Water (10 mL) was added to the residue and the solution was adjusted to pH 2 using 6 N HCl solution. Extraction with EtOAc (3×10 mL), followed by concentration in vacuo gave the crude product that was purified using 40% ethyl acetate/hexane. Purified 4-((4-chloro-N-(2-chlorocyclohexyl)phenylsulfonamido) methyl)benzoic acid was isolated as an off-white solid (100 mg, 49% yield).

Mp 158-160° C.

MS (m/z): 441.1

¹H NMR (600 MHz, CDCl₃) δ 8.06 (d, J=8.4 Hz, 2H), δ 7.77 (m, 2H), δ 7.53 (d, J=6.6 Hz, 2H), δ 7.47 (m, 2H), δ 4.62 (d, J=15.6 Hz, 1H), δ 4.40 (d, J=15.6 Hz, 1H), δ 3.52 (m, 1H), δ 3.21 (m, 1H), δ 2.0 (m, 1H), δ 1.65 (m, 2H), δ 1.50 (m, 1H), δ 1.33 (m, 2H), δ 0.88 (m, 2H)

### Example 82 (AD 1277)

### 4-((4-Chloro-N-(4-fluorocyclohexyl)phenylsulfonamido)methyl)benzoic acid

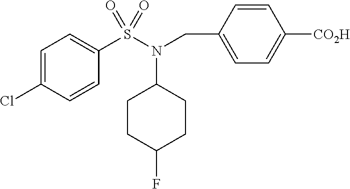 [SMILES: O=C(O)c1ccc(CN(C2CCC(F)CC2)S(=O)(=O)c2ccc(Cl)cc2)cc1]

4-((4-Chloro-N-(4-fluorocyclohexyl)phenylsulfonamido)methyl)benzoic acid (0.098 g, 84%) was prepared by the same method as described for Example 57.

MS (m/z): 426.0

Elemental Analysis: C₂₀H₂₁Cl_{F}NO₄S:

Calcd C, 56.40%; H, 4.97%; N, 3.29%. Found: C, 56.20%; H, 5.15%; N, 3.19%

### Example 83 (AD1278)

### Methyl 4-((4-chloro-N-((1R,2R)-2-fluorocyclohexyl)phenylsulfonamido) methyl)benzoate

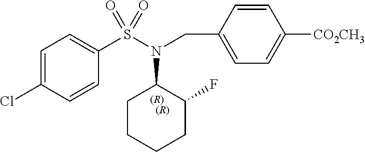

Methyl 4-((4-chloro-N-((1R,2R)-2-fluorocyclohexyl)phenylsulfonamido) methyl)benzoate was prepared via the General Method described in Scheme 1, using the (1R,2R)-2-fluorocyclohexanamine.

MS (m/z): 440.0

Elemental Analysis: (C₂₁H₂₃ClFNO₄S):

Calcd: C, 57.33%; H, 5.27%; N, 3.18 Found: C, 56.89%, H, 5.37%; N, 3.03%

### Example 84 (AD1279)

### 4-((4-Chloro-N-((1R,2R)-2-fluorocyclohexyl)phenylsulfonamido)methyl)benzoic acid

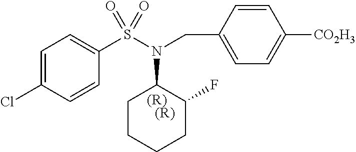

4-((4-Chloro-N-((1R,2R)-2-fluorocyclohexyl)phenylsulfonamido)methyl)benzoic acid was prepared via hydrolysis of Example 83 in a yield of 71%, following the General Method described in Scheme 1.

MS (m/z): 425.5

Elemental Analysis: (C₂₀H₂₁ClFNO₄S):

Calcd: C, 56.40%; H, 4.97%; N, 4.46% Found: C, 55.75%, H, 4.67%, N, 3.14%

### Example 85 (AD 1280)

### 4-((4-Chloro-N-(2-fluorocyclohexyl)phenylsulfonamido)methyl)-N-(1-methoxypropan-2-yl)benzamide

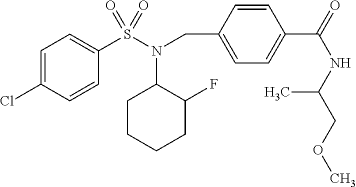 [SMILES: COCC(C)NC(=O)c1ccc(CN(C2CCCCC2F)S(=O)(=O)c2ccc(Cl)cc2)cc1]

4-((4-Chloro-N-(2-fluorocyclohexyl)phenylsulfonamido)methyl)-N-(1-methoxypropan-2-yl)benzamide (0.092 g, 64.3%) was prepared by the same method as described for Example 60.

MS (m/z): 496.9

Elemental Analysis: C₂₄H₃₀ClFN₂O₄S:

Calcd: C, 58.0%; H, 6.08%; N, 5.64%. Found: C, 58.3%; H, 5.93%; N, 5.42%

### Example 86 (AD 1281)

### 4-((4-Chloro-N-(2-fluorocyclohexyl)phenylsulfonamido)methyl)-N—((S)-1-methoxypropan-2-yl)benzamide

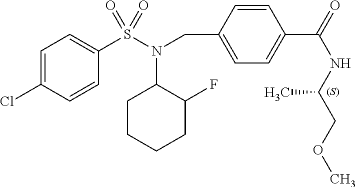

4-((4-Chloro-N-(2-fluorocyclohexyl)phenylsulfonamido)methyl)-N—((S)-1-methoxypropan-2-yl)benzamide (0.092 g, 64.3%) was prepared by the same method as described for Example 60.

MS (m/z): 496.9

Elemental Analysis: C₂₄H₃₀ClFN₂O₄S:

Calcd: C, 58.0%; H, 6.08%; N, 5.64%. Found: C, 57.85%; H, 6.14%; N, 5.37%

### Example 87 (AD 1285)

### 4-((4-Chloro-N-(2-fluorocyclohexyl)phenylsulfonamido)methyl)-N-(2-(pyrrolidin-1-yl)ethyl)benzamide

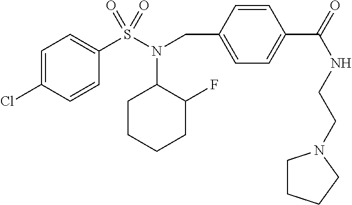 [SMILES: O=C(NCCN1CCCC1)c1ccc(CN(C2CCCCC2F)S(=O)(=O)c2ccc(Cl)cc2)cc1]

4-((4-Chloro-N-(2-fluorocyclohexyl)phenylsulfonamido)methyl)-N-(2-(pyrrolidin-1-yl)ethyl)benzamide (0.092 g, 64.3%) was prepared by the same method as described for Example 60.

MS (m/z): 522.0

Elemental Analysis: C₂₆H₃₃ClFN₃O₃S:

Calcd: C, 59.81%; H, 6.37%; N, 8.05%. Found: C, 59.16%; H, 5.98%; N, 8.61%

### Example 88 (AD 1286)

### 4-((4-Chloro-N-(2-fluorocyclohexyl)phenylsulfonamido)methyl)-N-((R)-1-methoxypropan-2-yl)benzamide

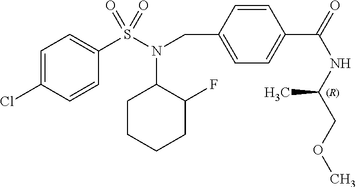

4-((4-Chloro-N-(2-fluorocyclohexyl)phenylsulfonamido)methyl)-N—((R)-1-methoxypropan-2-yl)benzamide (0.092 g, 64.3%) was prepared by the same method as described for Example 60.

MS (m/z): 497.0

Elemental Analysis: C₂₄H₃₀ClFN₂O₄S:

Calcd: C, 58.0%; H, 6.08%; N, 5.64%. Found: C, 57.9%; H, 5.86%; N, 5.54%

### Example 90 (AD 1287)

### 4-((4-Chloro-N-(2-fluorocyclohexyl)phenylsulfonamido)methyl)-N—((S)-1-hydroxypropan-2-yl)benzamide

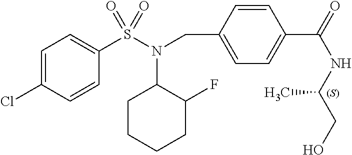

4-((4-Chloro-N-(2-fluorocyclohexyl)phenylsulfonamido)methyl)-N—((S)-1-hydroxypropan-2-yl)benzamide (0.100 g, 59.8%) was prepared by the same method as described for Example 60.

MS (m/z): 483.0

Elemental Analysis: (C₂₃H₂₈ClFN₂O₄S):

Calcd: C, 57.19%; H, 5.84%; N, 5.8%. Found: C, 56.96%; H, 5.6%; N, 5.57%.

### Example 91

This Example describes assays performed to evaluate the biological activity of the compounds described herein.

Cell Lines and Cultures.

HeLa S3 cells, the Chinese hamster ovary (CHO) 7[γ]-30 cell line (co-expressing human PS1, FLAG-Pen-2, and Aph1[alpha]2-HA), and the S-I CHO cell line (co-expressing human PS1, FLAG-Pen-2, Aph1[alpha]2-HA, and NCT-GST) were cultured using reported methods. (See Fraering, P. C, Ye, W., Strub, J. M., Dolios, G., LaVoie, M. J., Ostaszewski, B. L., Van Dorsselaer, A., Wang, R., Selkoe, D. J., and Wolfe, M. S. (2004) Biochemistry 43, 9774-9789; Kimberly, W. T., Esler, W. P., Ye, W., Ostaszewski, B. L., Gao, J., Diehl, T., Selkoe, D. J., and Wolfe, M. S. (2003) Biochemistry 42, 137-144; Fraering, P. C, LaVoie, M. J., Ye, W., Ostaszewski, B. L., Kimberly, W. T., Selkoe, D. J., and Wolfe, M. S. (2004) Biochemistry 43, 323-333.

Purification of γ-Secretase and In Vitro γ-Secretase Assays.

The following procedures were used to isolate γ-secretase and measure its enzymatic activity. The multistep procedure for the high grade purification of human γ-secretase from the S-I cells uses reported methods (Fraering, P. C, et al. (2004) Biochemistry 43, 9774-9789). In vitro γ-secretase assays using the recombinant APP-based substrate C-100 FLAG and the recombinant Notch-based substrate N-100 FLAG have also been reported (Esler, W. P., Kimberly, W. T., Ostaszewski, B. L., Ye, W., Diehl, T. S., Selkoe, D. J., and Wolfe, M. S. (2002) Proc. Natl. Acad. Sci. U.S.A. 99, 2720-2725, Kimberly, W. T., et al. (2003) Biochemistry 42, 137-144). Basically, the proteolytic reaction mixtures contained C-100 FLAG and N-100 FLAG substrates at a concentration of 1 [μM], purified γ-secretase solubilized in 0.2% CHAPSO/HEPES, pH 7.5, at 10-fold dilution from stock (stock=the M2 anti-FLAG-eluted fraction in the purification protocol from S-I cells (Fraering, P. C, et al. (2004) Biochemistry 43, 9774-9789)), 0.025% phosphatidylethanolamine (PE) and 0.10% phosphatidylcholine (PC). All the reactions were stopped by adding 0.5% SDS, and the samples were assayed for Aβ 40 and Aβ 42 by ELISA (Xia, W., Zhang, J., Ostaszewski, B. L., Kimberly, W. T., Seubert, P., Koo, E. H., Shen, J., and Selkoe, D. J. (1998) Biochemistry 31, 16465-16471). The capture antibodies are 2G3 (to Aβ residues 33-40) for the Aβ 40 species and 21F12 (to Aβ residues 33-42) for the Aβ 42 species.

Western Blotting and Antibodies.

The following assay was used to determine the extent to which the compounds of interest modulate the cleavage of APP and the Notch receptor. For Western analysis of PS1-NTF, PS1-CTF, Aph1-α2-HA, FLAG-Pen-2, and NCT-GST, the samples were run on 4-20% Tris-glycine polyacrylamide gels, transferred to polyvinylidene difluoride, and can be probed with Aβ14 (for PS1-NTF, 1:2000; a gift of S. Gandy), 13A11 (for PS1-CTF, 5 μg/mL; a gift of Elan Pharmaceuticals), 3F10 (for Aph1α2-HA, 50 ng/mL; Roche Applied Science), anti-FLAG M2 (for FLAG-Pen-2, 1:1000; Sigma), or α-GST antibodies (for NCT-GST, 1:3000; Sigma). Samples from the γ-secretase activity assays (above) were run on 4-20% Tris-glycine gels and can be transferred to polyvinylidene difluoride membranes to detect AICD-FLAG with anti-FLAG M2 antibodies (1:1000, Sigma) and NICD-FLAG with Notch Aβ 1744 antibody (1:1000, Cell Signaling Technology), which is selective for the N terminus of NICD; the same samples were transferred to nitrocellulose membranes to detect Aβ with the anti-Aβ 6E10 antibody. Levels of AICD-FLAG and NICD-FLAG were estimated by densitometry using AlphaEase/Spot Denso (Alpha Innotech Corp.).

Purified γ-Secretase and Binding to ATP-Immobilized Resins.

The following assay can be used to determine the extent to which the compounds of interest bind to ATP. The purified [gamma]-secretase is diluted 10-fold from stock (Fraering, P. C, et al. (2004) Biochemistry 43, 9774-9789) in 50 mM HEPES buffer, pH 7.0, containing 0.2 or 1% CHAPSO, 150 mM NaCl, 5 mM MgCl₂, 5 mM CaCb and can be incubated overnight, in the presence or absence of 50 mM ATP (Sigma), with ATP-agarose (ATP attached to agarose through the ribose hydroxyls, Sigma catalog number A-4793) or ATP-acrylamide (ATP attached to acrylamide through the γ-phosphate; Novagen catalog number 71438-3). Each resin is washed three times with 0.2 or 1% CHAPSO/HEPES buffer, and the bound proteins are collected in 2× Laemmli sample buffer, and can be resolved on 4-20% Tris-glycine gels, and then transferred to polyvinylidene difluoride membranes to detect NCT-GST, PS1-NTF3 Aph1-HA, PS1-CTF, and FLAG-Pen2 as described above.

Photoaffinity Labeling Experiments.

The following assay can be used to determine the extent to which the compounds of interest inhibit the cleavage of APP. 8-Azido-[γ-³²P]ATP (18 Ci/mmol) is purchased from Affinity Labeling Technology (Lexington, Ky.). For the photoaffinity labeling of the purified γ-secretase, the enzyme is diluted 10-fold from stock (Fraering, P. C, et al. (2004) Biochemistry 43, 9774-9789) in 50 mM HEPES buffer, pH 7.0, containing 0.2% CHAPSO, 150 mM NaCl, 5 mM MgCl₂, 5 mM CaCl₂, 0.025% PE, and 0.10% PC. The samples are exposed to UV light for 5 min (hand-held UV lamp at 254 nm; UVP model UVGL-25) on ice, and the reaction is quenched with 1 mM dithiothreitol. The proteins are diluted in 0.5% CHAPSO/HEPES buffer and incubated overnight for affinity precipitation with GSH resin as described previously (Fraering, P. C, et al. (2004) Biochemistry 43, 9774-9789, Fraering, P. C, et al. (2004) Biochemistry 43, 323-333). The unbound nucleotides are removed by washing the resin three times and then the washed proteins are resuspended in Laemmli sample buffer. For the photoaffinity labeling of the purified [gamma]-secretase followed by the BN-PAGE analysis, the enzyme is diluted in 0.1% digitonin/TBS, exposed to UV light for 5 min, and directly loaded onto a 5-13.5% BN-polyacrylamide gel. For the photoaffinity labeling of endogenous γ-secretase, HeLa S3 membranes (the equivalent of 3.0×10⁸ cells) are incubated with 22.5 μM 8-azido-[γ- ³²P]ATP (10 μCiper reaction), 50 mM HEPES, pH 7.0, 150 mMNaCl, 5 mM MgCl₂, and 5 mMCaCl₂ in a total volume of 60 μL for 10 min at 37° C. The resuspended membranes are exposed to UV light as described above. The unbound nucleotides are removed by washing the membranes three times and then the washed membranes are resuspended for 1 h in 0.5 ml of 1% CHAPSO/HEPES, pH 7.4. The solubilized proteins are diluted 1:2 in HEPES buffer (final CHAPSO concentration=0.5%) and incubated overnight with X81 antibody for immunoprecipitation, as described previously (Fraering, P. C, et al. (2004) Biochemistry 43, 9774-9789, Fraering, P. C, et al. (2004) Biochemistry 43, 323-333). Samples are electrophoresed on 4-20% Tris-glycine gels and autoradiographed (BioMax MS films used with BioMax Transcreen HE (Eastman Kodak Co.)).

ATPase Assays.

The following assay can be used to determine if the compounds of interest compete with ATP. [(X-³²P]ATP (11.9 Ci/mmol) is purchased from Affinity Labeling Technology (Lexington, Ky.). The purified γ-secretase is prepared as described for the photoaffinity labeling experiments; 5 μCi of [(X-³²P]ATP was added; the reactions are incubated at 37° C., and at the indicated time points aliquots are removed and reactions stopped by addition of 10% SDS. A total of 2 μL of each stopped reaction is analyzed by TLC onpolyethyleneimine cellulose plastic sheets (Baker-Flex, Germany) with 0.75 M KH₂PO₄, pH 3.5, as the running buffer to separate ATP from ADP. To identify hydrolysis products, a reaction of [α-³²P]ATP can be incubated in the presence of 0.005 units of canine kidney phosphatase (Sigma). Samples are autoradiographed as described above.

Aβ (1-42) Cellular Assay.

The following assay was used to determine the extent to which the compounds of interest inhibit the cleavage of APP in vivo. AβELISA is a commercial fluorometric kit from Biosource (Invitrogen 89344). Luciferase reporter HEK AP-GL-T1 6 cells were plated at 50,000 cells/well in 96 well plates in DMEM media containing 10% tetracyclin free BSA, 250 μg/mL zeocin, 200 μg/mL hygromycin, and 5 μg/mL blasticidin. Compounds were added 24 h after plating and APP processing is induced simultaneously by addition of tetracycline. Following a 24 h compound treatment, 50 mL of conditioned cell media is collected, mixed with ELISA diluent buffer containing 2 mM AEBSF and 12 mM o-phenanthroline, and immediately frozen at −80° C. For the ELISA, the samples were brought to room temperature and spun at 5000 rpm for 5 min. Samples (50 mL) are incubated in the ELISA plate with 50 mL detection antibody on a shaker at room temperature for 3 h. Wells were then washed 4 times with wash buffer and 100 mL of secondary antibody are added and incubated at room temperature for 30 min. Wells were again washed 4 times with wash buffer and 100 mL of fluorescent substrate solution are added. After 30 minutes of incubation, fluorescent signals are determine on a Gemini reader at ex 460 nm and em 560 nm. The amount of Aβ levels in each sample was determined from a standard curve generated by known concentrations of Aβ peptide run simultaneously with the samples.

EC₅₀ Determination with Tetracycline.

Cells are trypsinized using trypsin-EDTA (Invitrogen) and harvested by centrifugation at 151 Og. The pellet is then resuspended with DMEM-HZB. The density of cells is determined with a hematocytometer, and cells (500 cells μL) are transferred at 40 μL/well into 384-well Nunc cell culture plates. Cells are incubated at 37° C. in a CO₂ incubator for 24 h. Serially diluted tetracycline is added to media starting from a 5 μg/mL concentration on a separate plate. For each concentration, 10 wells are used. For negative control, no tetracycline is added to media. On the second day, 10 [μL/well of media with/without tetracycline is added. After an additional 48 h of incubation, the plates are brought to room temperature, and 50 μL of luciferase substrate is added. The luminescence is then read using an LJL Analyst (Molecular Device).

IC₅₀ Determination of a γ-Secretase Inhibitor.

The following assay can be used to determine the concentration of a compound of the invention required to achieve 50% inhibition of γ-secretase activity. Serial 3-fold dilutions of compound E, a potent inhibitor of γ-secretase, starting at 3 μM final concentration, are prepared on a separate plate using media with tetracycline, and 10 μL of each is added to 384-well Nunc white plates containing cells (final concentration of tetracycline is 1 μg/mL) Ten replicates are used for each concentration, and the experiment is performed 3 times. The plates are further incubated for 48 h after tetracycline addition. After bringing the temperature down to room temperature, 50 μL of luciferase substrate/well is added and mixed, and luminescence is recorded with an LJL Analyst (Molecular Device).

MTS Assay.

The following assay was used to indicate the number of viable cells in proliferation and thereby evaluate the toxicity of a candidate compound. The MTS assay used is Promega's Cell Titer 96 Aqueous One Solution Cell Proliferation Assay. It is a colorometric assay that indicates the number of viable cells in proliferation by measuring the amount of MTS reduced to formazan by NADPH or NADH produced by metabolically active cells. After conditioned media was collected for the ELISA, MTS reagent is added to sample at a ratio of 20 mL reagent to 100 mL cell media. Samples were incubated for 1 h at 37° C. in a 5% CO₂ incubator. Then absorbance was recorded at 490 run with a Gemini reader. Cell viability was assessed by determining the percent sample signal to untreated controls. All sample and control signals were adjusted to a background signal determined from cells lysed with 0.9% Triton X.

Notch Cellular Assay.

This assay was used to determine if the compounds of interest inhibit the cleavage of Notch by γ-secretase in cells. A U2OS cell line in which the luciferase expression is adjusted by active Notch was used in this assay Notch expression is adjusted using Tet-on promoter. Luciferase reporter U2OS cells are plated at 1000 cells/well in 96 well plates in DMEM containing 100 μg/mL hygromycin, 15 μg/mL blasticidin and 1 μg/mL Tetracycline. Compounds were added 24 h after plating and the cells are lysed 6 days after adding compounds. Luciferase expression was performed using Steady-Glo Luciferase Assay System (Promega).

Cleaved Notch1 Sandwich ELISA.

This assay was used to determine if the compounds of interest inhibit the Notch cleavage activity of γ-secretase in SUP-T1 cell line, which endogenously expresses a truncated Notch receptor fragment that is a viable gamma-secretase substrate. The base medium for this cell line is ATCC-formulated RPMI-1640 Medium with fetal bovine serum to a final concentration of 10%. Prepare dilution of SUP-T1 cells in complete growth medium to 1.0×10⁶ cells/mL, seed 1.5 mL per well of each cell dilution into a 6-well plate. Collect cells treated with serial diluted compounds in DMSO after 16 h, then using equal amount of protein isolated from samples to measure Notch intracellular cleavage by PathScan® Sandwich ELISA Kit (Cell Signaling Tech 7194S).

| | | Aβ42 | Notch | | Notch | |
|---|---|---|---|---|---|---|
| | | % Inh [μM] | Luciferase | Notch | Elisa | Notch |
| | | or IC50 value | IC50 value | Luc/ | IC50 value | Elisa/ |
| EX/AD | STRUCTURE | (μM) | (μM) | Aβ42 | (μM) | Aβ42 |
| 1/965 | 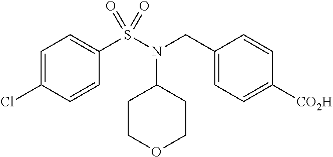 [SMILES: O=C(O)c1ccc(CN(C2CCOCC2)S(=O)(=O)c2ccc(Cl)cc2)cc1] | 6[1] | | | | |
| 2/1094 | 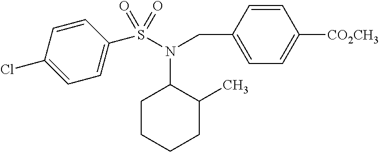 [SMILES: COC(=O)c1ccc(CN(C2CCCCC2C)S(=O)(=O)c2ccc(Cl)cc2)cc1] | 0.0034 | 3 | 882 | | |
| 3/1112 | 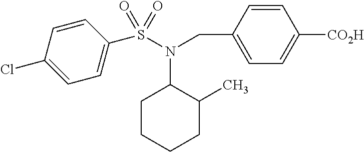 [SMILES: CC1CCCCC1N(Cc1ccc(C(=O)O)cc1)S(=O)(=O)c1ccc(Cl)cc1] | 0.007, 0.0127, 0.0038 | 5, 4.4, 4.3 | 585 | 0.35 | 45 |
| 4/1113 | 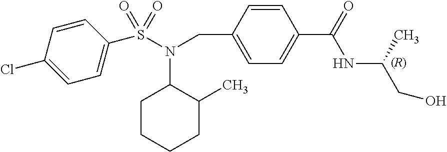 | 0.00087, 0.00065, 0.00089 | 1, 283, 1.075 | 2950 | 0.18, 0.12 | 188 |
| 5/1138 | 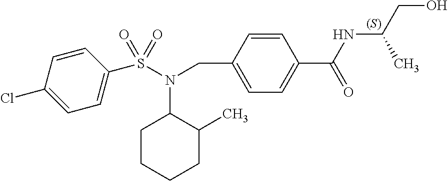 | 0.0011, 0.0018 | 2.3 | 1586 | 0.16 | 110 |
| 6/1139 | 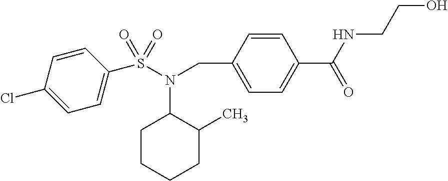 [SMILES: CC1CCCCC1N(Cc1ccc(C(=O)NCCO)cc1)S(=O)(=O)c1ccc(Cl)cc1] | 0.00083 | 0.99 | 1192 | 0.13 | 157 |
| 7/1143 | 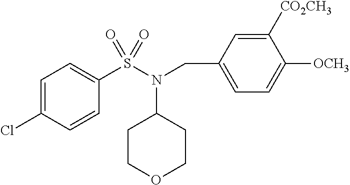 [SMILES: COC(=O)c1cc(CN(C2CCOCC2)S(=O)(=O)c2ccc(Cl)cc2)ccc1OC] | 0[1] | | | | |
| 8/1144 | 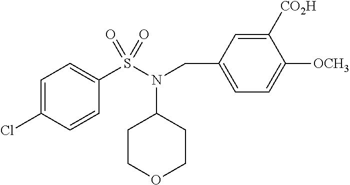 [SMILES: COc1ccc(CN(C2CCOCC2)S(=O)(=O)c2ccc(Cl)cc2)cc1C(=O)O] | 0[1] | | | | |
| 9/1145 | 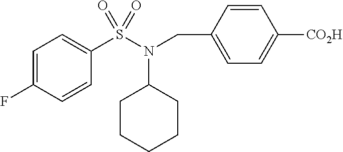 [SMILES: O=C(O)c1ccc(CN(C2CCCCC2)S(=O)(=O)c2ccc(F)cc2)cc1] | 0[1] | | | | |
| 10/953 | 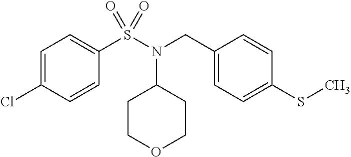 [SMILES: CSc1ccc(CN(C2CCOCC2)S(=O)(=O)c2ccc(Cl)cc2)cc1] | 0[1] | | | | |
| 11/954 | 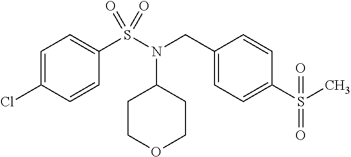 [SMILES: CS(=O)(=O)c1ccc(CN(C2CCOCC2)S(=O)(=O)c2ccc(Cl)cc2)cc1] | 0[1] | | | | |
| 12/955 | 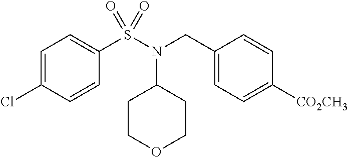 [SMILES: COC(=O)c1ccc(CN(C2CCOCC2)S(=O)(=O)c2ccc(Cl)cc2)cc1] | 0[1] | | | | |
| 13/956 | 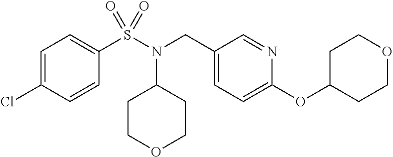 [SMILES: O=S(=O)(c1ccc(Cl)cc1)N(Cc1ccc(OC2CCOCC2)nc1)C1CCOCC1] | 0[1] | | | | |
| 14/1136 = 46/1221 | 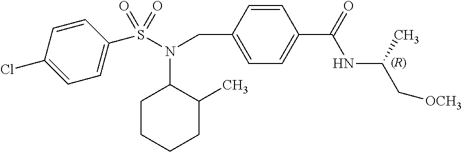 | 0.0007 | 0.303 | 433 | 0.71 | 1014 |
| 15/1155 | 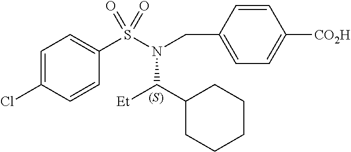 | 0.028 | 20 | 715 | Pending | |
| 16/1159 | 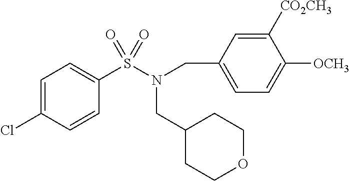 [SMILES: COC(=O)c1cc(CN(CC2CCOCC2)S(=O)(=O)c2ccc(Cl)cc2)ccc1OC] | 0[1] | | | | |
| 17/1161 | 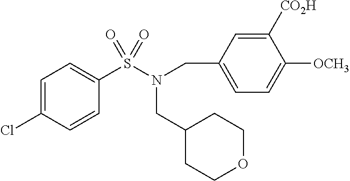 [SMILES: COc1ccc(CN(CC2CCOCC2)S(=O)(=O)c2ccc(Cl)cc2)cc1C(=O)O] | 14[1] | | | | |
| 18/1162 | 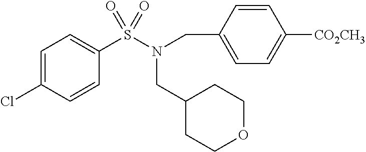 [SMILES: COC(=O)c1ccc(CN(CC2CCOCC2)S(=O)(=O)c2ccc(Cl)cc2)cc1] | 94[1] | | | | |
| 19/1163 | 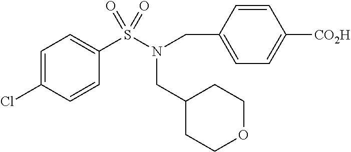 [SMILES: O=C(O)c1ccc(CN(CC2CCOCC2)S(=O)(=O)c2ccc(Cl)cc2)cc1] | 13[1] | | | | |
| 20/1164 | 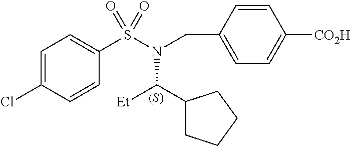 | 0.05 | 40, 100 | 1400 | Pending | |
| 21/1165 | 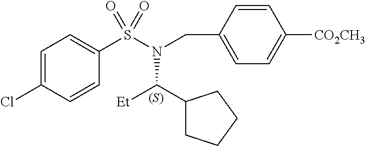 | 0.024 | 13 | 542 | | |
| 22/1166 | 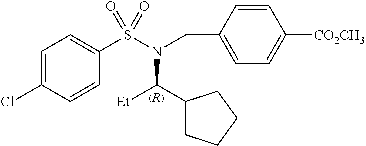 | 0[1] | | | | |
| 23/1167 | 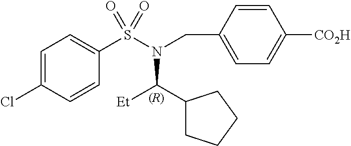 | 0[1] | | | | |
| 24/1188 | 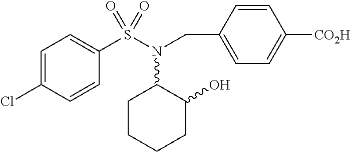 [SMILES: O=C(O)c1ccc(CN(C2CCCCC2O)S(=O)(=O)c2ccc(Cl)cc2)cc1] | 51[1] | | | | |
| 25/1189 | 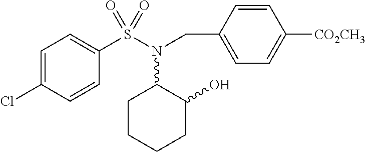 [SMILES: COC(=O)c1ccc(CN(C2CCCCC2O)S(=O)(=O)c2ccc(Cl)cc2)cc1] | 0.01781 | | | | |
| 26/1178 | 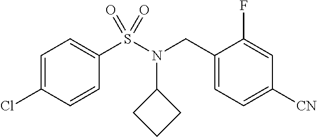 [SMILES: N#Cc1ccc(CN(C2CCC2)S(=O)(=O)c2ccc(Cl)cc2)c(F)c1] | 54[1] | | | | |
| 27/1179 | 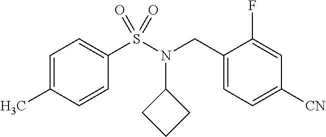 [SMILES: Cc1ccc(S(=O)(=O)N(Cc2ccc(C#N)cc2F)C2CCC2)cc1] | 37[1] | | | | |
| 28/1180 | 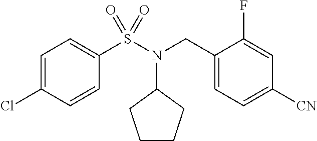 [SMILES: N#Cc1ccc(CN(C2CCCC2)S(=O)(=O)c2ccc(Cl)cc2)c(F)c1] | 0.301 | | | Pending | |
| 29/1181 | 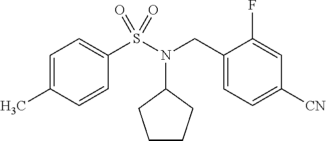 [SMILES: Cc1ccc(S(=O)(=O)N(Cc2ccc(C#N)cc2F)C2CCCC2)cc1] | 0[1] | | | | |
| 30/1192 | 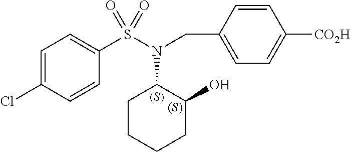 | 15[1] | | | | |
| 31/1193 | 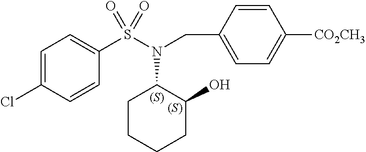 | 0.1985 | | | | |
| 32/1194 | 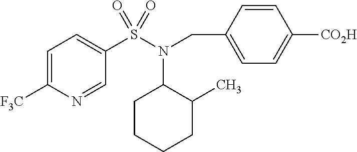 [SMILES: CC1CCCCC1N(Cc1ccc(C(=O)O)cc1)S(=O)(=O)c1ccc(C(F)(F)F)nc1] | 0.02844 | 4.8 | 169 | | |
| 33/1196 | 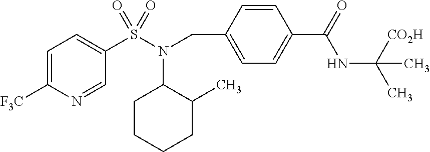 [SMILES: CC1CCCCC1N(Cc1ccc(C(=O)NC(C)(C)C(=O)O)cc1)S(=O)(=O)c1ccc(C(F)(F)F)nc1] | 56[1] | | | | |
| 34/1198 | 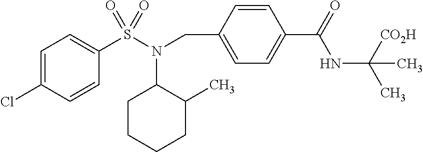 [SMILES: CC1CCCCC1N(Cc1ccc(C(=O)NC(C)(C)C(=O)O)cc1)S(=O)(=O)c1ccc(Cl)cc1] | 0.135 | 8.089 | 60 | | |
| 35/1201 | 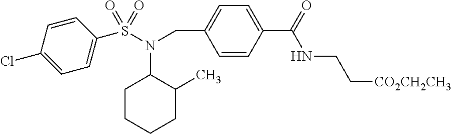 | 0.0297 | | | | |
| 36/1202 | 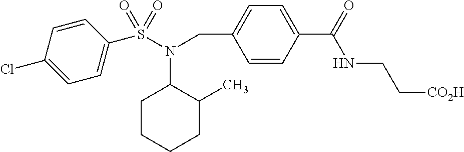 [SMILES: CC1CCCCC1N(Cc1ccc(C(=O)NCCC(=O)O)cc1)S(=O)(=O)c1ccc(Cl)cc1] | 0.08071 | 4.579 | 57 | | |
| 37/1204 | 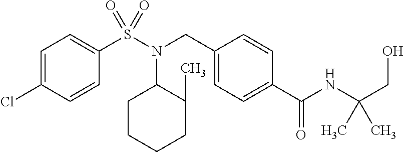 [SMILES: CC1CCCCC1N(Cc1ccc(C(=O)NC(C)(C)CO)cc1)S(=O)(=O)c1ccc(Cl)cc1] | 0.02087 | 2.536 | 122 | | |
| 38/1206 | 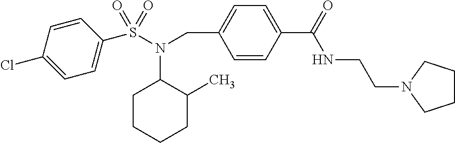 [SMILES: CC1CCCCC1N(Cc1ccc(C(=O)NCCN2CCCC2)cc1)S(=O)(=O)c1ccc(Cl)cc1] | 0.008303, 0.01279 | | | 0.667 | 64 |
| 39/1207 | 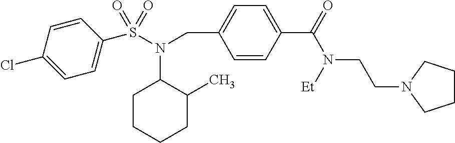 [SMILES: CCN(CCN1CCCC1)C(=O)c1ccc(CN(C2CCCCC2C)S(=O)(=O)c2ccc(Cl)cc2)cc1] | 0.04067, 0.07744 | | | 2.262 | 38 |
| 40/1208 | 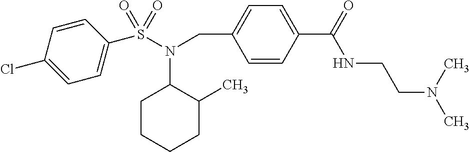 [SMILES: CC1CCCCC1N(Cc1ccc(C(=O)NCCN(C)C)cc1)S(=O)(=O)c1ccc(Cl)cc1] | 0.002562 | 3.031 | 118 | | |
| 41/1209-2 | 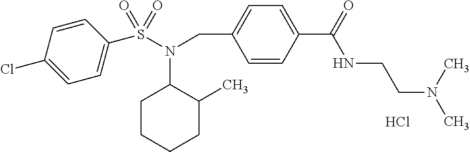 [SMILES: CC1CCCCC1N(Cc1ccc(C(=O)NCCN(C)C)cc1)S(=O)(=O)c1ccc(Cl)cc1 ; Cl] | 0.0058, 0.0109 | | | 0.6754, 0.850 | 91 |
| 42/1211 | 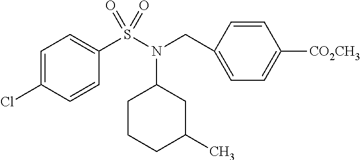 [SMILES: COC(=O)c1ccc(CN(C2CCCC(C)C2)S(=O)(=O)c2ccc(Cl)cc2)cc1] | 0.009443 | | | | |
| 43/1213 | 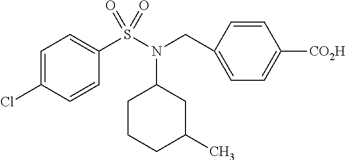 [SMILES: CC1CCCC(N(Cc2ccc(C(=O)O)cc2)S(=O)(=O)c2ccc(Cl)cc2)C1] | 0.148 | | | 2.821, 2.73 | 19 |
| 44/1212 | 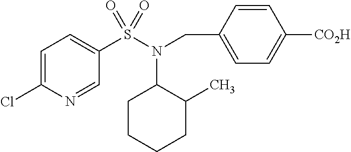 [SMILES: CC1CCCCC1N(Cc1ccc(C(=O)O)cc1)S(=O)(=O)c1ccc(Cl)nc1] | 0.006403, 0.02002 | | | 2.98, 2.73 | 220 |
| 45/1216 | 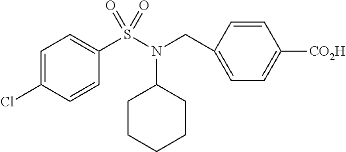 [SMILES: O=C(O)c1ccc(CN(C2CCCCC2)S(=O)(=O)c2ccc(Cl)cc2)cc1] | 0.216, 0.0474, 0.180 | | | 35.94 | 243 |
| 46/1221 | 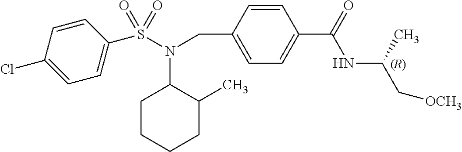 | 0.0006997 | 0.3034 | 432 | 0.71 | 1014 |
| 47/1222 | 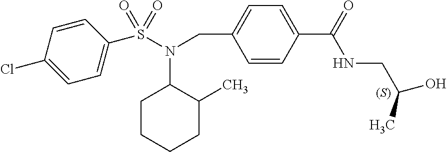 | 0.0008725 | 0.6901 | 791 | 0.35 | 401 |
| 48/1223 | 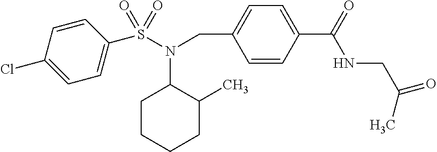 [SMILES: CC(=O)CNC(=O)c1ccc(CN(C2CCCCC2C)S(=O)(=O)c2ccc(Cl)cc2)cc1] | 0.0007147 | 0.1817 | 260 | 0.22 | 314 |
| 49/1225 | 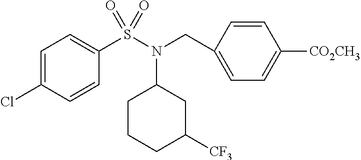 [SMILES: COC(=O)c1ccc(CN(C2CCCC(C(F)(F)F)C2)S(=O)(=O)c2ccc(Cl)cc2)cc1] | 0.007304 | | | | |
| 50/1226 | 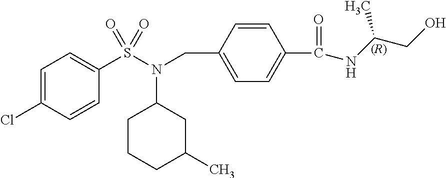 | 0.007324 | 10.63 | 1456 | 2.16 | 309 |
| 51/1227 | 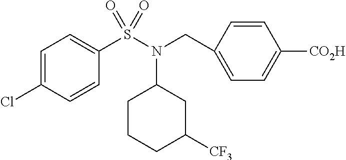 [SMILES: O=C(O)c1ccc(CN(C2CCCC(C(F)(F)F)C2)S(=O)(=O)c2ccc(Cl)cc2)cc1] | 0.06216, 06994 | 3.226 | 52 | | |
| 52/1227C | 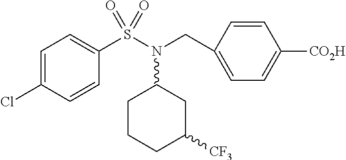 [SMILES: O=C(O)c1ccc(CN(C2CCCC(C(F)(F)F)C2)S(=O)(=O)c2ccc(Cl)cc2)cc1] | 0.04759 | | | 1.88 | 40 |
| 53/1227D | 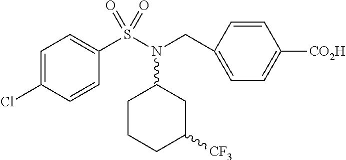 [SMILES: O=C(O)c1ccc(CN(C2CCCC(C(F)(F)F)C2)S(=O)(=O)c2ccc(Cl)cc2)cc1] | 1.185 | | | 34.79 | 29 |
| 54/1228 | 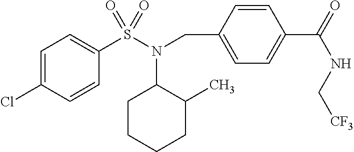 [SMILES: CC1CCCCC1N(Cc1ccc(C(=O)NCC(F)(F)F)cc1)S(=O)(=O)c1ccc(Cl)cc1] | 0.002614 | 0.5744 | 220 | | |
| 55/1229 | 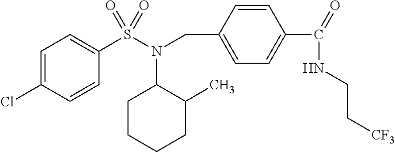 [SMILES: CC1CCCCC1N(Cc1ccc(C(=O)NCCC(F)(F)F)cc1)S(=O)(=O)c1ccc(Cl)cc1] | 0.004298 | 1.1 | 256 | | |
| 56/1230 | 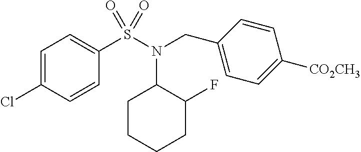 [SMILES: COC(=O)c1ccc(CN(C2CCCCC2F)S(=O)(=O)c2ccc(Cl)cc2)cc1] | 0.004051 | | | | |
| 57/1231 | 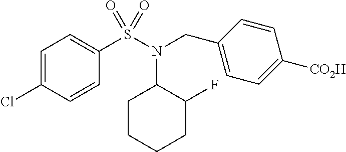 [SMILES: O=C(O)c1ccc(CN(C2CCCCC2F)S(=O)(=O)c2ccc(Cl)cc2)cc1] | 0.0501, 0.0796, 0.0985 | 28.61 | 376 | 1.07 | 14 |
| 58/1231A | 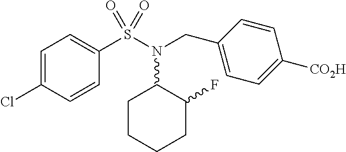 [SMILES: O=C(O)c1ccc(CN(C2CCCCC2F)S(=O)(=O)c2ccc(Cl)cc2)cc1] | 0.341, 0.686, 0.207 | 259 | 630 | 10.07 | 25 |
| 59/1231B | 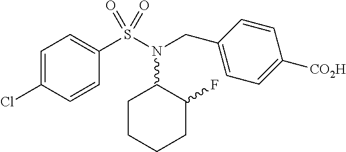 [SMILES: O=C(O)c1ccc(CN(C2CCCCC2F)S(=O)(=O)c2ccc(Cl)cc2)cc1] | 0.0607, 0.195, 0.101 | 9.4 | 79 | 2.93 | 25 |
| 60/1235 | 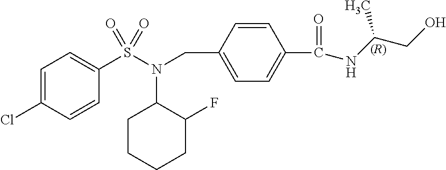 | 0.00458, 0.007 | | | 1.555 | 305 |
| 61/1240 | 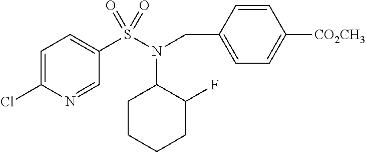 [SMILES: COC(=O)c1ccc(CN(C2CCCCC2F)S(=O)(=O)c2ccc(Cl)nc2)cc1] | 0.01036 | | | | |
| 62/1241 | 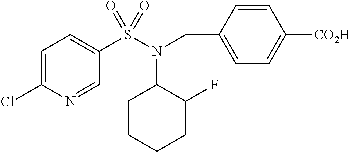 [SMILES: O=C(O)c1ccc(CN(C2CCCCC2F)S(=O)(=O)c2ccc(Cl)nc2)cc1] | 0.135, 0.261 | | | 5.2 | 26 |
| 63/1242 | 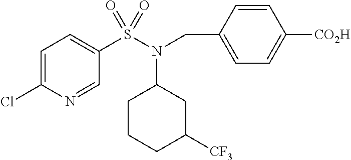 [SMILES: O=C(O)c1ccc(CN(C2CCCC(C(F)(F)F)C2)S(=O)(=O)c2ccc(Cl)nc2)cc1] | 87[1] | | | | |
| 64/1243 | 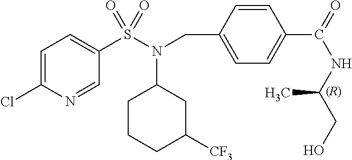 | 0.01201 | | | 1.07 | 89 |
| 65/1244 | 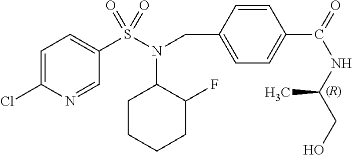 | 0.01375 | | | 1.15 | 84 |
| 66/1245 | 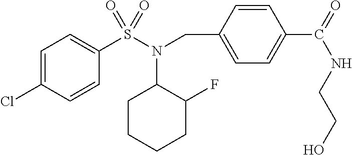 [SMILES: O=C(NCCO)c1ccc(CN(C2CCCCC2F)S(=O)(=O)c2ccc(Cl)cc2)cc1] | 0.01027 | | | 1.38 | 134 |
| 67/1249 | 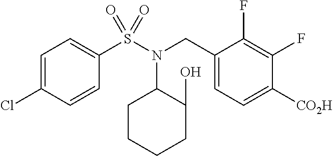 [SMILES: O=C(O)c1ccc(CN(C2CCCCC2O)S(=O)(=O)c2ccc(Cl)cc2)c(F)c1F] | 11[1] | | | | |
| 68/1250 | 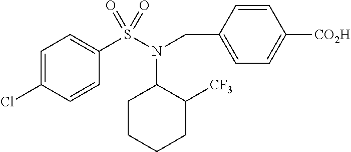 [SMILES: O=C(O)c1ccc(CN(C2CCCCC2C(F)(F)F)S(=O)(=O)c2ccc(Cl)cc2)cc1] | 0.02553 | | | 0.83 | 33 |
| 69/1251 | 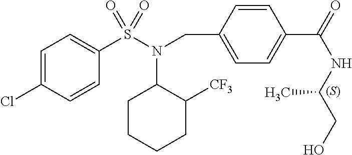 | 0.002164 | | | 0.094 | 44 |
| 70/1252 | 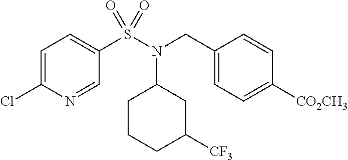 [SMILES: COC(=O)c1ccc(CN(C2CCCC(C(F)(F)F)C2)S(=O)(=O)c2ccc(Cl)nc2)cc1] | 0.02683 | | | | |
| 71/1253 | 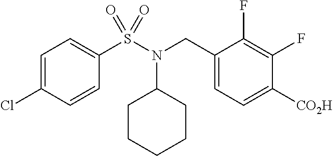 [SMILES: O=C(O)c1ccc(CN(C2CCCCC2)S(=O)(=O)c2ccc(Cl)cc2)c(F)c1F] | 0.5193 | | | | |
| 72/1254 | 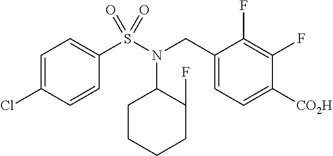 [SMILES: O=C(O)c1ccc(CN(C2CCCCC2F)S(=O)(=O)c2ccc(Cl)cc2)c(F)c1F] | 0.04848 | | | 3.54 | 73 |
| 73/1255 | 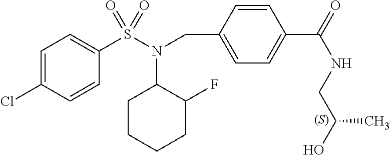 | 0.01038 | | | 1.10 | 106 |
| 74/1257 | 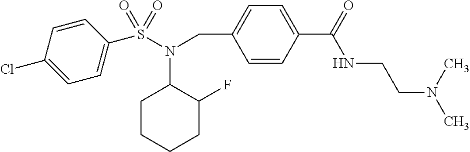 [SMILES: CN(C)CCNC(=O)c1ccc(CN(C2CCCCC2F)S(=O)(=O)c2ccc(Cl)cc2)cc1] | 0.1295 | | | 10.04 | 78 |
| 75/1257S | 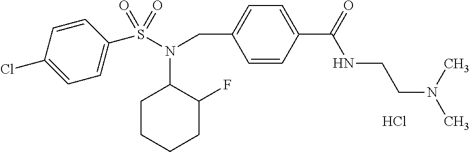 [SMILES: CN(C)CCNC(=O)c1ccc(CN(C2CCCCC2F)S(=O)(=O)c2ccc(Cl)cc2)cc1 ; Cl] | 0.0328, 0.0707 | | | 4.137 | 80 |
| 76/1258 | 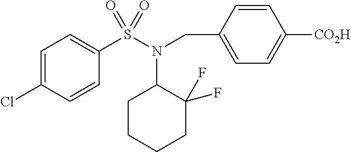 [SMILES: O=C(O)c1ccc(CN(C2CCCCC2(F)F)S(=O)(=O)c2ccc(Cl)cc2)cc1] | 0.08988 | | | 5.06 | 56 |
| 77/1259 | 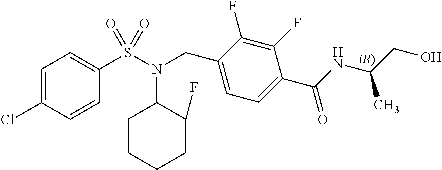 | 0.00424, 0.00336 | | | 1.02 | 268 |
| 78/1264 | 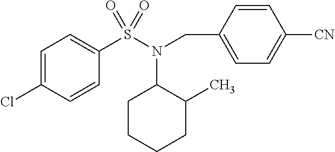 [SMILES: CC1CCCCC1N(Cc1ccc(C#N)cc1)S(=O)(=O)c1ccc(Cl)cc1] | 0.01403 | | | 0.879 | 63 |
| 79/1265 | 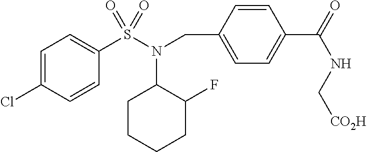 [SMILES: O=C(O)CNC(=O)c1ccc(CN(C2CCCCC2F)S(=O)(=O)c2ccc(Cl)cc2)cc1] | 3.616 | | | | |
| 80/1275S | 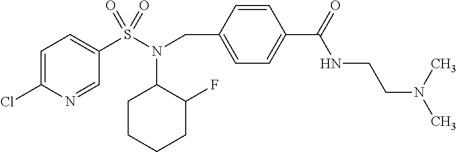 [SMILES: CN(C)CCNC(=O)c1ccc(CN(C2CCCCC2F)S(=O)(=O)c2ccc(Cl)nc2)cc1] | 65[1] | | | | |
| 81/1276 | 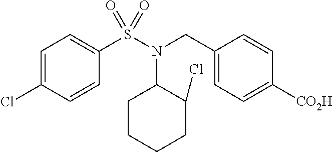 [SMILES: O=C(O)c1ccc(CN(C2CCCCC2Cl)S(=O)(=O)c2ccc(Cl)cc2)cc1] | 0[30] | | | | |
| 82/1277 | 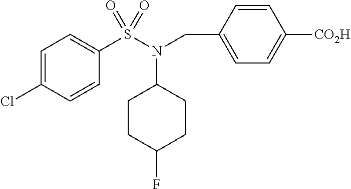 [SMILES: O=C(O)c1ccc(CN(C2CCC(F)CC2)S(=O)(=O)c2ccc(Cl)cc2)cc1] | 0[30] | | | | |
| 83/1278 | 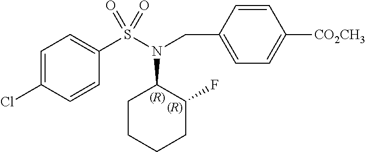 | 0.04429 | | | 1.57 | 35 |
| 84/1279 | 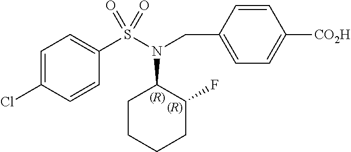 | 0.1408 | | | Pending | |
| 85/1280 | 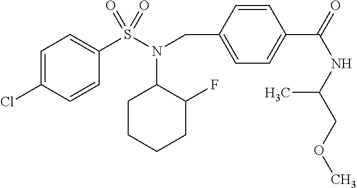 [SMILES: COCC(C)NC(=O)c1ccc(CN(C2CCCCC2F)S(=O)(=O)c2ccc(Cl)cc2)cc1] | 0.01328 | | | 1.183 | 89 |
| 86/1281 | 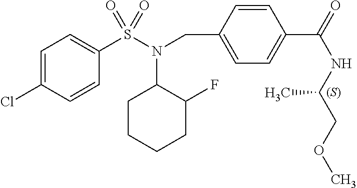 | 0.1031 | | | Pending | |
| 87/1285 | 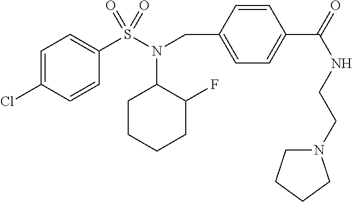 [SMILES: O=C(NCCN1CCCC1)c1ccc(CN(C2CCCCC2F)S(=O)(=O)c2ccc(Cl)cc2)cc1] | 0.00691 | | | Pending | |
| 88/1286 | 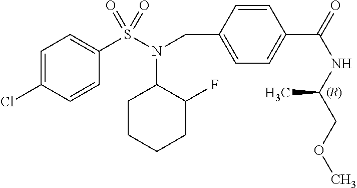 | 0.007309 | | | Pending | |
| 89/1287 | 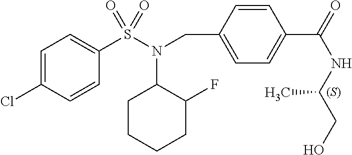 | 0.03537 | | | Pending | |

| | |
|---|---|
| 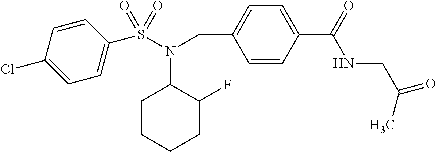 [SMILES: CC(=O)CNC(=O)c1ccc(CN(C2CCCCC2F)S(=O)(=O)c2ccc(Cl)cc2)cc1] | 4-((4-chloro-N-(2- fluorocyclohexyl)phenylsulfonamido)methyl)-N-(2- oxopropyl)benzamide |
| 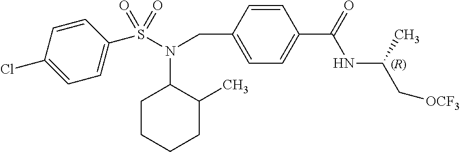 | 4-((4-chloro-N-(2- methylcyclohexyl)phenylsulfonamido)methyl)-N-((R)-1- (trifluoromethoxy)propan-2-yl)benzamide |
| 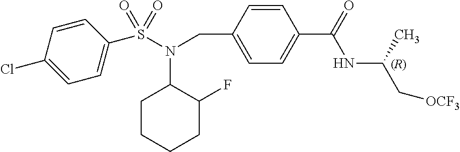 | 4-((4-chloro-N-(2- fluorocyclohexyl)phenylsulfonamido)methyl)-N-((R)-1- (trifluoromethoxy)propan-2-yl)benzamide |
| 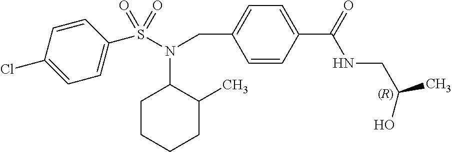 | 4-((4-chloro-N-(2- methylcyclohexyl)phenylsulfonamido)methyl)-N-((R)-2- hydroxypropyl)benzamide |
| 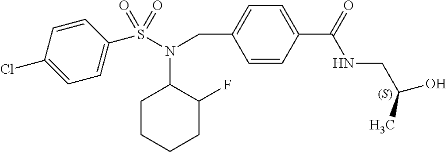 | 4-((4-chloro-N-(2- fluorocyclohexyl)phenylsulfonamido)methyl)-N-((S)-2- hydroxypropyl)benzamide |
| 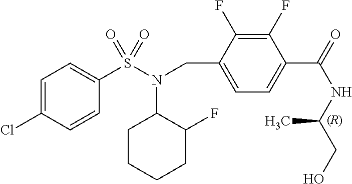 | 4-((4-chloro-N-(2- fluorocyclohexyl)phenylsulfonamido)methyl)-2,3- difluoro-N-((R)-1-hydroxypropan-2-yl)benzamide |
| 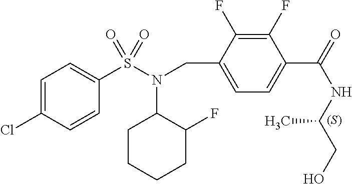 | 4-((4-chloro-N-(2- fluorocyclohexyl)phenylsulfonamido)methyl)-2,3- difluoro-N-((S)-1-hydroxypropan-2-yl)benzamide |
| 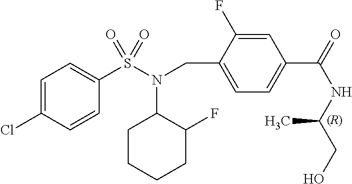 | 4-((4-chloro-N-(2- fluorocyclohexyl)phenylsulfonamido)methyl)-3-fluoro- N-((R)-1-hydroxypropan-2-yl)benzamide |
| 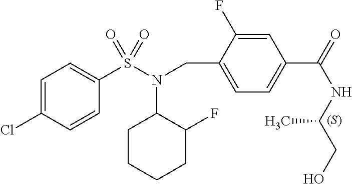 | 4-((4-chloro-N-(2- fluorocyclohexyl)phenylsulfonamido)methyl)-3-fluoro- N-((S)-1-hydroxypropan-2-yl)benzamide |
| 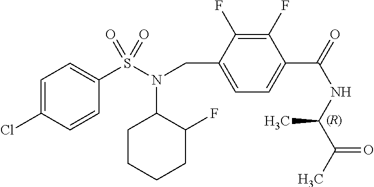 | 4-((4-chloro-N-(2- fluorocyclohexyl)phenylsulfonamido)methyl)-3-fluoro- N-((R)-1-hydroxypropan-2-yl)benzamide |

1. A compound of the formula (I): 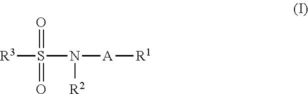
wherein:
R′ is: 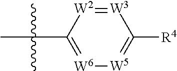
wherein:
W², W³, W⁵, and W⁶ are defined according to (A) or (B) below:
(A)
each of W² and W⁶ is independently selected from CH, C(halo), C(C₁-C₆ alkoxy) and C(C₁-C₆ haloalkoxy); and
each of W³ and W⁵ is independently selected from CH; C(halo); C(C₁-C₆ alkoxy); and CR′; wherein R′ is —C(O)OH, —C(O)OR⁴¹, —C(O)NR⁴²R⁴³; or —CN; or
(B)
one or two of W², W³, W⁵, and W⁶ are N; and the others are independently selected from CH, C(halo), C(C₁-C₆ alkoxy), and C(C₁-C₆ haloalkoxy);
R⁴ is selected from any of the substituents delineated in (i)-(v) immediately below:
(i) halo; —CO₂H; —C(O)OR⁴¹; —NHC(O)OR⁴¹; —N(CH₃)C(O)OR⁴¹;
NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
(iv) heteroaryl containing 5 ring atoms, wherein from 1-4 of the ring atoms is independently selected from N, NH, N(C₁-C₆ alkyl), O, and S; and wherein said heteroaryl is optionally substituted with from 1-3 independently selected R^{b}; or
(v) hydrogen;
R⁴¹ is C₁-C₈ alkyl, C₁-C₈ haloalkyl, or benzyl optionally substituted with from 1-3 R^{b};
each of R⁴² and R⁴³ is, independently:
(i) hydrogen; or
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 R^{c};
or
R⁴²—N—R⁴³ together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{c};
R⁴⁴ is hydrogen, C₁-C₈ alkyl, or C₁-C₈ haloalkyl;
R⁴⁵ is C₁-C₈ alkyl or C₁-C₈ haloalkyl;
in embodiments, it is provided that only one of R⁴ and R′ or only one of R⁴ and two occurrences of R′ can be —C(O)OH, —C(O)O(R⁴¹, e.g., C₁-C₆ alkyl), —C(O)NR⁴²R⁴³; or —CN;
A is C(R^{A})₂, wherein each occurrence of R^{A} is independently selected from hydrogen, fluoro, and —CH₃;
R² is: 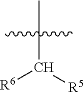
wherein R⁵ and R⁶ are defined according to (C) or (D) below:
(C)
R⁵ and R⁶, together with the carbon atom to which each is attached, is C₃-C₈ cycloalkyl; or heterocyclyl containing from 3-8 ring atoms, wherein from 1-2 of the ring atoms is independently selected from N, NH, N(C₁-C₆ alkyl), O, and S; wherein each of said cycloalkyl and heterocyclyl is optionally substituted with from 1-5 R^{c}; or
(D)
R⁵ is C₃-C₈ cycloalkyl; or heterocyclyl containing from 3-8 ring atoms, wherein from 1-2 of the ring atoms is independently selected from N, NH, N(C₁-C₆ alkyl), O, and S; wherein each of said cycloalkyl and heterocyclyl is optionally substituted with from 1-5 R^{c}; and
R⁶ is C₁-C₆ alkyl or C₁-C₆ haloalkyl, each of which is optionally substituted with a substituent selected from —OH, OCH₃, OCF₃, and —CN;
R³ is:
(i) C₆-C₁₀ aryl, which is optionally substituted with from 1-3 independently selected R^{d};
or
(ii) heteroaryl, each containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d};
R^{a} at each occurrence is, independently, selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN;
R^{b} at each occurrence is, independently selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —NH₂, —NH(C₁-C₆ alkyl), N(C₁-C₆ alkyl)₂, —NHC(O)(C₁-C₆ alkyl), —CN; and —NO₂;
R^{c} at each occurrence is independently selected from the substituents delineated in (aa), (bb) and (cc) below:
(aa) halo; C₁-C₆ alkoxy; C₁-C₆ haloalkoxy; C₁-C₆ thioalkoxy; C₁-C₆ thiohaloalkoxy; C₁-C₆ alkyl, C₁-C₆ haloalkyl, —NH(C₁-C₆ alkyl), N(C₁-C₆ alkyl)₂, —NHC(O)(C₁-C₆ alkyl), wherein the alkyl portion of each is optionally substituted with —OH, C₁-C₃ alkoxy, —C(O)OH, —C(O)O(C₁-C₆ alkyl), and —CN;
(bb) —OH; —CN; —NH₂; C₂-C₄ alkenyl; C₂-C₄ alkynyl; —C(O)H; —C(O)(C₁-C₆ alkyl); C(O)OH; —C(O)O(C₁-C₆ alkyl); —C(O)NH₂; —SO₂(C₁-C₆ alkyl); —SO₂(C₁-C₆ haloalkyl); —C(O)NR″′R″″, —SO₂NR″′R″″, —SO₂NH₂, —NHCO(C₁-C₆ alkyl), —NHSO₂(C₁-C₆ alkyl), whereby R″′ and R″″ is independently selected from H, C₁-C₆ alkyl, C₁-C₆ haloalkyl;
(cc) C₃-C₆ cycloalkyl; or heterocyclyl or heterocyclyloxy, each containing from 5-6 ring atoms, wherein from 1-2 of the ring atoms of the heterocyclyl (or heterocyclyl portion) is independently selected from N, NH, N(C₁-C₆ alkyl), NC(O)(C₁-C₆ alkyl), O, and S; and wherein each of said cycloalkyl, heterocyclyl, and heterocyclyloxy is optionally substituted with from 1-3 substituents independently selected from —OH and C₁-C₄ alkyl;
and
R^{d} at each occurrence is, independently selected from halo, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN; COOH, NO₂, C(O)(C₁-C₆ alkyl), C(O)(C₁-C₆ haloalkyl), azido, NCS, —CH₂OH, amino, NR″′R″″, N-azidinyl, N-morpholinyl, S(C₁-C₆ alkyl), —SO₂(C₁-C₆ alkyl), —C(O)NR″′R″″—SO₂NR″′R″″, —SO₂NH₂, —NHCO(C₁-C₆ alkyl), —NHSO₂(C₁-C₆ alkyl), whereby R″′ and R″″ is independently selected from H, C₁-C₆ alkyl, C₁-C₆ haloalkyl;
or a pharmaceutically acceptable salt thereof.

2. The compound of claim 1, wherein W², W³, W⁵, and W⁶ are defined according to definition (A).

3. The compound of claim 1 or 2, wherein each of W³ and W⁵ is independently selected from CH, C(halo), and C(C₁-C₆ alkoxy).

4. The compound according to any one of claims 1-3, wherein each of W², W³, W⁵, and W⁶ is independently selected from CH and C(halo).

5. The compound according to any one of claims 1-4, wherein each of W², W³, W⁵, and W⁶ is CH.

6. The compound of claim 2, wherein one of W³ and W⁵ is CR′, and the other of W³ and W⁵ is CH, C(halo), or C(C₁-C₆ alkoxy).

7. The compound of claim 6, wherein one of W³ and W⁵ is CR′, and the other of W³ and W⁵ is CH.

8. The compound of claim 6 or 7, wherein each of W² and W⁶ is independently selected from CH and C(halo).

9. The compound according to any one of claims 6-8, wherein R′ is —C(O)OH or —C(O)O(C₁-C₆ alkyl).

10. The compound of claim 1, wherein W², W³, W⁵, and W⁶ are defined according to definition (B).

11. The compound of claim 10, wherein one or two of W³ and W⁵ is/are N.

12. The compound of claim 10 or 11, wherein one of W³ and W⁵ is N; the other W³ and W⁵ is independently selected from CH or C(halo); and each of W² and W⁶ is independently selected from CH and C(halo).

13. The compound according to any one of claims 1-12, wherein R⁴ is selected from halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, C₁-C₆ alkoxy, and —SO₂(R⁴⁵).

14. The compound according to any one of claims 1-13, wherein R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵).

15. The compound according to any one of claims 1-14, wherein R⁴ is —CO₂H.

16. The compound according to any one of claims 1-14, wherein R⁴ is —CO₂R⁴¹.

17. The compound of claim 16, wherein R⁴¹ is C₁-C₈ alkyl (e.g., R⁴¹ is CH₃).

18. The compound according to any one of claims 1-14, wherein R⁴ is —SO₂(R⁴⁵).

19. The compound of claim 18, wherein R⁴⁵ is C₁-C₈ alkyl.

20. The compound according to any one of claims 1-14, wherein R⁴ is —C(O)N(R⁴²)(R⁴³).

21. The compound of claim 20, wherein each of R⁴² and R⁴³ is independently selected from:
(i) hydrogen;
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected cycloalkyl, and heterocyclyl is optionally substituted with from 1-3 R^{c}.

22. The compound of claim 21, wherein one of R⁴² and R⁴³ is hydrogen; and the other of R⁴² and R⁴³ is C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 R^{c}.

23. The compound of claim 22, wherein one of R⁴² and R⁴³ is hydrogen; and the other of R⁴² and R⁴³ is C₁-C₈ alkyl, which is optionally substituted with from 1-3 R^{c}.

24. The compound of claim 23, wherein R^{c} at each occurrence is, independently, —OH; C₁-C₆ alkoxy (e.g., OCH₃); —C(O)(C₁-C₆ alkyl); or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein said heterocyclyl is optionally substituted with from 1-3 substituents independently selected from —OH and C₁-C₄ alkyl.

25. The compound of claim 22, wherein one of R⁴² and R⁴³ is hydrogen; and the other of R⁴² and R⁴³ is C₃-C₈ cycloalkyl; or heterocyclyl containing from 3-8 ring atoms, wherein from 1-2 of the ring atoms is independently selected from N, NH, N(C₁-C₆ alkyl), O, and S; and wherein each of said cycloalkyl or heterocyclyl is optionally substituted with from 1-3 R^{c}.

26. The compound of claim 20, wherein R⁴²—N—R⁴³ together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{c}.

27. The compound according to any one of claims 1-14, wherein R⁴ is heterocyclyloxy, each containing from 3-8 ring atoms, wherein from 1-2 of the ring atoms is independently selected from N, NH, N(C₁-C₆ alkyl), O, and S; and wherein said heterocyclyloxy is optionally substituted with from 1-3 independently selected R^{a}.

28. The compound according to any one of claims 1-27, wherein R⁵ and R⁶ are defined according to (C).

29. The compound of claim 28, wherein R⁵ and R⁶, together with the carbon atom to which each is attached, is C₃-C₈ cycloalkyl, which is optionally substituted with from 1-5 R^{c}.

30. The compound of claim 29, wherein R⁵ and R⁶, together with the carbon atom to which each is attached, is C₃-C₆ cycloalkyl, which is optionally substituted with from 1-5 R^{c}.

31. The compound of claim 29, wherein R⁵ and R⁶, together with the carbon atom to which each is attached, is C₆ cycloalkyl, which is optionally substituted with from 1-5 R^{c}.

32. The compound according to any one of claims 28-31, wherein R^{c} at each occurrence is, independently, —OH or C₁-C₆ alkyl.

33. The compound according to any one of claims 1-27, wherein R⁵ and R⁶ are defined according to (D).

34. The compound of claim 33, wherein R⁵ is C₃-C₈ cycloalkyl, which is optionally substituted with from 1-5 R^{c}.

35. The compound of claim 33 or 34, wherein W⁶ is C₁-C₆ alkyl, which is optionally substituted with a substituent selected from —OH and —CN.

36. The compound of claim 35, wherein R⁶ is —CH₂CH₃.

37. The compound of claim 35, wherein R⁶ is —CH₃.

38. The compound according to any one of claims 1-37, wherein the carbon attached to R⁵ and R⁶ has the S configuration.

39. The compound according to any one of claims 1-38, wherein R³ is C₆-C₁₀ aryl, which is optionally substituted with from 1-3 independently selected R^{d}.

40. The compound of claim 39, wherein R³ is phenyl that is substituted with 1 or 2 independently selected R^{d}.

41. The compound of claim 40, wherein R³ is 4-chloro-phenyl, 4-fluoro-phenyl, or 2,4-difluorophenyl.

42. The compound according to any one of claims 1-38, wherein R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}.

43. The compound of claim 42, wherein R³ is heteroaryl containing from 5-6 ring atoms, wherein from 1-4 of the ring atoms is independently selected from N, NH, N(C₁-C₆ alkyl), O, and S; and wherein said heteroaryl ring is substituted with 1 or 2 independently selected R^{d}.

44. The compound of claim 40, wherein R³ is thienyl, which is substituted with 1 or 2 independently selected R^{d}.

45. The compound according to any one of claims 39, 40, and 42-44, wherein R^{d} at each occurrence is independently selected from halo.

46. The compound according to any one of claims 1-45, wherein A is CH₂.

47. The compound according to any one of claims 1-45, wherein the compound is selected from the title compounds of Examples 1-90.

48. A pharmaceutical composition comprising a compound of formula (I), or a pharmaceutically acceptable salt thereof, as claimed in any one of claims 1-47, and a pharmaceutically acceptable carrier.

49. A method for treating a neurodegenerative disorder subject having, or at risk of having a neurodegenerative disorder, which comprises administering to the subject having, or at risk of having a neurodegenerative disorder a therapeutically effective amount of a compound of formula (I), or a pharmaceutically acceptable salt thereof, as claimed in any one of claims 1-47.

50. The compound, or a pharmaceutically acceptable salt thereof, as claimed in any one of claims 1-47 for use in the treatment of a neurodegenerative disorder.

51. Use of a compound, or a pharmaceutically acceptable salt thereof, of any of claims 1-47 in the manufacture of a medicament for the treatment of a neurodegenerative disorder.

52. The method of claim 49, the compound for use of claim 50, or the use of claim 51, wherein the neurodegenerative disorder is Alzheimer's disease.

53. The compound, or a pharmaceutically acceptable salt thereof, as claimed in any one of claims 1-47 for use in therapy.
